(19) United States
(12) Patent Application Publication
(10) Pub. No.: US 2025/0003940 A1
(43) Pub. Date: Jan. 2, 2025

(54) BIOSENSOR, BIOSENSOR COMPONENTS, AND USE THEREOF

(71) Applicant: VICTORIA LINK LIMITED, Wellington (NZ)

(72) Inventors: Wayne PATRICK, Wellington (NZ); Lee Ann Zandra TEJADA, Wellington (NZ)

(73) Assignee: VICTORIA LINK LIMITED, Wellington (NZ)

(21) Appl. No.: 18/708,767

(22) PCT Filed: Nov. 9, 2022
(86) PCT No.: PCT/NZ2022/050139

§ 371 Date: May 9, 2024

Related U.S. Application Data
(60) Provisional application No. 63/277,554, filed on Nov. 9, 2021.

(30) Foreign Application Priority Data
Nov. 10, 2021 (AU) 2021903582

(51) Int. Cl. G01N 33/14; C12N 9/00; C12N 9/78; C12Q 1/25; C12Q 1/34; G01N 33/18; G01N 33/68

(52) U.S. Cl. CPC G01N 33/143; C12N 9/78; C12N 9/93; C12Q 1/25; C12Q 1/34; G01N 33/188; G01N 33/245; G01N 33/6812; C12Y 305/03006; C12Y 603/01002

(57) ABSTRACT

Biosensor systems for detecting levels of nitrogen-containing molecules are provided. Further provided are biosensor system components and various detection platforms, methods, and kits.

### RELATED APPLICATIONS

This application claims the benefit of U.S. patent application No. 63/277,554 filed on 9 Nov. 2021, and Australian patent application number 2021903582 filed on 10 Nov. 2021, both of which are hereby incorporated by reference herein, in their entirety.

### FIELD

The present disclosure relates generally to biosensor systems for measuring levels of nitrogen-containing molecules in a sample. The disclosure further relates to the components of these systems, kits comprising these components, and the preparation and use of the same.

### BACKGROUND

Nitrogen is a key player in fermentation kinetics, and nitrogen levels affect the quality for various fermentation products, including wine. Specifically, to ensure high quality wine is produced, levels of nitrogen and other biomolecules in grapes (pre- and post-harvest) during the wine fermentation process must be monitored. These biomolecules serve as the substrates as well as by-products of fermentation. Aside from sugars, nitrogen-containing compounds are the most important nutrients that yeast require for fermentation (Hannam et al., 2013; Bell & Henschke, 2005).

Wine fermentation is the process of transforming grape must into wine by yeast. The most commonly used yeast for winemaking is Saccharomyces cerevisiae, and this organism requires a relatively high amount of nutrients such as sugars and nitrogen-containing compounds to support the process of fermentation. Typically, with enough nutrients, S. cerevisiae can produce about 12-15% v/v ethanol (Vilanova et al., 2007). Through the years, S. cerevisiae has been studied and domesticated to adapt to different kinds of stressful growth conditions to achieve a complete fermentation (Fleet, 2003). However, there are still instances of incomplete fermentation together with a high concentration of (>2-5 g/L) unfermented sugars as a result of stuck or sluggish fermentation (Querol, 2003). This occurs due to low nitrogen and nitrogen-containing compounds present in grape must before fermentation starts.

Yeast assimilable nitrogen (YAN) comprises nitrogenous compounds that can be metabolised by yeast during fermentation. YAN consists of ammonium, amino acids, and di- and tri-peptides. Amongst YAN constituents, ammonium and free amino acids (FAN), primarily: arginine and glutamine, are the most abundant sources of nitrogen (Nicolini et al. 2004; Crépin et al. 2012; Boudreau et al. 2017). According to Henschke et al. (1993), an optimal level of YAN is suggested to be 400-500 mg/L of grape must. Low YAN often affects the rate of fermentation and in severe cases leads to the production of hydrogen sulfide and ethyl carbamate by-products that affect taste and safety (Jackson, 2008). Thus, the addition of diammonium phosphate (DAP) is a normal practice for the winemakers to increase the total YAN concentration before fermentation begins (Bell & Henschke, 2005).

Currently, there are several methods seeking to measure YAN levels in wine grapes and must on the market (Bell & Henschke 2005). Detection consists of independent FAN and ammonium measurements. HPLC analysis of FAN is the most accurate; however it is not suitable for routine use due to its complicated and time-consuming process. Companies such as Megazyme and Unitech Scientific have developed enzymatic test kits for quantification of FAN and ammonium. These, however, require a UV/vis spectrophotometer and therefore are not suitable for onsite tests. Overall, the current suite of tests is slow, expensive, and highly complicated, and requires wine-makers to send their samples away to be tested in commercial laboratories. Many industries have similar issues and need improved means for nitrogen testing.

The present disclosure seeks to address these needs or at least to provide the public with a useful alternative.

### SUMMARY

As described herein, the present inventors have developed assays using two- and three-enzyme systems. A two-enzyme system includes BpsA (blue pigment synthetase A) and GS (glutamine synthetase). A three-enzyme system includes BpsA (blue pigment synthetase A), GS (glutamine synthetase), and ArgZ (arginine dihydrolase). These systems can be used, for example, to detect and measure key constituents of yeast assimilable nitrogen. As exemplifications, the disclosed systems are particularly useful for assessing and monitoring fermentation of alcoholic beverages, such as wines, and for assessing and monitoring the ripening/ripeness of grapes. Other types of testing are also provided for the systems of this disclosure.

The methods of this disclosure utilise (1) glutamine synthetase to convert glutamate and ammonium to glutamine, and (2) blue pigment synthetase A to convert the glutamine to the blue pigment indigoidine (two-enzyme system); or (1) arginine dihydrolase to convert arginine to ammonium, (2) glutamine synthetase to convert glutamate and ammonium to glutamine, and (3) blue pigment synthetase A to convert the glutamine to the blue pigment indigoidine (three-enzyme system). This combined enzymatic activity is then used to assess levels of key nitrogen-containing molecules, including key constituents of yeast assimilable nitrogen, in quantitative or qualitative assays.

In one particular aspect, the present disclosure encompasses a method of detecting levels of nitrogen-containing molecules in a sample, the nitrogen-containing molecules being selected from the group consisting of arginine, ammonium, glutamate, glutamine, and combinations thereof,
the method comprising incubating the sample with:
(i) a glutamine synthetase (GS) enzyme and a blue pigment synthetase A (BpsA) enzyme,
the incubating being under conditions whereby the GS enzyme is capable of capable of converting glutamate and ammonium into glutamine, and the BpsA enzyme is capable of converting glutamine to indigoidine pigment, or
(ii) an arginine dihydrolase (ArgZ) enzyme, a glutamine synthetase (GS) enzyme, and a blue pigment synthetase A (BpsA) enzyme,
the incubating being under conditions whereby the ArgZ enzyme is capable of converting arginine to ornithine and ammonium, the GS enzyme is capable of capable of converting glutamate and ammonium into glutamine, and the BpsA enzyme is capable of converting glutamine to indigoidine pigment, and
analysing the indigoidine pigment produced by quantitative or qualitative means to determine the amount of indigoidine pigment produced, thereby assessing:
for (i), levels of ammonium and glutamine in the sample, or levels of glutamate and glutamine in the sample,
for (ii) levels of arginine, ammonium, and glutamine in the sample, or levels of arginine, glutamate, and glutamine in the sample, and
thereby detecting the levels of the nitrogen-containing molecules in the sample.

In various aspects:

The method is utilised to detect the levels of the nitrogen-containing molecules (i) for yeast assimilable nitrogen testing, (ii) for water testing, (iii) for soil testing, or (iv) for biological sample testing.

The BpsA enzyme comprises an amino acid sequence of SEQ ID NO: 1, or an amino acid sequence sharing at least 80%, at least 90%, at least 95%, or at least 99% sequence identity to an amino acid sequence of SEQ ID NO: 1.

The BpsA enzyme of a preceding aspect, which includes an oxidation domain motif: (i) K-Y/F-G/R/T/Q/A-Y-A/P/G-S-A/P-G-G/A/S-L/I/T/S-Y-P/A/G-V/T-Q (SEQ ID NO: 21); and (ii) G-Y/F/H/A/T/S-Y/H-Y-Y/V-H/Q/D-P-V/F/L/K/A-E/R/H/Q-H/N-R/Q/T/E-L (SEQ ID NO: 22).

The BpsA enzyme consists of an amino acid sequence of SEQ ID NO: 1.

The GS enzyme comprises an amino acid sequence of any one of SEQ ID NO: 2 and SEQ ID NO: 13-14, or an amino acid sequence sharing at least 80%, at least 90%, at least 95%, or at least 99% sequence identity to an amino acid sequence of any one of SEQ ID NO: 2 and SEQ ID NO: 13-14.

The GS enzyme of a preceding aspect, which includes an adenylation site sequence motif N/D-L-F-D/E/K-L-P (SEQ ID NO: 19).

The GS enzyme consists of an amino acid sequence of any one of SEQ ID NO: 2 and SEQ ID NO: 13-14.

The ArgZ enzyme comprises an amino acid sequence of any one of SEQ ID NO: 3 and SEQ ID NO: 7-9, or an amino acid sequence sharing at least 80%, at least 90%, at least 95%, or at least 99% sequence identity to an amino acid sequence of any one of SEQ ID NO: 3 and SEQ ID NO: 7-9.

The ArgZ enzyme of a preceding aspect, which includes an arginine dihydrolase motif V-F-T/A-A-N-A/C (SEQ ID NO: 20).

The ArgZ enzyme consists of amino acid sequence of any one of SEQ ID NO: 3 and SEQ ID NO: 7-9

The method employs a concentration ratio of about 1:1 to about 1:100 for any of the BpsA enzyme, the GS enzyme, or the ArgZ enzyme.

The method is performed in one or more fluid receptacles.

The one or more fluid receptacles comprise tubes, wells, multi-well plates, or bottles.

The method is performed on one or more solid substrates.

The one or more solid substrates comprise silica, nitrocellulose, or paper.

The one or more solid substrates comprise strips, disks, beads, columns, chips, slides, arrays, or dipsticks.

The conditions comprise a pH range of about 6.5 to about 10.

The conditions comprise a pH of about 8.5.

The sample comprises fruit or components of fruit.

The sample comprises grapes, grape must, and/or grape juice.

The grapes are harvested grapes.

The grapes are pre-harvest grapes.

The sample comprises Pinot Gris, Pinot Noir, Sauvignon Blanc, Chardonnay, or other grapes, grape must, and/or grape juice.

The sample is a fermentation sample, a water sample, a soil sample, or a biological sample.

The fermentation sample is a sample from winemaking, beermaking, cidermaking, or spirits production.

The fermentation sample is a sample from a composition comprising fermenting fruit or grains.

The fermentation sample is a sample from a composition comprising fermenting grapes, grape must, and/or grape juice.

The fermentation sample is a sample from a composition comprising Pinot Gris, Pinot Noir, Sauvignon Blanc, Chardonnay or other grapes, grape must, and/or grape juice.

In one other aspect, the present disclosure encompasses a method of detecting levels of nitrogen-containing molecules in a sample,
the nitrogen-containing molecules being selected from the group consisting of arginine, ammonium, glutamate, glutamine, and combinations thereof,
the method comprising incubating the sample with:
(i) a glutamine synthetase (GS) enzyme and a blue pigment synthetase A (BpsA) enzyme,
the incubating being under conditions whereby the GS enzyme is capable of capable of converting glutamate and ammonium into glutamine, and the BpsA enzyme is capable of converting glutamine to indigoidine pigment, or
(ii) an arginine dihydrolase (ArgZ) enzyme, a glutamine synthetase (GS) enzyme, and a blue pigment synthetase A (BpsA) enzyme,
the incubating being under conditions whereby the ArgZ enzyme is capable of converting arginine to ornithine and ammonium, the GS enzyme is capable of capable of converting glutamate and ammonium into glutamine, and the BpsA enzyme is capable of converting glutamine to indigoidine pigment, and
quantifying the amount of indigoidine pigment produced under the conditions, thereby measuring:
for (i), levels of ammonium and glutamine in the sample, or levels of glutamate and glutamine in the sample,
for (ii) levels of arginine, ammonium, and glutamine in the sample, or levels of arginine, glutamate, and glutamine in the sample, and
thereby detecting the levels of the nitrogen-containing molecules in the sample.

In various aspects:

The method is utilised to detect the levels of the nitrogen-containing molecules (i) for yeast assimilable nitrogen testing, (ii) for water testing, (iii) for soil testing, or (iv) for biological sample testing.

The BpsA enzyme comprises an amino acid sequence of SEQ ID NO: 1, or an amino acid sequence sharing at least 80%, at least 90%, at least 95%, or at least 99% sequence identity to an amino acid sequence of SEQ ID NO: 1.

The BpsA enzyme of a preceding aspect, which includes an oxidation domain motif: (i) K-Y/F-G/R/T/Q/A-Y-A/P/G-S-A/P-G-G/A/S-L/I/T/S-Y-P/A/G-V/T-Q (SEQ ID NO: 21); and (ii) G-Y/F/H/A/T/S-Y/H-Y-Y/V-H/Q/D-P-V/F/L/K/A-E/R/H/Q-H/N-R/Q/T/E-L (SEQ ID NO: 22).

The BpsA enzyme consists of an amino acid sequence of SEQ ID NO: 1.

The GS enzyme comprises an amino acid sequence of any one of SEQ ID NO: 2 and SEQ ID NO: 13-14, or an amino acid sequence sharing at least 80%, at least 90%, at least 95%, or at least 99% sequence identity to an amino acid sequence of any one of SEQ ID NO: 2 and SEQ ID NO: 13-14.

The GS enzyme of a preceding aspect, which includes an adenylation site sequence motif N/D-L-F-D/E/K-L-P (SEQ ID NO: 19).

The GS enzyme consists of an amino acid sequence of any one of SEQ ID NO: 2 and SEQ ID NO: 13-14.

The ArgZ enzyme comprises an amino acid sequence of any one of SEQ ID NO: 3 and SEQ ID NO: 7-9, or an amino acid sequence sharing at least 80%, at least 90%, at least 95%, or at least 99% sequence identity to an amino acid sequence of any one of SEQ ID NO: 3 and SEQ ID NO: 7-9.

The ArgZ enzyme of a preceding aspect, which includes an arginine dihydrolase motif V-F-T/A-A-N-A/C (SEQ ID NO: 20).

The ArgZ enzyme consists of amino acid sequence of any one of SEQ ID NO: 3 and SEQ ID NO: 7-9

The method employs a concentration ratio of about 1:1 to about 1:100 for any of the BpsA enzyme, the GS enzyme, or the ArgZ enzyme.

The method is performed in one or more fluid receptacles.

The one or more fluid receptacles comprise tubes, wells, multi-well plates, or bottles.

The method is performed on one or more solid substrates.

The one or more solid substrates comprise silica, nitrocellulose, or paper.

The one or more solid substrates comprise strips, disks, beads, columns, chips, slides, arrays, or dipsticks.

The conditions comprise a pH range of about 6.5 to about 10.

The conditions comprise a pH of about 8.5.

The sample comprises fruit or components of fruit.

The sample comprises grapes, grape must, and/or grape juice.

The grapes are harvested grapes.

The grapes are pre-harvest grapes.

The sample comprises Pinot Gris, Pinot Noir, Sauvignon Blanc, Chardonnay or other grapes, grape must, and/or grape juice.

The sample is a fermentation sample, a water sample, a soil sample, or a biological sample.

The fermentation sample is a sample from winemaking, beermaking, cidermaking, or spirits production.

The fermentation sample is a sample from a composition comprising fermenting fruit or grains.

The fermentation sample is a sample from a composition comprising fermenting grapes, grape must, and/or grape juice.

The fermentation sample is a sample from a composition comprising Pinot Gris, Pinot Noir, Sauvignon Blanc, Chardonnay or other grapes, grape must, and/or grape juice.

In yet one other aspect, the present disclosure encompasses a biosensor system for detecting levels of nitrogen-containing molecules in a sample,
the nitrogen-containing molecules being selected from the group consisting of arginine, ammonium, glutamate, and glutamine,
the system comprising:
(i) a glutamine synthetase (GS) enzyme and a blue pigment synthetase A (BpsA) enzyme, or
(ii) an arginine dihydrolase (ArgZ) enzyme, a glutamine synthetase (GS) enzyme, and a blue pigment synthetase A (BpsA) enzyme.

In various aspects:

The biosensor system provides for detecting the levels of the nitrogen-containing molecules (i) for yeast assimilable nitrogen testing, (ii) for water testing, (iii) for soil testing, or (iv) for biological sample testing.

The BpsA enzyme comprises an amino acid sequence of SEQ ID NO: 1, or an amino acid sequence sharing at least 80%, at least 90%, at least 95%, or at least 99% sequence identity to an amino acid sequence of SEQ ID NO: 1.

The BpsA enzyme of a preceding aspect, which includes an oxidation domain motif: (i) K-Y/F-G/R/T/Q/A-Y-A/P/G-S-A/P-G-G/A/S-L/I/T/S-Y-P/A/G-V/T-Q (SEQ ID NO: 21); and (ii) G-Y/F/H/A/T/S-Y/H-Y-Y/V-H/Q/D-P-V/F/L/K/A-E/R/H/Q-H/N-R/Q/T/E-L (SEQ ID NO: 22).

The BpsA enzyme consists of an amino acid sequence of SEQ ID NO: 1.

The GS enzyme comprises an amino acid sequence of any one of SEQ ID NO: 2 and SEQ ID NO: 13-14, or an amino acid sequence sharing at least 80%, at least 90%, at least 95%, or at least 99% sequence identity to an amino acid sequence of any one of SEQ ID NO: 2 and SEQ ID NO: 13-14.

The GS enzyme of a preceding aspect, which includes an adenylation site sequence motif N/D-L-F-D/E/K-L-P (SEQ ID NO: 19).

The GS enzyme consists of an amino acid sequence of any one of SEQ ID NO: 2 and SEQ ID NO: 13-14.

The ArgZ enzyme comprises an amino acid sequence of any one of SEQ ID NO: 3 and SEQ ID NO: 7-9, or an amino acid sequence sharing at least 80%, at least 90%, at least 95%, or at least 99% sequence identity to an amino acid sequence of any one of SEQ ID NO: 3 and SEQ ID NO: 7-9.

The ArgZ enzyme of a preceding aspect, which includes an arginine dihydrolase motif V-F-T/A-A-N-A/C (SEQ ID NO: 20).

The ArgZ enzyme consists of amino acid sequence of any one of SEQ ID NO: 3 and SEQ ID NO: 7-9

The biosensor system utilises a concentration ratio of about 1:1 to about 1:100 for any of the BpsA enzyme, the GS enzyme, or the ArgZ enzyme.

The ArgZ enzyme, the GS enzyme, the BpsA enzyme, or any combination of these is provided in one or more fluid receptacles.

The one or more fluid receptacles comprise tubes, wells, multi-well plates, or bottles.

The ArgZ enzyme, the GS enzyme, the BpsA enzyme, or any combination of these is provided on one or more solid substrates.

The one or more solid substrates comprise silica, nitrocellulose, or paper.

The one or more solid substrates comprise strips, disks, beads, columns, chips, slides, arrays, or dipsticks.

The ArgZ enzyme, the GS enzyme, the BpsA enzyme, or any combination of these is provided in a composition.

The composition is a tablet, a powder, or a solution.

The composition includes one or more binders, buffers, co-factors, diluents, salts, stabilisers, and/or excipients.

Each enzyme is combined simultaneously in the biosensor system.

Each enzyme is combined sequentially in the biosensor system.

Each enzyme is combined in any order and in any sequence in the biosensor system.

The sample comprises fruit or components of fruit.

The sample comprises grapes, grape must, and/or grape juice.

The grapes are harvested grapes.

The grapes are pre-harvest grapes.

The sample comprises Pinot Gris, Pinot Noir, Sauvignon Blanc, Chardonnay, or other grapes, grape must, and/or grape juice.

The sample is a fermentation sample, a water sample, a soil sample, or a biological sample.

The fermentation sample is a sample from winemaking, beermaking, cidermaking, or spirits production.

The fermentation sample is a sample from a composition comprising fermenting fruit or grains.

The fermentation sample is a sample from a composition comprising fermenting grapes, grape must, and/or grape juice.

The fermentation sample is a sample from a composition comprising Pinot Gris, Pinot Noir, Sauvignon Blanc, or Chardonnay grapes, grape must, and/or grape juice.

In yet one other aspect, the present disclosure encompasses a kit for detecting levels nitrogen-containing molecules in a sample,
the nitrogen-containing molecules being selected from the group consisting of arginine, ammonium, glutamate, glutamine, and combinations thereof,
the kit comprising:
(i) a glutamine synthetase (GS) enzyme and a blue pigment synthetase A (BpsA) enzyme, or
(ii) an arginine dihydrolase (ArgZ) enzyme, a glutamine synthetase (GS) enzyme, and a blue pigment synthetase A (BpsA) enzyme.

In various aspects:

The kit provides for detecting the levels of the nitrogen-containing molecules (i) for yeast assimilable nitrogen testing, (ii) for water testing, (iii) for soil testing, or (iv) for biological sample testing.

The BpsA enzyme comprises an amino acid sequence of SEQ ID NO: 1, or an amino acid sequence sharing at least 80%, at least 90%, at least 95%, or at least 99% sequence identity to an amino acid sequence of SEQ ID NO: 1.

The BpsA enzyme of a preceding aspect, which includes an oxidation domain motif: (i) K-Y/F-G/R/T/Q/A-Y-A/P/G-S-A/P-G-G/A/S-L/I/T/S-Y-P/A/G-V/T-Q (SEQ ID NO: 21); and (ii) G-Y/F/H/A/T/S-Y/H-Y-Y/V-H/Q/D-P-V/F/L/K/A-E/R/H/Q-H/N-R/Q/T/E-L (SEQ ID NO: 22).

The BpsA enzyme consists of an amino acid sequence of SEQ ID NO: 1.

The GS enzyme comprises an amino acid sequence of any one of SEQ ID NO: 2 and SEQ ID NO: 13-14, or an amino acid sequence sharing at least 80%, at least 90%, at least 95%, or at least 99% sequence identity to an amino acid sequence of any one of SEQ ID NO: 2 and SEQ ID NO: 13-14.

The GS enzyme of a preceding aspect, which includes an adenylation site sequence motif N/D-L-F-D/E/K-L-P (SEQ ID NO: 19).

The GS enzyme consists of an amino acid sequence of any one of SEQ ID NO: 2 and SEQ ID NO: 13-14.

The ArgZ enzyme comprises an amino acid sequence of any one of SEQ ID NO: 3 and SEQ ID NO: 7-9, or an amino acid sequence sharing at least 80%, at least 90%, at least 95%, or at least 99% sequence identity to an amino acid sequence of any one of SEQ ID NO: 3 and SEQ ID NO: 7-9.

The ArgZ enzyme of a preceding aspect, which includes an arginine dihydrolase motif V-F-T/A-A-N-A/C (SEQ ID NO: 20).

The ArgZ enzyme consists of amino acid sequence of any one of SEQ ID NO: 3 and SEQ ID NO: 7-9

The kit provides a concentration ratio of about 1:1 to about 1:100 for any of the BpsA enzyme, the GS enzyme, or the ArgZ enzyme.

The ArgZ enzyme, the GS enzyme, the BpsA enzyme, or any combination of these is provided in one or more fluid receptacles.

The one or more fluid receptacles comprise tubes, wells, multi-well plates, or bottles.

The ArgZ enzyme, the GS enzyme, the BpsA enzyme, or any combination of these is provided on one or more solid substrates.

The one or more solid substrates comprise silica, nitrocellulose, or paper.

The one or more solid substrates comprise strips, disks, beads, columns, chips, slides, arrays, or dipsticks.

The ArgZ enzyme, the GS enzyme, the BpsA enzyme, or any combination of these is provided in a composition.

The composition is a tablet, a powder, or a solution.

The composition includes one or more binders, buffers, co-factors, diluents, salts, stabilisers, and/or excipients.

The ArgZ enzyme, the GS enzyme, the BpsA enzyme, or any combination of these is provided in a freeze-dried form.

The freeze-dried form includes mannitol.

The ArgZ enzyme, the GS enzyme, and the BpsA enzyme, provided as one or more combinations in the kit.

The ArgZ enzyme, the GS enzyme, and the BpsA enzyme are provided separately in the kit.

The sample comprises fruit or components of fruit.

The sample comprises grapes, grape must, and/or grape juice.

The grapes are harvested grapes.

The grapes are pre-harvest grapes.

The sample comprises Pinot Gris, Pinot Noir, Sauvignon Blanc, Chardonnay or other grapes, grape must, and/or grape juice.

The sample is a fermentation sample, a water sample, a soil sample, or a biological sample.

The fermentation sample is a sample from winemaking, beermaking, cidermaking, or spirits production.

The fermentation sample is a sample from a composition comprising fermenting fruit or grains.

The fermentation sample is a sample from a composition comprising fermenting grapes, grape must, and/or grape juice.

The fermentation sample is a sample from a composition comprising Pinot Gris, Pinot Noir, Sauvignon Blanc, Chardonnay or other grapes, grape must, and/or grape juice.

The kit comprises one or more of: buffers, ATP, MgCl₂, and KCl, containers, and instructions for use.

In yet one other aspect, the present disclosure encompasses a combination of components for detecting levels nitrogen-containing molecules in a sample,
the nitrogen-containing molecules being selected from the group consisting of arginine, ammonium, glutamate, glutamine, and combinations thereof,
the combination comprising:
(i) a glutamine synthetase (GS) enzyme and a blue pigment synthetase A (BpsA) enzyme, or
(ii) an arginine dihydrolase (ArgZ) enzyme, a glutamine synthetase (GS) enzyme, and a blue pigment synthetase A (BpsA) enzyme.

In various aspects:

The combination provides for detecting the levels of the nitrogen-containing molecules (i) for yeast assimilable nitrogen testing, (ii) for water testing, (iii) for soil testing, or (iv) for biological sample testing.

The BpsA enzyme comprises an amino acid sequence of SEQ ID NO: 1, or an amino acid sequence sharing at least 80%, at least 90%, at least 95%, or at least 99% sequence identity to an amino acid sequence of SEQ ID NO: 1.

The BpsA enzyme of a preceding aspect, which includes an oxidation domain motif: (i) K-Y/F-G/R/T/Q/A-Y-A/P/G-S-A/P-G-G/A/S-L/I/T/S-Y-P/A/G-V/T-Q (SEQ ID NO: 21); and (ii) G-Y/F/H/A/T/S-Y/H-Y-Y/V-H/Q/D-P-V/F/L/K/A-E/R/H/Q-H/N-R/Q/T/E-L (SEQ ID NO: 22).

The BpsA enzyme consists of an amino acid sequence of SEQ ID NO: 1.

The GS enzyme comprises an amino acid sequence of any one of SEQ ID NO: 2 and SEQ ID NO: 13-14, or an amino acid sequence sharing at least 80%, at least 90%, at least 95%, or at least 99% sequence identity to an amino acid sequence of any one of SEQ ID NO: 2 and SEQ ID NO: 13-14.

The GS enzyme of a preceding aspect, which includes an adenylation site sequence motif N/D-L-F-D/E/K-L-P (SEQ ID NO: 19).

The GS enzyme consists of an amino acid sequence of any one of SEQ ID NO: 2 and SEQ ID NO: 13-14.

The ArgZ enzyme comprises an amino acid sequence of any one of SEQ ID NO: 3 and SEQ ID NO: 7-9, or an amino acid sequence sharing at least 80%, at least 90%, at least 95%, or at least 99% sequence identity to an amino acid sequence of any one of SEQ ID NO: 3 and SEQ ID NO: 7-9.

The ArgZ enzyme of a preceding aspect, which includes an arginine dihydrolase motif V-F-T/A-A-N-A/C (SEQ ID NO: 20).

The ArgZ enzyme consists of amino acid sequence of any one of SEQ ID NO: 3 and SEQ ID NO: 7-9

The combination provides a concentration ratio of about 1:1 to about 1:100 for any of the BpsA enzyme, the GS enzyme, or the ArgZ enzyme.

At least one of the ArgZ enzyme, the GS enzyme, and the BpsA enzyme is provided in one or more fluid receptacles.

The one or more fluid receptacles comprise tubes, wells, multi-well plates, or bottles.

At least one of the ArgZ enzyme, the GS enzyme, and the BpsA enzyme is provided on one or more solid substrates.

The one or more solid substrates comprise silica, nitrocellulose, or paper.

The one or more solid substrates comprise strips, disks, beads, columns, chips, slides, arrays, or dipsticks.

At least one of the ArgZ enzyme, the GS enzyme, and the BpsA enzyme is provided in a composition.

The composition is a tablet, a powder, or a solution.

The composition includes one or more binders, buffers, co-factors, diluents, salts, stabilisers, and/or excipients.

At least one of the ArgZ enzyme, the GS enzyme, and the BpsA enzyme is provided in a freeze-dried form.

The freeze-dried form includes mannitol.

The sample comprises fruit or components of fruit.

The sample comprises grapes, grape must, and/or grape juice

The grapes are harvested grapes.

The grapes are pre-harvest grapes.

The sample comprises Pinot Gris, Pinot Noir, Sauvignon Blanc, Chardonnay or other grapes, grape must, and/or grape juice.

The sample is a fermentation sample, a water sample, a soil sample, or a biological sample.

The fermentation sample is a sample from winemaking, beermaking, cidermaking, or spirits production.

The fermentation sample is a sample from a composition comprising fermenting fruit or grains.

The fermentation sample is a sample from a composition comprising fermenting grapes, grape must, and/or grape juice.

The fermentation sample is a sample from a composition comprising Pinot Gris, Pinot Noir, Sauvignon Blanc, Chardonnay or other grapes, grape must, and/or grape juice.

The combination is prepared by providing each enzyme simultaneously.

The combination is prepared by providing each enzyme sequentially.

The combination is prepared by providing each enzyme in any combination and in any order.

The combination of components further comprises one or more of: buffers, ATP, MgCl₂, and KCl.

Novel features that are believed to be characteristic will be better understood from the detailed description when considered in connection with any accompanying figures and examples. However, the figures and examples provided herein are intended to help illustrate or assist with developing an understanding; these are not intended to limit the scope of this disclosure.

### BRIEF DESCRIPTION OF THE DRAWINGS

FIG. 1: Schematic diagram summarising the principal sources and sinks for nitrogen from vineyard to the finished wine, and the principal components of wine that are affected by nitrogen. (N, nitrogen; MLF, malolactic fermentation) (Bell & Henschke, 2005).

FIG. 2A: Schematic diagram of typical biosensor and components (Mehrotra, 2016).

FIG. 2B: Schematic diagram of biosensor system in one embodiment of the present disclosure.

FIG. 2C: Exemplary assay formats for the biosensor system of the present disclosure. Left panel shows microplate format for detecting glutamine, plus glutamate or ammonium in grapes (e.g., grape must or grape juice). This may be employed as a quantitative assay when using a spectrophotometer or similar measurement equipment. Right panels show filter paper disc format for detecting glutamine, plus glutamate or ammonium in grapes (e.g., grape must or grape juice). This may be utilised in semi-quantitative assays.

FIG. 3: GS activity assay. The assay was adapted from Shapiro et. al. The reaction mixture included 30 mM imidazole, 100 mM Na-glutamate, 8.5 mM adenosine 5′-triphosphate (ATP), 1 mM phosphoenolpyruvate (PEP), 60 mM magnesium chloride (MgCl₂), 20 mM potassium chloride (KCl), 45 mM ammonium chloride (NH₄Cl), 0.25 mM (3-nicotinamide adenine dinucleotide (NADH), 28 units pyruvate kinase/40 units L-lactic dehydrogenase (PK/LDH). The reactions were performed at 37° C. and rates were measured by the decrease of absorbance at 340 nm.

FIG. 4A: ATP concentration trial. The buffer used in the assay was 100 mM Tris pH 11.4. An excess of MgCl₂, KCl, Na-Glu, NH₄Cl, was in the reaction mix. GS concentration was 3 μM while BpsA concentration was 6 μM. The assay was carried out at 37° C. The rate was measured by the increase in the absorbance at 590 nm for 9 minutes. Data are the mean values of three replicates and error bars indicate the standard error of the mean.

FIG. 4B: MgCl₂ concentration trial. The reaction was in 100 mM Tris pH 11.4 with 15 mM ATP, and an excess amount of KCl, NH₄Cl, and Na-glu. GS concentration was 3 μM while BpsA concentration was 6 μM. The assay was carried out at 37° C. The rate was measured by the increase in the absorbance at 590 nm for 9 minutes. Data are the mean values of three replicates and error bars indicate the standard error of the mean.

FIG. 5: KCl concentration trial. The reaction consists of 100 mM Tris Base pH 11.4, 15 mM ATP, 20 mM MgCl₂, and an excess amount of NH₄Cl and Na-Glu. GS concentration was 3 μM while BpsA concentration was 6 μM in the assay. The assay was carried out at 37° C. The rate was measured by the increase in the absorbance at 590 nm for 9 minutes. Data are the mean values of three replicates and error bars indicate the standard error of the mean.

FIG. 6A: Sigmoidal kinetics data obtained when the GS from Helicobacter pylori (HpGS) is assayed in the presence of various concentrations of its substrate, glutamate. The concentration of GS and BpsA is 3 μM and 6 μM respectively. An optimised condition for the GS-BpsA assay was used. A range of glutamate concentrations (0-900 μM) was used in the assay. Rates were measured through the increase in absorbance at 590 nm at 37° C. Data are the mean values of three replicates and error bars indicate the standard error of the mean.

FIG. 6B: Sigmoidal kinetics data obtained when HpGS is assayed in the presence of various concentrations of its substrate, ammonium. The concentration of GS and BpsA is 3 μM and 6 μM respectively. An optimised condition for the GS-BpsA assay was used. A range of 0-900 μM NH₄Cl was used in the assay. Rates were measured through the increase in absorbance at 590 nm at 37° C. Data are the mean values of three replicates and error bars indicate the standard error of the mean.

FIG. 7: GS-BpsA tests with grape juice samples. Rows A and B correspond to the GS-BpsA test (detecting ammonium+glutamate+glutamine), while row C is for the BpsA test (detecting glutamine only). 100% grape juice sample tested. 1A-B is the pressed Pinot Gris, 2A-B is the white grape juice filtered, 3A-B is the white grape juice pre-treated with charcoal, and 4A-B is the red grape juice charcoaled.

FIG. 7B: GS-BpsA tests with grape juice samples. Rows A and B correspond to the GS-BpsA test (detecting ammonium+glutamate+glutamine), while row C is for the BpsA test (detecting glutamine only). Undiluted grape juice sample with 500 μM additional glutamate tested. 1A-B is the pressed pinot gris, 2A-B is the white grape juice filtered, 3A-B is the white grape juice charcoaled, and 4A-B is the red grape juice charcoaled.

FIG. 7C: GS-BpsA tests with grape juice samples. Rows A and B correspond to the GS-BpsA test (detecting ammonium+glutamate+glutamine), while row C is for the BpsA test (detecting glutamine only). Two-fold diluted grape juice sample was tested. 1A-B is the pressed pinot gris, 2A-B is the white grape juice filtered, 3A-B is the white grape juice charcoaled, and 4A-B is the red grape juice charcoaled.

FIG. 7D: GS-BpsA tests with grape samples. Rows A and B correspond to the GS-BpsA test (detecting ammonium+glutamate+glutamine), while row C is for the BpsA test (detecting glutamine only). Two-fold diluted grape juice sample was tested with 500 μM additional glutamate. 1A-B is the pressed pinot gris, 2A-B is the white grape juice filtered, 3A-B is the white grape juice charcoaled, and 4A-B is the red grape juice charcoaled.

FIG. 8A: GS-BpsA with wine grape samples. “PN” is Pinot Noir variety and “PG” is Pinot Gris. Different concentrations of wine grape samples (grape juices) were used. Ammonium and glutamate concentration can be inferred from the difference of indigoidine produced with the sample only and sample supplemented with excess sodium glutamate (i.e. +Na-Glu). The Na-Glu control was included to ensure that the added Na-Glu in the samples is pure and no glutamine contaminant was added.

FIG. 8B: BpsA with wine grape samples. This test can be used to illustrate the amount of glutamine present in the wine grape samples. The very pale blue colour observed in this particular assay corresponded with low glutamine concentrations in the samples that were tested.

FIG. 9A: Further optimisation of GS-BpsA test for wine grape samples. ATP optimisation. A varying concentration of ATP from 5 mM to 15 mM in the reaction was tested. The sample used was Pinot Gris that had been blended and filtered through muslin cloth. Ten millimolar glutamate was added for the complete conversion of all ammonium in the sample. The reaction was incubated for 1 hour before the resolubilisation of indigoidine with DMSO. Data are the mean values of three replicates and error bars indicate the standard error of the mean.

FIG. 9B: Further optimisation of GS-BpsA test for wine grape samples. Sample volume optimisation. Sample volumes of 1 μL, 5 μL, and 10 μL were tested in the assay. The reaction mixture was added until 40 μL with the same final concentration of each of the components. The sample used was Pinot Gris that had been blended and muslin-filtered. Ten millimolar glutamate was added for the complete conversion of all ammonium in the sample. The reaction was incubated for 1 hour before the resolubilisation of indigoidine with DMSO. Data are the mean values of three replicates and error bars indicate the standard error of the mean.

FIG. 10A: Incubation time optimisation and GS-BpsA concentration optimisation. A reaction mix with different GS-BpsA ratio was prepared and used for the test. The sample used for this test was Pinot Gris that had been blended and muslin-filtered. Ten millimolar glutamate was added for the complete conversion of ammonium. Aliquots of each reaction were taken at 20 minute intervals and indigoidine was resolubilised using DMSO, before measuring its absorbance at 590 nm. Data are the mean values of three replicates and error bars indicate the standard error of the mean.

FIG. 10B: An optimised GS-BpsA ratio and incubation time. Graph derived from FIG. 10A.

FIG. 11A: Quantification of ammonium in wine grape samples throughout fermentation. Ammonium quantification on Pinot Gris. The test was done side by side using the disclosed biosensor system with GS-BpsA, and using available kits from Unitech, and Megazyme, following each protocol.

FIG. 11B: Quantification of ammonium in wine grape samples throughout fermentation. Ammonium quantification on Pinot Noir. The test was done side by side using the disclosed biosensor system with GS-BpsA, and using available kits from Unitech, and Megazyme, following each protocol.

FIG. 12: Schematic for arginine deiminase enzymatic reaction.

FIG. 13: Schematic for arginine dihydrolase enzymatic reaction.

FIGS. 14A-B: Expression trial of arginine deiminase candidates. SDS-PAGE gels showing arginine deiminase polypeptides. Expression trials of A. MhADI. B. PpADI. The abbreviations corresponds to the following; PL—protein ladder, TP—Total protein, SP—soluble protein.

FIG. 15: ArgZ candidates' activity with GS and BpsA. Optimised GS/BpsA reaction conditions were used for the assay. Each ArgZ was present at 10 μM. The only source of nitrogen for indigoidine production was arginine (0.6 mM). Glutamate was present at 2 mM, so that the ammonium liberated from arginine could be incorporated into glutamine, and then indigoidine, by GS and BpsA respectively. Abbreviations: Ss, Synechocystis sp. PCC6803; Sc, S. coelicolor; Mt, M. trichosporium; and Rc, R. centenum. The two rows show results from technical duplicate assays.

FIG. 16A: Range and activity test for R. centenum ArgZ. R. centenum ArgZ coupled with GS and BpsA. An optimised GS/BpsA reaction condition was used for the assay with increasing concentrations of arginine. An excess of glutamate was added to ensure the complete conversion of the ammonium produced from arginine, into indigoidine.

FIG. 16B: Range and activity test for S. coelicolor ArgZ. S. coelicolor ArgZ coupled with GS and BpsA. An optimised GS/BpsA reaction condition was used for the assay with increasing concentrations of arginine. An excess of glutamate was added to ensure the complete conversion of the ammonium produced from arginine, into indigoidine.

FIG. 17: Michaelis-Menten kinetics of RcArgZ with arginine. A range of 0-3000 μM arginine concentration was used in the assay. Rates were measured through the decrease in absorbance at 340 nm at 37° C. Data are the mean values of three replicates and error bars indicate the standard error of the mean.

FIG. 18A: RcArgZ activity in wine grape samples. RcArgZ (detecting arginine) was coupled with GS and BpsA (detecting ammonium and glutamine) in the assay. The wine grape samples used were Pinot Noir (PN) and Pinot Gris (PG). The two rows are replicates.

FIG. 18B: RcArgZ activity in wine grape samples. GS/BpsA test (detecting ammonium and glutamine). The wine grape samples (grape juice) used were Pinot Noir (PN) and Pinot Gris (PG). The two rows are replicates.

FIGS. 19A-19B: Three-enzyme YAN biosensor versus commercial test kits for primary amino nitrogen (PAN). FIG. 19A shows the results from a Pinot Noir grape sample (grape juice), analysed with the two commercial kits and the disclosed biosensor system, respectively. FIG. 19B shows the results from a Pinot Gris sample, analysed with the same two kits and the disclosed biosensor system.

FIG. 20A: Amino acid sequence for Streptomyces lavendulae BpsA enzyme. SEQ ID NO: 1. The His-tag sequence used for the protein fusion is also shown.

FIG. 20B: Nucleotide sequence for Streptomyces lavendulae BpsA enzyme. SEQ ID NO: 4. The His-tag sequence used for the protein fusion is also shown.

FIG. 21A: Amino acid sequence for Helicobacter pylori glutamine synthetase enzyme. SEQ ID NO: 2. The His-tag sequence used for the protein fusion is also shown.

FIG. 21B: Nucleotide sequence for Helicobacter pylori glutamine synthetase enzyme. SEQ ID NO: 5. The His-tag sequence used for the protein fusion is also shown.

FIG. 22A: Amino acid sequence for Rhodospirillum centenum arginine dihydrolase enzyme. SEQ ID NO: 3. This sequence is derived from the sequence listed as accession number B6IRA8 in the UniProt database, but with the deletion of amino acid residues 2 to 18 from the UniProt sequence. These residues were deleted because they were predicted to encode a disordered region in the polypeptide. The His-tag sequence used for the protein fusion is also shown.

FIG. 22B: Nucleotide sequence encoding the Rhodospirillum centenum arginine dihydrolase enzyme. SEQ ID NO: 6. The nucleotide sequence encoding the His-tag used for the protein fusion is also shown.

FIG. 23A: RcArgZ activity levels after freeze-drying with different additives. RcArgZ was purified and exchanged into the freeze-drying buffers. The buffers contained 50 mM Tris-Cl pH 8.0, 1 mM MgCl₂, and one of the stabilisers. The stabilisers were the following: 1 mM mannitol, 40 mg/mL mannitol, 1 mM sucrose, 10 mg/mL sucrose, 1 mM trehalose, 20 mg/mL trehalose, or 20 mg/mL sucrose and 40 mg/mL mannitol. The sample was then snap frozen using liquid nitrogen before putting in a benchtop freeze-dryer. The sample was left drying for 18-24 hours. The activity of the freeze-dried enzymes was tested and compared with the non-freeze-dried standard (in their corresponding storage buffer).

FIG. 23B: HpGS activity levels after freeze-drying with different additives. HpGS was purified and buffer exchanged to the freeze-drying buffers. The buffers contained 50 mM Tris-Cl pH 8.0, 1 mM MgCl₂, and the stabilisers. The stabilisers were the following: 1 mM mannitol, 40 mg/mL mannitol, 1 mM sucrose, 10 mg/mL sucrose, 1 mM trehalose, 20 mg/mL trehalose, or 20 mg/mL sucrose and 40 mg/mL mannitol. The sample was then snap frozen using liquid nitrogen before putting in a benchtop freeze-dryer. The sample was left drying for 18-24 hours. The activity of the freeze-dried enzymes was tested and compared with the non-freeze-dried standard (in their corresponding storage buffer).

FIG. 23C: BpsA activity levels after freeze-drying with different additives. BpsA was purified and buffer exchanged to the freeze-drying buffers. The buffers contained 50 mM Tris-Cl pH 8.0, 1 mM MgCl₂, and the stabilisers. The stabilisers were the following: 1 mM mannitol, 40 mg/mL mannitol, 1 mM sucrose, 10 mg/mL sucrose, 1 mM trehalose, 20 mg/mL trehalose, or 20 mg/mL sucrose and 40 mg/mL mannitol. The sample was then snap frozen using liquid nitrogen before putting in a benchtop freeze-dryer. The sample was left drying for 18-24 hours. The activity of the freeze-dried enzymes was tested and compared with the non-freeze-dried standard (in their corresponding storage buffer).

FIG. 24: pH optima. One hundred millimolar phosphate, Tris-Cl, or CHES buffers were used to buffer assays at different pH levels. Each reaction included buffer, 5 mM NADH, 10 mM arginine, 10 mM α-ketoglutarate, and 70 U of glutamate dehydrogenase. The assay was started upon the addition of 1 μM RcArgZ at 25° C. The rate was measured by the decrease in absorbance at 340 nm for 5 minutes. Data are the mean values of three replicates and error bars indicate the standard error of the mean.

FIG. 25: Amino acid and nucleotide sequence information for ArgZ enzyme candidates.

FIG. 26A: Spectrophotometric assay for determining the limit of detection for nitrogen (in the form of NH₄Cl) using the three-enzyme biosensor. Data are plotted as the mean±SEM for n=3 replicates. Linear regression gave an R² value of 0.97, as shown on the plot.

FIG. 26B: Focused view of data from FIG. 26A, showing detection results for 0 to 1.25 mg/L nitrogen (in the form of NH₄Cl). Linear regression gave an R² value of 0.904, as shown on the plot.

FIG. 27: Spectrophotometric assays utilising different ratios and concentrations of HpGS and BpsA. Symbols in the graph are defined as follows: circle corresponds to Test 1; square corresponds to Test 2; triangle corresponds to Test 3; and inverted triangle corresponds to Test 4. See Table 6. Reaction mixtures contained 100 mM Tris base pH 11.4, 8 mM ATP, 10 mM KCl, 20 mM MgCl₂, and 10 mM glutamate, plus an aliquot (5 μL) of Pinot Gris grape juice as the source of ammonium. Data points for triplicate experiments are plotted, with the black lines corresponding to the mean values.

FIG. 28: Spectrophotometric assays utilising different ratios of RcArgZ, HpGS and BpsA as defined in Table 7. Each assay mixture contained 100 mM Tris pH 11.4, 20 mM MgCl₂, 10 mM KCl, 15 mM ATP and 1 mM glutamate. Each assay was started by adding 1 mM arginine and incubated for 1 hour at 25° C. DMSO (80% v/v) was added to stop the reaction and resolubilise the indigodine, before the amount of indigoidine was measured by recording the absorbance at 590 nm. The mean and SEM for triplicate experiments are plotted.

FIGS. 29A-29C: Comparisons of enzymes that were freshly prepared (left bar in each panel, darker shading) with enzymes that had been freeze-dried and then reconstituted (right bar in each panel, lighter shading). BpsA activity (FIG. 29A) was measured in an assay comprising 1 μM enzyme, 15 mM ATP, 20 mM MgCl₂, 10 mM KCl, and 1 mM glutamine. The assay was incubated for 5 minutes at 37° C. before DMSO was added to 80% (v/v) and the absorbance at 590 nm (A₅₉₀) was measured. HpGS and RcArgZ activities (FIGS. 29B and 29C, respectively) were both assayed under identical conditions, except as follows. For testing HpGS, it was present at 1 μM, with 5 μM BpsA as a coupled enzyme for colour development; the substrates (NH₄Cl and glutamate) were both present at 1 mM. For testing RcArgZ, it was present at 1 μM, with the coupled enzymes HpGS and BpsA each present at 5 μM; the substrate arginine and the coupling substrate glutamate were both present at 1 mM. Data are the mean values of three replicates. Error bars indicate the standard error of the mean.

FIGS. 30A-30B: Spectrophotometric assay for demonstrating the time dependence of indigoidine production by the three-enzyme biosensor. The assay mixture comprised 3 μM RcArgZ, 6 μM HpGS, 9 μM BpsA, 8 mM ATP, 20 mM MgCl₂, 10 mM KCl, 10 mM glutamate, and arginine corresponding to 150 mg/L nitrogen. Reaction mixtures were incubated at different timepoints (15-90 minutes) at 22° C. before DMSO was added to 80% (v/v). Absorbance measurements were normalised using a blank that lacked arginine and the data are the mean values of three replicates. Error bars indicate the standard error of the mean. FIG. 30A shows data from the entire experiment. FIG. 30B shows plots for the same data, but only from t=0 to t=60 minutes. Linear regression of these data gave an R² value of 0.99, as shown on the plot.

FIGS. 31A-31B: Testing of four GS enzymes (CgGS, EcGS, HpGS and TcGS) coupled with BpsA in a two-enzyme biosensor. FIG. 31A shows visual analysis of the indigoidine produced in the GS-BpsA coupled assay. Each column contains the enzymes as indicated; n=3 technical replicates. FIG. 31B shows the corresponding spectrophotometric quantification of the indigoidine produced in the GS-BpsA coupled assays. The reaction consisted of 0.1 M Tris pH 8.5, 20 mM MgCl₂, 1 mM glutamate, and 1 mM NH₄Cl. The GS concentration was 1 μM, and the BpsA concentration was 3 μM. The reaction was incubated for 30 minutes at 25° C. before indigoidine was resolubilised with DMSO. Symbols in the graph are defined as follows: circle indicates CgGS, square indicates EcGS, triangle indicates HpGS, and inverted triangle indicates TcGS. Data for the three technical replicates were plotted, and the bars indicate the mean.

FIG. 32: CLUSTAL O(1.2.4) multiple sequence alignment of GS candidates (SEQ ID NO: 15, 2, 13, 14, respectively). The sequence of the adenylation loop is shown in bold. This sequence motif is absent in TcGS. In addition, the conserved tyrosine residue (Y, underlined) that is adenylated in CgGS and EcGS is replaced with a phenylalanine (F, underlined) in HpGS.

FIG. 33: CLUSTAL O(1.2.4) multiple sequence alignment of ArgZ candidates (SEQ ID NO: 8, 3, 9, 7, respectively). The sequence of the motif defining arginine dihydrolase activity is shown in bold, with the critical asparagine residue also underlined.

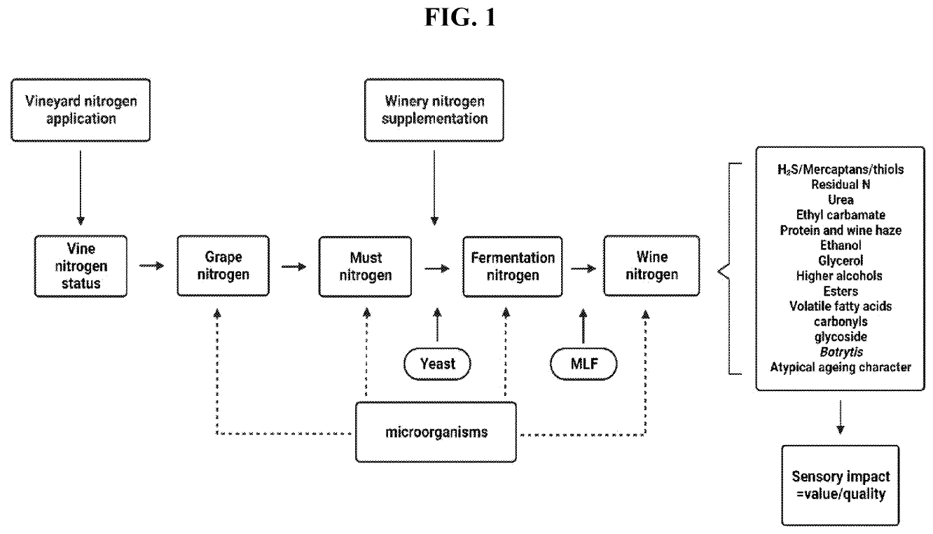

### DETAILED DESCRIPTION

The following description sets forth numerous aspects, configurations, options, parameters, and the like. It should be recognised, however, that such description is not intended as a limitation on the scope of the present disclosure; it is instead provided as a description of exemplary embodiments.

### Definitions

Throughout the specification and claims, the following terms take the meanings explicitly associated herein, unless the context clearly dictates otherwise.

The various examples, embodiments, and aspects as set out herein may be readily combined, without departing from the scope or spirit of this disclosure. Thus, the phrase “in one example”, “in one embodiment”, or “in one aspect” is not necessarily exclusive of other examples, embodiments, or aspects that are also described. In the same way, the phrase “in another example”, “in another embodiment”, or “in another aspect” is not necessarily exclusive of other examples, embodiments, or aspects that are described.

In each instance herein, in descriptions, embodiments, and examples of the present disclosure, the terms “comprising”, “including”, etc, are to be read expansively, without limitation. Thus, unless the context clearly requires otherwise, throughout the description and the claims, the words “comprise”, “comprising”, and the like are to be construed in an inclusive sense as to opposed to an exclusive sense, that is to say in the sense of “including but not limited to”.

Where a range is given in the specification, for example, a temperature range, a time range, or a composition range, all intermediate ranges and subranges, as well as all individual values included in the ranges given are intended to be included in the disclosure. Thus, each range that is specified (e.g., 1 to 10) includes all possible combinations of numerical values between the lowest value and the highest value enumerated (e.g., 1, 1.1, 2, 3, 3.3, 4, 5.5, 6, 7, 8.9, 9 and 10) and also any range of rational numbers within that range (e.g., 2 to 8, 1.5 to 5.5, and 3.1 to 4.9), and, therefore, all sub-ranges of all ranges expressly disclosed herein are hereby expressly disclosed. The numeric values provided in parentheses here are only examples of what is specifically intended and all possible combinations of numerical value between the lowest value and the highest value enumerated are to be considered to be expressly stated in this disclosure in a similar manner.

As used herein “and/or” means additionally or alternatively.

As used herein “based on” is not exclusive and allows for being based on additional factors not described, unless the context clearly dictates otherwise. The meaning of “in” includes “in” and “on.”

Any use of a term in the singular also encompasses plural forms. Thus, throughout the specification, the meaning of “a”, “an”, and “the” include plural references.

The term “about” or “approximately” means up to 10% greater than or up to 10% lesser than a particular value.

As used herein, an “isolated” component (e.g., isolated peptide, polypeptide, or enzyme) refers to a component that has been purified from (e.g., separated from) other components. An isolated component may have: about 70% purity or greater, about 80% purity or greater, about 90% purity or greater; or, in particular aspects, about 99% purity or greater. An isolated component may be obtained by any standard method or combination of methods, including biochemical, recombinant, and synthetic techniques.

“Isolated” as used herein with reference to polynucleotide, peptide, or polypeptide sequences describes a sequence that has been removed from its originating environment, e.g., natural cellular environment or synthetic environment. The polynucleotide, peptide, or polypeptide sequences of this disclosure may be prepared by at least one purification step.

“Isolated” when used herein in reference to a cell or host cell describes to a cell or host cell that has been obtained or removed from an organism or from its natural environment and is subsequently maintained in a laboratory environment. The term encompasses single cells, per se, as well as cells or host cells comprised in a cell culture and can include a single cell or single host cell.

In each instance herein, in descriptions, embodiments, and examples of the present disclosure, the terms “comprising”, “including”, etc., are to be read expansively, without limitation. Thus, unless the context clearly requires otherwise, throughout the description and the claims, the words “comprise”, “comprising”, and the like are to be construed in an inclusive sense as to opposed to an exclusive sense, that is to say in the sense of “including but not limited to”.

The term “biological sample” can be any material taken from an organism. Exemplification include body fluid or other matter obtained from a subject (e.g., animal, mammal, or human), material derived from an organism, or a sample that has organisms in it. Also included are certain tissues, for example, tumour tissue or nervous tissue, or body fluid such as blood, urine, sputum, saliva, mucus, vitreal fluid, synovial fluid, semen, cerebrospinal fluid, lymph fluid, bone marrow, amniotic fluid, bile, lacrimal fluid, perspiration, etc. Specifically noted as biological samples are blood samples (e.g., blood serum or plasma), urine samples, saliva samples, cerebrospinal fluid samples, lymph fluid samples, eukaryotic cell culture samples, and bacterial cell culture samples. Other biological samples are noted herein.

The term “construct”, e.g., “genetic construct”, refers to a polynucleotide molecule, usually double-stranded DNA, which may have cloned or inserted into it another polynucleotide molecule. For example, a construct may have an unidentified polynucleotide insert that is prepared from an environmental sample or as a cDNA, but not limited thereto. A construct may contain the elements that permit transcription of a cloned or inserted polynucleotide molecule, and, optionally, for translating the transcript into a polypeptide. The inserted polynucleotide molecule may be derived from the host cell, or may be derived from a different cell or organism. Once inside the host cell the construct may become integrated in the host chromosomal DNA. The construct may be linked to a vector.

The term “vector” as used herein refers to a polynucleotide molecule, usually double stranded DNA, which is used to replicate or express a construct. The vector may be used to transport a construct into a given host cell.

The term “polynucleotide(s),” as used herein, means a single or double-stranded deoxyribonucleotide or ribonucleotide polymer of any length, and include as non-limiting examples, coding and non-coding sequences of a gene, genomic DNA, recombinant polynucleotides, isolated and purified naturally occurring DNA or RNA sequences, synthetic RNA and DNA sequences, fragments, constructs, vectors and modified polynucleotides. Reference to nucleic acids, nucleic acid molecules, nucleotide sequences, and polynucleotide sequences is to be similarly understood.

The term “polypeptide”, as used herein, encompasses amino acid chains of any length, wherein the amino acid residues are linked by covalent peptide bonds. “Polypeptide” may refer to a polypeptide that is a purified natural product, or that has been produced partially or wholly using recombinant or synthetic techniques. The term may refer to an aggregate of a polypeptide such as a dimer or other multimer, a fusion polypeptide, a polypeptide fragment, a polypeptide variant, fragment, or derivative thereof. The term “polypeptide” is used interchangeably herein with the terms “peptide” and “protein”. The term, a “polypeptide” may also refer to an “enzyme”.

A “fragment” of a polypeptide is a subsequence of a polypeptide. In certain aspects, the fragment is a functional fragment. A functional fragment performs a function that is required for a biological activity or binding and/or provides three dimensional structure of the polypeptide. The term may refer to a polypeptide fragment, an aggregate of a polypeptide fragment, a fusion polypeptide fragment, a fragment of a polypeptide variant, or a fragment of a polypeptide derivative thereof that is capable of performing the polypeptide activity.

The term “full length” as used herein with reference to a wild-type polypeptide sequence means a polypeptide that comprises a contiguous sequence of amino acid residues where each amino acid residue has been expressed from each of its corresponding codons in the polynucleotide over the entire length of the coding region and resulting in a fully functional polypeptide, peptide or protein. As will be appreciated by a person of ordinary skill in the field, a “full length” polypeptide contains the amino acid sequence that corresponds to and has been expressed from each and every codon encoded by the polynucleotide comprising the entire coding region of the polypeptide, wherein each of said codons is located between the start codon and the termination codon normally associated with that coding region.

The term “expressing” refers to the expression of a nucleic acid transcript from a nucleic acid template and/or the translation of that transcript into a polypeptide, and is used herein as commonly used in the art.

The term “incubating” refers to the placing together of elements so they may interact and is used herein as commonly used in the art.

The term “endogenous” as used herein refers to a constituent of a cell, tissue or organism that originates or is produced naturally within that cell, tissue or organism. An “endogenous” constituent may be any constituent including but not limited to a polynucleotide, a polypeptide, or a peptide.

The term “exogenous” as used herein refers to any constituent of a cell, tissue or organism that does not originate or is not produced naturally within that cell, tissue or organism. An exogenous constituent may be, for example, a polynucleotide sequence that has been introduced into a cell, tissue or organism, or a polypeptide expressed in that cell, tissue or organism from that polynucleotide sequence.

“Naturally occurring” as used herein with reference to a polynucleotide sequence according to this disclosure refers to a primary polynucleotide sequence that is found in nature. A synthetic polynucleotide sequence that is identical to a wild polynucleotide sequence is, for the purposes of this disclosure, considered a naturally occurring sequence. What is important for a naturally occurring polynucleotide sequence is that the actual sequence of nucleotide bases that comprise the polynucleotide is found or known from nature. For example, a wild-type polynucleotide sequence is a naturally occurring polynucleotide sequence, but not limited thereto. A naturally occurring polynucleotide sequence also refers to a variant polynucleotide sequence as found in nature that differs from wild-type. For example, allelic variants and naturally occurring recombinant polynucleotide sequences due to hybridisation or horizontal gene transfer, but not limited thereto.

“Non-naturally occurring” as used herein with reference to a polynucleotide sequence according to this disclosure refers to a polynucleotide sequence that is not found in nature. Examples of non-naturally occurring polynucleotide sequences include artificially produced mutant and variant polynucleotide sequences, made for example by point mutation, insertion, or deletion, but not limited thereto. Non-naturally occurring polynucleotide sequences also include chemically evolved sequences. What is important for a non-naturally occurring polynucleotide sequence is that the actual sequence of nucleotide bases that comprise the polynucleotide is not found or known from nature.

The term, “wild-type” when used herein with reference to a polynucleotide refers to a naturally occurring, non-mutant form of a polynucleotide, polypeptide, or organism. A mutant polynucleotide means a polynucleotide that has sustained a mutation, including one or more of a point mutation, insertion, deletion, substitution, amplification, or translocation, but not limited thereto. A mutant polypeptide means a polypeptide that includes a mutation, including one or more of an insertion, deletion, substitution, but not limited thereto. A wild-type polypeptide may be expressed from a wild-type polynucleotide, or from a mutant polynucleotide.

“Homologous” as used herein with reference to polynucleotide regulatory elements, means a polynucleotide regulatory element that is a native and naturally-occurring polynucleotide regulatory element. A homologous polynucleotide regulatory element may be operably linked to a polynucleotide of interest such that the polynucleotide of interest can be expressed from a, vector, construct, or expression cassette according to this disclosure.

“Heterologous” as used herein with reference to polynucleotide regulatory elements, means a polynucleotide regulatory element that is not a native and naturally-occurring polynucleotide regulatory element. A heterologous polynucleotide regulatory element is not normally associated with the coding sequence to which it is operably linked. A heterologous regulatory element may be operably linked to a polynucleotide of interest such that the polynucleotide of interest can be expressed from a vector, construct, or expression cassette according to this disclosure. Such promoters may include promoters normally associated with other genes, ORFs or coding regions, and/or promoters isolated from any other bacterial, viral, eukaryotic, or mammalian cell.

“Isolated” as used herein with reference to polynucleotide or polypeptide sequences describes a sequence that has been removed from its natural cellular environment. An isolated molecule may be obtained by any standard method or combination of methods, including biochemical, recombinant, and synthetic techniques. The polynucleotide or polypeptide sequences may be prepared by at least one purification step.

“Isolated” when used herein in reference to a cell or host cell describes to a cell or host cell that has been obtained or removed from an organism or from its natural environment and is subsequently maintained in a laboratory environment. The term encompasses single cells, per se, as well as cells or host cells comprised in a cell culture and can include a single cell or single host cell.

The term “recombinant” refers to a polynucleotide sequence that is removed from sequences that surround it in its natural context and/or is recombined with sequences that are not present in its natural context. A “recombinant” polypeptide sequence is produced by translation from a “recombinant” polynucleotide sequence.

The term “modified” refers to a component that is not a naturally occurring component. In the same way, a “modified polypeptide” or “modified enzyme” refers to a polypeptide that is not a naturally occurring polypeptide. Modification may be carried out in accordance with the disclosed methods, e.g., sequence variation or other modification, such as post-translational modifications. For example, various methods of recombination may be used to achieve modification. Modified enzymes and polypeptides useful in this disclosure may have biological activities that are the same or similar to those of a corresponding wild-type molecule i.e., functional modifications. Alternatively, modified enzymes and polypeptides may have biological activities that differ from their corresponding wild-type molecules. In certain embodiments, the differences are altered activity and/or binding specificity. For example, a functional modification may produce a particular type of product. In certain embodiments, the levels of product produced by the functional modification may be higher or lower than produced by the wild-type molecule. In particular embodiments, a modified enzyme may comprise a recombinant enzyme, a modified polypeptide may comprise a recombinant polypeptide, and a modified enzyme module may comprise a recombinant enzyme module, as set out in this description.

As used herein, the term “variant” refers to polynucleotide or polypeptide sequences different from the specifically identified sequences, wherein one or more nucleotides or amino acid residues is deleted, substituted, or added. Variants may be naturally occurring allelic variants, or non-naturally occurring variants. Variants may be produced to include one or more sequence tags, e.g., affinity tags such as those described herein. Variants may be from the same or from other species and may encompass homologues, paralogues, and orthologues. In certain embodiments, variants of the polypeptides have biological activities that are the same or similar to those of a corresponding wild-type molecule; i.e., functional variants of the parent polypeptide or polynucleotide. In certain embodiments, variants of the polypeptides have biological activities that differ from their corresponding wild-type molecules. In certain embodiments, the levels of activity of a functional variant may be higher or lower than produced by the wild-type enzyme.

The term “variant” with reference to polynucleotides and polypeptides encompasses all forms of polynucleotides and polypeptides as defined herein. In particular embodiments, a variant polypeptide, including an enzyme variant, as described herein will retain functionality of the reference polypeptide, for example, enzymatic activity will be retained.

As used herein, the term “mutagenesis” refers to methods to alter a polynucleotide sequence either in vitro or in vivo, most commonly to change the sequence of one or more polypeptides encoded therein. Mutagenesis methods include as non-limiting examples, site-directed mutagenesis, de novo synthesis of sequences carrying mutations, error-prone PCR, DNA shuffling, chemical mutagenesis, application of ultraviolet radiation, genome shuffling, and use of mutator strains.

As used herein, the term in vitro refers to a reaction performed outside of the confines of a living cell or a host organism.

As used herein, the term in vivo refers to a reaction performed within a living cell and/or within a host organism.

“Activation” refers to any enzymatic modification or action that causes the substrate of a given enzyme to adopt a functional conformation or perform a functional role that the substrate was not capable of performing before being activated.

The term “reporter product” as used herein refers to a detectable product formed due to the activity of an activated enzyme.

As used herein, “protein”, “polypeptide” and “enzyme” mean the same thing and are used interchangeably.

The term “BpsA” (used on its own) or “BpsA polypeptide” refer to an enzyme (e.g., SEQ ID NO: 1) capable of converting glutamine to indigoidine. A preferred variant of a BpsA polypeptide is capable of converting glutamine to indigoidine and shares at least 70% sequence identity with a reference BpsA polypeptide (e.g., SEQ ID NO: 1), or at least 75%, or at least 80%, or at least 85%, or at least 90%, or at least 95%, or at least 96%, or at least 97%, or at least 98%, or at least 99% sequence identity. Naturally occurring or non-naturally occurring BpsA polypeptides may be utilised in accordance with this disclosure.

The term “GS” (used on its own), “glutamine synthetase” (used on its own) “GS polypeptide” refer to an enzyme (e.g., SEQ ID NO: 2 or SEQ ID NO: 13-14) capable of converting glutamate and ammonium into glutamine. A preferred variant of a GS polypeptide is capable of capable of converting glutamate and ammonium into glutamine and shares at least 70% sequence identity with a reference GS polypeptide (e.g., SEQ ID NO: 2 or SEQ ID NO: 13-14), or at least 75%, or at least 80%, or at least 85%, or at least 90%, or at least 95%, or at least 96%, or at least 97%, or at least 98%, or at least 99% sequence identity. Naturally occurring or non-naturally occurring GS polypeptides may be utilised in accordance with this disclosure.

The term “ArgZ” (used on its own), “arginine dihydrolase” (used on its own) “ArgZ polypeptide” refer to an enzyme (e.g., SEQ ID NO: 3 or SEQ ID NO: 7-9) capable of converting arginine to ornithine and ammonium. A preferred variant of a ArgZ polypeptide is capable converting arginine to ornithine and ammonium and shares at least 70% sequence identity with a reference ArgZ polypeptide (e.g., SEQ ID NO: 3 or SEQ ID NO: 7-9), or at least 75%, or at least 80%, or at least 85%, or at least 90%, or at least 95%, or at least 96%, or at least 97%, or at least 98%, or at least 99% sequence identity. Naturally occurring or non-naturally occurring ArgZ polypeptides may be utilised in accordance with this disclosure.

It is understood that, for any DNA molecule disclosed herein, the corresponding RNA molecule and peptide/polypeptide molecules are also encompassed and disclosed. Likewise, for any peptide/polypeptide molecule disclosed herein, the corresponding RNA and DNA sequences are also considered to be encompassed and disclosed. In addition, where there are multiple sequence identifiers, e.g., “SEQ ID NO: 1-3”, this format may be understood as referring to each sequence individually, or any combination thereof.

### Biosensors and Biological Sensing Methods

According to Thakur & Ragavan (2013), biosensors are quantitative analytical tools that contain a biological sensing element with a transducer that can detect and produce a signal from an analyte. They are used in various fields for their easy, rapid, and inexpensive measurement. Enzymes, antibodies, and DNA are typically immobilised and used as the biorecognition element due to their selectivity (Kawamura & Miyata, 2016). Once recognition occurs, a signal can be transduced into different indicators such as electrical or optical by electrochemical or colorimetric techniques. There are different types of biosensors which can be identified by their recognition element and transducer (Mehrotra, 2016).

An optical enzyme-based biosensor is principally composed of an enzyme and an optical transducer. Enzymes, as the biological sensing element, have widely been used in the biosensor industry due to their high specificity and selectivity for their substrate as well as their catalytic properties. The optical transducer, which provides a measurable signal, is often based on absorption/reflectance, luminescence, chemiluminescence, or evanescent waves (Nguyen et al., 2019; Zhu et al., 2019).

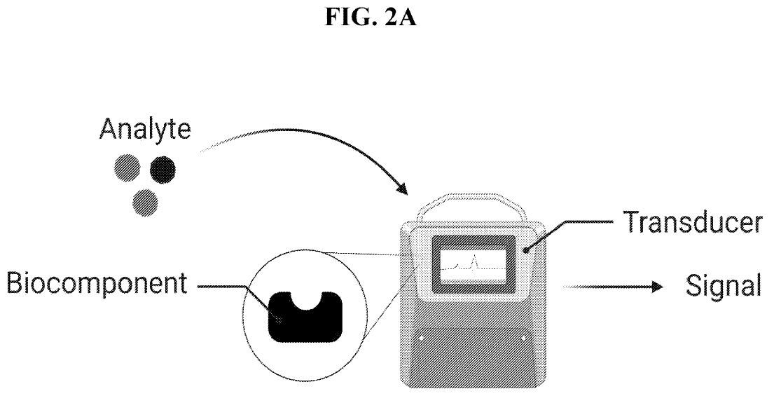

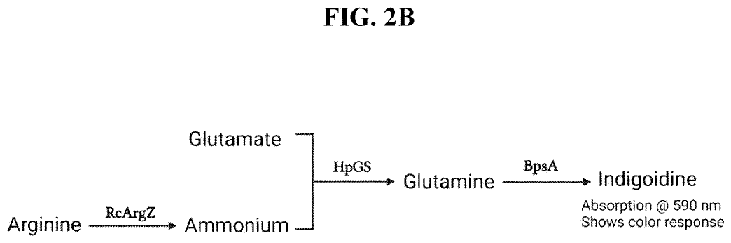

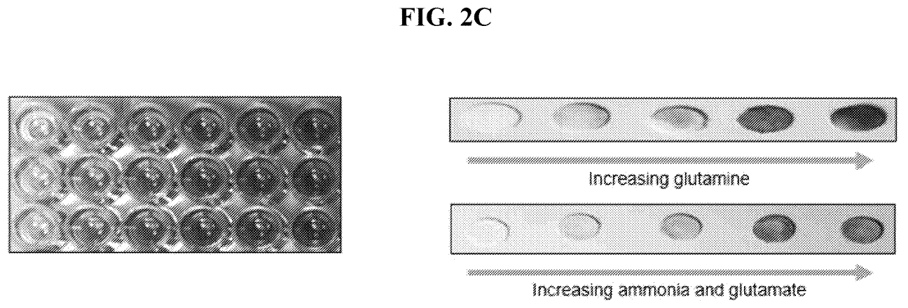

The goal of the present research has been to construct two-enzyme and three-enzyme biosensor systems. The biosensor systems can be used, for example, to quantify yeast assimilable nitrogen, including key YAN constituents, in grape samples (e.g., juice from grape must) with a straightforward colorimetric readout. Other types of testing may also be utilised. The production of indigoidine by blue pigment synthetase A (BpsA) has been employed as the colorimetric readout for the biosensor. BpsA has been used in conjunction with one or two other enzymes. As an exemplification, the three-enzyme system can sense and report the combined levels of arginine, glutamine, ammonium, with these being major constituents of yeast assimilable nitrogen (YAN). FIG. 2B illustrates a schematic diagram of three-enzyme biosensor system and its components in accordance with one embodiment of this disclosure.

### Pigment Producing Enzyme

Blue-pigment indigoidine synthetase (BpsA) is a single-module non-ribosomal-peptide synthetase enzyme (NRPS). It converts two molecules of L-glutamine into one molecule of indigodine (Owen et al., 2011). Brown et al. (2017) developed this enzyme into a biosensor for detecting glutamine levels in human biological samples.

BpsA consists of a single module comprising four domains (an Adenylation (A-) domain, an Oxidation (Ox-) domain, a Thiolation (T-) domain and a Thioesterase (TE-) domain. Collectively, these work to convert two molecules of L-glutamine at a time, via an ATP powered reaction, into a blue pigment that has been identified as water-insoluble 3,3′-bipyridyl, known as indigoidine (Kuhn et al., 1965; Mortimer et al., 1966; Takahashi et al., 2007):

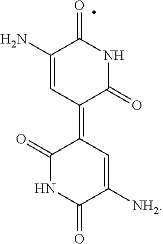 [SMILES: NC1=C/C(=C2/C=C(N)C(=O)NC2=O)C(=O)NC1=O]

The blue pigment indigoidine absorbs light at 590 nm, which can be quantitated spectroscopically (Yumusak et al., 2019). Noted in particular is Streptomyces lavendulae BpsA (S/BpsA). See, e.g., WO 2015/084189, which is incorporated by reference herein, in its entirety.

BpsA displays significant amino acid similarity to IndC (Reverchon et al., 2002) (GenBank™ accession number CAB87990) from Erwinia chrysanthemi (57% identity) and IgiD (GenBank™ accession number AAD54007) from Vogesella indigofera (46% identity) (Takahashi et al., 2007). Other NRPS with sequence similarity include EpoB (GenBank™ accession number AAF62881), EpoP (GenBank™ accession number AAF26925), MtaC (GenBank™ accession number AAF19811), MtaD (GenBank™ accession number AAF19812), and Blm (GenBank™ accession number AAG02365) (Takahashi et al., 2007).

All of these enzymes show highly conserved amino acid sequences in the Ox domain (Takahashi et al., 2007). In particular, two key sequence motifs have been identified in the Ox domain: motif 1 K-Y/F-G/R/T/Q/A-Y-A/P/G-S-A/P-G-G/A/S-L/I/T/S-Y-P/A/G-V/T-Q (SEQ ID NO: 21); and motif 2 G-Y/F/H/A/T/S-Y/H-Y-Y/V-H/Q/D-P-V/F/L/K/A-E/R/H/Q-H/N-R/Q/T/E-L (SEQ ID NO: 22) (Takahashi et al., 2007).

In addition to the noted polypeptides, there are numerous pigment producing enzymes to select from. By kinetic and optimisation experiments, the inventors have identified BpsA as having particular utility with the disclosed methods.

### Ammonium Sensing Enzyme

Glutamine synthetase (GS, EC 6.3.1.2), an enzyme present in all organisms, is a key enzyme involved with nitrogen metabolism and glutamine biosynthesis (Shapiro & Stadman, 1970). There are three different classes of GS enzymes, which will be further discussed below. GS catalyses the ATP-dependent conversion of glutamate and ammonium into glutamine in the presence of divalent cations: Mn²⁺ or Mg²⁺ (Eisenberg et al., 2000; Joo et al., 2018). However, according to Stadman (2004), GS activity is strictly controlled due to its central role in nitrogen metabolism.

The three GS classes were identified as GS-I, GS-II, and GS-III. Though the three classes vary in sequence similarity they are, nonetheless, structurally related (Berges et al., 2008).

GS-I class I enzymes, represented by GlnA, are commonly found in prokaryotes and archaea (Robertson et al., 1999) and some vascular plants (Mathis et al., 2000). This class of enzyme has 12 identical subunits arranged in two hexagonal rings. As stated by Brown et al. (1993), GS-I is subdivided into two isozymes: GS-Iα and GS-Iβ. GS-Iα is normally found in low G+C Gram-positive bacteria, thermophilic bacteria, and euryarchaeota, whereas GS-Iβ is found in other bacteria. The key difference between the two isozymes is that GS-Iα is feedback inhibited by the end product of glutamine metabolism while GS-Iβ is controlled by post-translational modification. Furthermore, GS-Iβ has a specific 25 amino acid residues that are not found in GS-la.

Type II GS enzymes are represented by GlnII. This class of enzymes is found in eukaryotes and a few bacteria such as the Rhizobiaceae, Frankiaceae, and Streptomycetaceae families (these bacteria also contain GS-I) (Robertson et al., 2001). This class of enzyme consists of eight uniformly sized subunits. A study made by Ghoshroy et al. (2010) indicates two isozymes of GS-II were found in plants with one located in the chloroplast.

GS-III class enzymes, which are represented by GlnN, are found in some bacteria, cyanobacteria, and archaea (Herrero et al., 2001). This class of enzyme consists of six identical subunits. According to Rooyen et al. (2011), GS-III is the most divergent type, having less than 10% sequence identity with Type I and Type II, and has been less studied.

Therefore, there are numerous candidates to assess to identify an appropriate ammonium sensing enzyme. By detailed data mining, kinetic experiments, and optimisation experiments, the inventors have identified H. pylori glutamine synthetase (HpGS) as having particular utility with the disclosed methods.

### Arginine Metabolising Enzyme

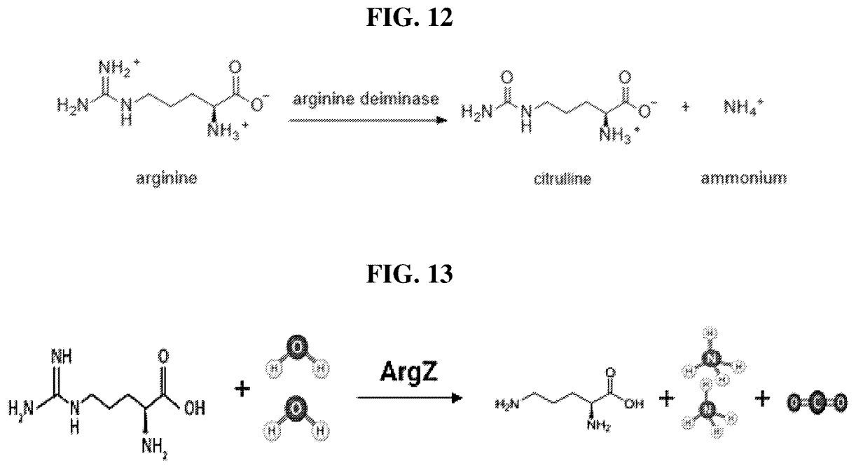

Arginine deiminase (ADI, Enzyme Commission classification 3.5.3.6) belongs to a group of guanidine-modifying enzymes (Galkin et al., 2004). It catalyses the irreversible deamination of arginine resulting in the formation of citrulline and ammonium (FIG. 12) (Lu, Galkin, Herzberg, & Dunaway-Mariano, 2004). ADI has been an enzyme of interest for biosensors, where it has been used to detect the level of arginine in beverages concerning ethyl carbamate production (Stasyuk et al., 2016; Verma, Singh, & Kaur, 2015).

Arginine dihydrolase (ArgZ) is a newly discovered enzyme that is involved in the ornithine-ammonium cycle in cyanobacteria (Zhang et al., 2018). Its catalytic mechanism involves two successive amine hydrolysis reactions (Zhuang et al., 2020). ArgZ catalyses the conversion of arginine to ornithine, two molecules of ammonium, and one molecule of carbon dioxide (FIG. 13).

Arginine deiminase and arginine dihydrolase are both enzymes for arginine metabolism. Structural studies on both enzymes indicate the same catalytic triad of “Cys-His-Glu” (Galkin et al., 2004; Zhuang et al., 2020). However, their catalytic mechanism is different. Arginine deiminase catalyses one amine hydrolysis while arginine dihydrolase catalyses two cycles of amine hydrolysis. The determinant of the number of amine hydrolysis reactions is the amino acid that forms a hydrogen bond with the guanidinium moiety of arginine.

Arginine dihydrolase contains an asparagine while arginine deiminase contains an aspartate at the corresponding position. The aspartate in arginine deiminase creates two strong salt bridge bonds with the guanidinium moiety of arginine. The interaction enhances the stability and therefore allows water to diffuse and bind at the active centre once one amine is released, completing one round of amine hydrolysis. On the other hand, the presence of asparagine poses less restraint allowing the rotation of the second amine after the release of the first, which lets the second water molecule diffuse in the active centre resulting in the second cycle of amine hydrolysis.

There are many different types of arginine deiminase and arginine dihydrolase enzymes in different organisms and cell types. Accordingly, there are numerous candidates to assess to identify an appropriate arginine metabolising enzyme. By detailed data mining, kinetic experiments, and optimisation experiments, the inventors have identified R. centenum arginine dihydrolase (RcArgZ) as having particular utility with the disclosed methods.

Accordingly, the present disclosure encompasses the combined use of BpsA and glutamine synthetase (GS) in biological sensing methods. Encompassed also is the combined use of BpsA, arginine dihydrolase (ArgZ), and glutamine synthetase (GS) in biological sensing methods. Particularly noted are RcArgZ, HpGS, and S/BpsA.

Biosensor Enzymes and their Variants

The inventors have identified and demonstrated the utility of a three-enzyme biosensor system comprising a blue-pigment indigoidine synthetase enzyme (BpsA, e.g., SEQ ID NO: 1), a glutamine synthetase (GS, e.g., SEQ ID NO: 2 or 13-14), and an arginine dihydrolase (ArgZ, e.g., SEQ ID NO: 3 or 7-9). Also identified and demonstrated herein is a two-enzyme biosensor system comprising a blue-pigment indigoidine synthetase enzyme (BpsA, e.g., SEQ ID NO: 1) and a glutamine synthetase (GS, e.g., SEQ ID NO: 2 or 13-14). In specific aspects, this disclosure encompasses combinations of these polypeptides and methods of preparing and using this combination of polypeptides. Also encompassed are variant polypeptides and polynucleotides and their methods of preparation and use.

Therefore, in addition to the sequences noted herein, the methods of this disclosure may be used to obtain modified polypeptide and polynucleotide sequences. In one embodiment, the present disclosure utilises modified enzymes, for example, fragments or sequence variations as described herein. Modifications of BpsA, GS, and ArgZ polypeptides, including modified domains, and modified modules, are specifically encompassed by the present disclosure. Modified polynucleotides encoding these sequences are also encompassed.

Therefore, in addition the BpsA, GS, and ArgZ nucleotide sequences set out herein, variant sequences may also be utilised. In various embodiments, nucleic acid variants encompass recombinantly and synthetically produced nucleic acids.

As exemplifications, variant polynucleotide sequences exhibit at least 50%, at least 60%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, and at least 99% identity to a sequence of the present disclosure (e.g., any one of SEQ ID NO: 4-6 or any one of SEQ ID NO: 10-12).

In the same way, a nucleotide sequence for a BpsA, GS, and ArgZ polypeptide, or a module, or a domain thereof, may be modified to include the above noted levels of sequence identity.

As a variant polynucleotide sequence, a fragment of a polynucleotide sequence includes a subsequence of contiguous nucleotides. In one embodiment, the polynucleotide fragment allows expression of at least a portion of a polypeptide, e.g., expression of one or more functional domain of the polypeptide.

Variant polynucleotides include polynucleotides that differ from the disclosed sequences but that, as a consequence of the degeneracy of the genetic code, encode a polypeptide having similar activity to a polypeptide encoded by a disclosed polynucleotide. A sequence alteration that does not change the amino acid sequence of the polypeptide is termed a silent variation. Except for ATG (methionine) and TGG (tryptophan), other codons for the same amino acid may be changed by art recognised techniques, e.g., to optimise codon expression in a particular host organism.

For BpsA polynucleotides, sequence identity may be found over a comparison window of at least 1500 nucleotide positions, at least 2000 nucleotide positions, at least 2500 nucleotide positions, at least 3000 nucleotide positions, at least 3500 nucleotide positions, at least 3800 nucleotide positions, or over the entire length of a polynucleotide used according to a method of this disclosure.

For GS polynucleotides, sequence identity may be found over a comparison window of at least 900 nucleotide positions, at least 1000 nucleotide positions, at least 1100 nucleotide positions, at least 1200 nucleotide positions, at least 1300 nucleotide positions, at least 1400 nucleotide positions, or over the entire length of a polynucleotide used according to a method of this disclosure.

For ArgZ polynucleotides, sequence identity may be found over a comparison window of at least 650 nucleotide positions, at least 700 nucleotide positions, at least 750 nucleotide positions, at least 800 nucleotide positions, at least 850 nucleotide positions, at least 900 nucleotide positions, or over the entire length of a polynucleotide used according to a method of this disclosure.

For a polynucleotide encoding a module or domain of a polypeptide, shorter regions may be compared, for example, at least 50 nucleotide positions, at least 100 nucleotide positions, at least 200 nucleotide positions, at least 300 nucleotide positions, at least 400 nucleotide positions, at least 500 nucleotide positions, at least 600 nucleotide positions, at least 700 nucleotide positions, at least 800 nucleotide positions, at least 900 nucleotide positions, or at least 1000 nucleotide positions.

Polynucleotide sequence alterations resulting in conservative substitutions of one or several amino acids in the encoded polypeptide sequence without significantly altering its biological activity are also included in this disclosure. A skilled artisan will be aware of methods for making phenotypically silent amino acid substitutions (see, e.g., Bowie et al. 1990).

Polynucleotide sequence identity and similarity can be determined in the following manner. The subject polynucleotide sequence is compared to a candidate polynucleotide sequence using sequence alignment algorithms and sequence similarity search tools such as in GenBank, EMBL, Swiss-PROT and other databases. Nucleic Acids Res 29:1-10 and 11-16, 2001 provides examples of online resources.

In addition to the particular BpsA, GS, and ArgZ polypeptides set out herein, variant sequences may also be utilised. In various embodiments, polypeptide variants encompass recombinantly and synthetically produced polypeptides.

As exemplifications, variant polypeptide sequences exhibit at least 50%, at least 60%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a sequence of the present disclosure (e.g., any one of SEQ ID NO: 1-3 or SEQ ID NO: 7-9 or SEQ ID NO: 13-14).

In the same way, an amino acid sequence of a BpsA, GS, and ArgZ polypeptide, or a module, or a domain thereof, may be modified to include the above noted levels of sequence identity.

As a variant polypeptide sequence, a fragment of a polypeptide sequence includes a subsequence of contiguous amino acids. In one embodiment, a polypeptide fragment is a functional fragment, i.e., a fragment capable of binding or other biological activity. For example, a polypeptide fragment may be capable of producing a particular substrate. In a particular embodiment, the polypeptide fragment may include at least one functional domain. For example, for an BpsA polypeptide, a fragment would include an A domain, an A domain fragment, or a modification thereof.

As to polypeptide variants, an amino acid sequence may differ from a polypeptide disclosed herein by one or more conservative amino acid substitutions, deletions, additions or insertions which do not affect the biological activity of the peptide. Conservative substitutions typically include the substitution of one amino acid for another with similar characteristics, e.g., substitutions within the following groups: glycine, alanine; valine, isoleucine, leucine; aspartic acid, glutamic acid; asparagine, glutamine; serine, threonine; lysine, arginine; and phenylalanine, tyrosine. Non-conservative substitutions will entail exchanging a member of one of these classes for a member of another class.

Other variants include peptides with modifications which influence peptide stability. Such analogues may contain, for example, one or more non-peptide bonds (which replace the peptide bonds) in the peptide sequence. Also included are analogues that include residues other than naturally occurring L-amino acids, e.g. D-amino acids or non-naturally occurring synthetic amino acids, e.g. beta or gamma amino acids and cyclic analogues.

Substitutions, deletions, additions, or insertions may be made by standard mutagenesis methods. A skilled worker will be aware of methods for making phenotypically silent amino acid substitutions. See, for example, Bowie et al. 1990. A polypeptide may be modified during or after synthesis, for example, by biotinylation, benzylation, glycosylation, phosphorylation, amidation, by derivatisation using blocking/protecting groups and the like. Such modifications may increase stability or activity of the polypeptide.

For BpsA polypeptides, sequence identity may be found over a comparison window of at least 600 amino acid positions, at least 700 amino acid positions, at least 800 amino acid positions, at least 900 amino acid positions, at least 1000 amino acid positions, at least 1100 amino acid positions, at least 1200 amino acid positions, or over the entire length of a polypeptide used in or identified according to a method of this disclosure.

For GS polypeptides, sequence identity may be found over a comparison window of at least 300 amino acid positions, at least 350 amino acid positions, at least 300 amino acid positions, at least 350 amino acid positions, at least 400 amino acid positions, at least 450 amino acid positions, or over the entire length of a polypeptide used according to a method of this disclosure.

For ArgZ polypeptides, sequence identity may be found over a comparison window of at least 100 amino acid positions, at least 120 amino acid positions, at least 175 amino acid positions, at least 200 amino acid positions, at least 220 amino acid positions, at least 250 amino acid positions, or over the entire length of a polypeptide used according to a method of this disclosure.

For a polypeptide module or domain, shorter regions may be compared, for example, at least 8 amino acid positions, at least 10 amino acid positions, at least 20 amino acid positions, at least 30 amino acid positions, at least 40 amino acid positions, at least 50 amino acid positions, at least 60 amino acid positions, at least 70 amino acid positions, at least 80 amino acid positions, at least 90 amino acid positions, or at least 100 amino acid positions.

Polypeptide variants also encompass those that exhibit a similarity to one or more of the specifically identified sequences that is likely to preserve the functional equivalence of those sequences and which could not reasonably be expected to have occurred by random chance. For polynucleotides and polypeptides, exemplary sequence alignment platforms include but are not limited to: homology alignment algorithms (Needleman and Wunsch (1970) J Mol Biol 48: 443); local homology algorithms (Smith and Waterman (1981) Adv Appl Math 2: 482); searches for similarity (Pearson and Lipman (1988) PNAS USA 85: 2444).

In specific embodiments, the BLAST algorithm may be used (Altschul et al. (1990) J Mol Biol 215: 403-410; Henikoff and Henikoff. (1989) PNAS USA 89: 10915; Karlin and Altschul (1993) PNAS USA 90: 5873-5787). Software for performing BLAST analyses is publicly available through the National Center for Biotechnology Information. Other examples of alignment software include GAP, BESTFIT, FASTA, PILEUP, and TFASTA provided by Wisconsin Genetics Software Package (Genetics Computer Group), and CLUSTAL programs such as ClustalW, ClustalX, and Clustal Omega (see, e.g., Thompson et al. (1994) Nuc Acids Res 22: 4673-4680).

Expression of Biosensor Enzymes and their Uses

As indicated above, this disclosure encompasses a biosensor system comprising blue-pigment indigoidine synthetase enzyme (BpsA, e.g., SEQ ID NO: 1), a glutamine synthetase (GS, e.g., SEQ ID NO: 2 or 13-14), and an arginine dihydrolase (ArgZ, e.g., SEQ ID NO: 3 or 7-9). Also encompassed is a biosensor system comprising blue-pigment indigoidine synthetase enzyme (BpsA, e.g., SEQ ID NO: 1) and a glutamine synthetase (GS, e.g., SEQ ID NO: 2 or 13-14). Wild-type and variant polypeptides are encompassed in this disclosure along with corresponding preparative methods.

Therefore, in one embodiment, the BpsA, GS, or ArgZ polypeptide (e.g., a wild-type or variant polypeptide) can be expressed using a nucleic acid construct. For example, a BpsA, GS, or ArgZ construct may be used. For example, a nucleic acid expression construct can comprises a polynucleotide sequence (e.g., any one of SEQ ID NO:4-6 or 10-12) that encodes an polypeptide (e.g., any one of SEQ ID NO: 1-3 or 7-9 or 13-14) operatively linked to a promoter that allows expression of the polynucleotide sequence to form the polypeptide. The construct may produce a functional BpsA, GS, or ArgZ enzyme, or a functional variant of the BpsA, GS, or ArgZ enzyme may be produced.

An expression cassette may be used to include the elements that permit the transcription of a polynucleotide molecule that has been cloned or inserted into the construct. Optionally, the expression cassette may comprise some or all of elements for translating the transcript produced from the expression cassette into a polypeptide. An expression cassette may include BpsA, GS, or ArgZ coding regions. It may also include any noncoding regions, where desired.

The construct may be a construct for expression of the appropriate BpsA, GS, or ArgZ polypeptide, as set out herein, or any functional variants of these. The BpsA, GS, or ArgZ polynucleotide sequence may be any suitable polynucleotide sequence from any organism. In various aspects, the organism can be a bacterial cell or strain. Exemplifications include but are not limited to: Pseudomonas, Streptomyces, Helicobacter, Rhodospirillum, and Bacillus (e.g., Brevibacillus) strains, for example, Streptomyces lavendulae, Helicobacter pylori, and Rhodospirillum centenum.

The polynucleotide sequence encoding a polypeptide may be a naturally occurring (i.e., wild-type) or non-naturally occurring (e.g., modified) polynucleotide sequence. For example, a wild-type or a modified polynucleotide sequence for BpsA, GS, or ArgZ polypeptides may be used. In particular, the polynucleotide sequence encoding the BpsA, GS, or ArgZ polypeptide, or domain, or module thereof, may be a wild-type or modified polynucleotide sequence, as described herein.

In one embodiment, a construct is made by cloning a polynucleotide sequence encoding one or more wild-type or modified polypeptide as above into an appropriate vector. An appropriate vector is any vector that comprises a promoter operatively linked to the cloned, inserted polynucleotide sequence that allows expression of the one or more polypeptides from the vector. A skilled worker appreciates that different vectors may be employed in the methods of this disclosure. In addition methods for constructing vectors, including the choice of an appropriate vector, and the cloning and expression of a polynucleotide sequence inserted into an appropriate vector as described above is believed to be within the capabilities of a person of skill in the art (Sambrook et al. 2003).

In various aspects, the expressed BpsA, GS, or ArgZ polypeptide can comprise a functional polypeptide, or a functional variant thereof. Expression may be inducible, for example, with IPTG. Similar approaches may be used for the BpsA, GS, or ArgZ polypeptides disclosed herein, and any functional variants thereof. The person of skill in the art recognises that there are also many suitable alternative expression systems available that may be used in the methods of this disclosure to express the polypeptides and their variants.

Expression may be in a suitable host cell or strain, or in a cell free expression system. In one embodiment, the host cell or strain may be a cell or strain of E. coli. Particularly of interest are the BL21 AentD and BL21(DE3) Gold strains of E. coli and any variant of these strains (Pfeifer et al. 2001). Alternatively, the expression vector is chosen to allow inducible expression in a non-E. coli host cell or strain. Expression may be obtained using various in vitro expression systems, utilising standard methodology.

In one embodiment, multiple polypeptides are co-expressed in the same host cell or strain (e.g., two or more of BpsA, GS, and ArgZ polypeptides). To achieve expression within the same host cell or strain, the nucleotide sequences encoding the polypeptides may be cloned into suitable expression vectors. Suitable vectors may have the same or compatible origins of replication in order to be stably maintained in the same host cell or strain. At least one construct can encode at least one BpsA, GS, or ArgZ polypeptide, or a functional variant thereof.

In another embodiment, polynucleotide sequences encoding one or more BpsA, GS, or ArgZ polypeptides may be integrated into the chromosome of an appropriate host organism as described herein, to produce a strain useful in accordance with the present disclosure. In specific embodiments, a construct comprises a nucleotide sequence encoding a BpsA, GS, or ArgZ polypeptide, or multiple polypeptides, and a suitable regulatory promoter. One or more of these constructs may integrated into the chromosome of E. coli or other host organism in an appropriate orientation to allow expression of the polypeptide or polypeptides in the cell.

In one particular embodiment, a construct encoding a BpsA, GS, or ArgZ polypeptide is integrated into a host cell. For example, a construct may be integrated and then expressed in vivo. The constructs may allow co-expression of wild-type polypeptides or functional variants. Thus, in a specific embodiment, a construct that encodes one or more polypeptides (e.g., one or more of SEQ ID NO: 1-3 or 7-9 or 13-14) is introduced and expressed in a host cell or strain.

In particular embodiments, the present disclosure provides polynucleotide libraries that include BpsA, GS, or ArgZ nucleic acids. For example, a polynucleotide library may include at least 15, at least 25, at least 50, at least 100, at least 200, at least 300, at least 400, at least 500, at least 600, at least 700, at least 800, at least 900, or at least 1000 NRPS nucleic acids. Libraries of NRPS polynucleotides, and specifically variant BpsA, GS, or ArgZ polynucleotides, may be generated using standard methods.

As exemplifications, nucleic acid libraries may be generated to include a plurality of BpsA, GS, or ArgZ polynucleotides with modified modules or modified domains. For example, a nucleic acid library may include polynucleotides with domain substitutions, i.e., domain swap libraries. In addition, libraries of BpsA, GS, or ArgZ polynucleotides may be generated using random mutagenesis of one or more modules or domains, for example, error prone PCR may be utilised (see, e.g., Beaudry and Joyce (1992) Science 257: 635 and Bartel and Szostak (1993) Science 261: 1411). Alternative means for mutagenesis may be used, for example, chemical mutagens, radiation, amongst others. Commercial kits are also available, e.g., GeneMorph® II EZClone domain mutagenesis kit (Agilent) and Diversify™ PCR random mutagenesis kit (Clontech Laboratories, Inc). The library may be provided as a mixture of polynucleotides, or may be provided via a host cell or strain.

As one embodiment of the present disclosure, a kit is provided which includes one or more polynucleotides (e.g., any one of SEQ ID NO: 4-6 or 10-12) or one or more polypeptides (e.g., any one of SEQ ID NO: 1-3 or 7-9 or 13-14). The one or more polynucleotide or polypeptide may be a modified component as described herein. The one or more polynucleotide or polypeptide may be provided in one or more containers in the kit. The kit may employ fluid format assays or solid format assays as described herein. Additional components may also be provided with the kit, for example, one or more components to obtain expression, or one or more components to measure activity, which are intended for use with the polypeptide(s). Optionally, instructions may be provided with the kit, as well as any other item, such as any number of containers, labels, or measurement tools. The one or more polynucleotide or one or more polypeptide of the kit may be provided as isolated components, or as mixtures, or as conjugates, or as deposits (e.g., on strips, disks, dipsticks, etc), or via one or more host cells or strains, or any combination of these.

### Host Cells, Strains, and Cell Free Expression Systems

As disclosed herein, methods of production for BpsA, GS, or ArgZ polypeptide (e.g., a modified polypeptide) are provided. The expression of a BpsA, GS, or ArgZ polypeptide (e.g., a modified polypeptide) may be carried out in vitro or in vivo. In vivo expression may be carried out in a suitable host cell or strain. Host cells and their use for such production are set out in detail in this description. By use of a host cell comprising a BpsA, GS, or ArgZ polypeptide, this allows production of the enzymes and the attendant use of these enzymes.

A suitable host cell or strain may be any suitable prokaryotic or eukaryotic cell in which a polypeptide (e.g., any one of SEQ ID NO: 1-3 or 7-9 or 13-14), or any functional variants thereof, may be expressed. The suitable host cell or strain may be a bacterial cell or strain. In particular embodiments, eukaryotic cells or strains may be used. In one embodiment, the host cell or strain is a fungal or bacterial, preferably bacterial, host cell or strain. Preferably, the bacterial cell or strain is a Gram negative bacterial cell or strain. Preferably, the bacterial cell or strain is a cell or strain of E. coli. For industrial applications, the host strain may be a Bacillus (e.g., Brevibacillus), Streptomyces, Pseudomonas, Saccharomyces, or Aspergillus strain, or another bacterial or fungal strain as set out herein, or any functional variant thereof.

Introduction of a BpsA, GS, or ArgZ construct into an appropriate host cell or strain may be achieved using any of a number of available standard protocols and/or as described herein (see, e.g., Sambrook et al. 2003). Preferably, the BpsA, GS, or ArgZ construct is a construct for a polypeptide as set out herein. Preferably, the construct is inserted into an appropriate host cell or strain. Such insertion may be achieved using any of a number of available standard transformation, transduction, or transfection protocols (see, e.g., Sambrook et al. 2003).

In one embodiment, the expressed polypeptide (e.g., BpsA, GS, or ArgZ polypeptide) is an exogenous polypeptide in the host cell, strain, or cell free expression system, which is expressed from a construct according to this disclosure. Alternatively, the polypeptide is expressed from the genome of the host cell or strain. In this embodiment, the polypeptide may be endogenous or exogenous, naturally occurring or non-naturally occurring with respect of the host cell or strain. Preferably the BpsA, GS, or ArgZ so expressed is a functional enzyme as set out herein, or a functional variant thereof. In one specific embodiment, a single host organism or cell free expression system could be utilised to allow expression of multiple polypeptides (e.g., two or more of BpsA, GS, and ArgZ polypeptides, including multiple variant polypeptides), to maximise production.

Host cells and strains useful in this disclosure include E. coli and other strains such as Pseudomonas, Streptomyces, and Bacillus (e.g., Brevibacillus) strains, for example, P. aeruginosa (e.g., P. aeruginosa PAO1), P. syringae (e.g., P. syringae pv. phaseolicola 1448A), P. putida (e.g., P. putida KT2440), P. fluorescens, and B. brevis. Numerous alternative host organisms may be useful in the methods of this disclosure, wherein each cell or strain may provide a different or additional benefit or utility. The choice of an appropriate host strain will affect choice of construct used based on the genetic makeup of the host. Reasons for using different host strains include promoter activity, codon bias, protein solubility, and other factors. Therefore, the use of different host strains provides alternative hosts suitable for use in production of any polypeptide of interest. Cell free expression systems and cell free synthesis systems may also be used in accordance with standard methodology.

In specific embodiments of the present disclosure, the expressed BpsA, GS, or ArgZ polypeptide may be isolated using various biochemical techniques. These techniques include but are not limited to filtration, centrifugation, and various types of chromatography, such as ion-exchange, affinity, hydrophobic interaction, size exclusion, and reverse-phase chromatography. In one particular embodiment, metal affinity chromatography (e.g., nickel or cobalt or others) is used. As exemplifications, the polypeptides may be adsorbed to a solid substrate such as beads, filters, fibres, paper, membranes, chips, and plates such as multiwell plates. The polypeptides may also be prepared as polypeptide conjugates in accordance with known methods.

The BpsA, GS, or ArgZ polypeptides may also be prepared as polypeptide conjugates in accordance with known methods. Protein affinity tags may be used to assist with purification, for example, albumin-binding protein (ABP), biotin-carboxy carrier protein (BCCP), calmodulin binding peptide (CBP), cellulose binding domain (CBP), chitin binding domain (CBD), galactose-binding protein (GBP), glutathione S-transferase (GST), HaloTag®, LacZ, polyhistidine (His-tag), polyphenylalanine (Phe-tag), S-tag, small ubiquitin-like modifier (SUMO), Staphylococcal protein A (Protein A), Staphylococcal protein G (Protein G), Strep-tag, streptavadin, thioredoxin (Trx), tandem affinity purification (TAP), and ubiquitin protein tags. In particular embodiments, the BpsA, GS, and/or ArgZ polypeptide may be produced as a His-tagged polypeptide.

### Biosensor Systems and Uses

Disclosed herein are biosensor systems for rapidly assessing nitrogen levels, including key constituents of yeast assimilable nitrogen (YAN). Advantageously, the biosensor systems and methods do not require specialised laboratory equipment or highly technical expertise. The disclosed systems and methods can be employed without commercial analytics laboratories for nitrogen determination. Previous analytical tests have been costly and relatively inaccessible, resulting in lost time to intervene and limited ability to test samples at multiple time points. The disclosed test involves two or three enzyme catalysed reactions (see, e.g., FIG. 2A), which result in the conversion of organic and inorganic forms of nitrogen to indigoidine, which is a readily visualised blue pigment.

In general embodiments, a biosensor system disclosed herein may comprise a BpsA polypeptide (e.g., SEQ ID NO: 1), a GS polypeptide (e.g., SEQ ID NO: 2 or 13-14), and an ArgZ polypeptide (e.g., SEQ ID NO: 3 or 7-9). Alternatively, a biosensor system may comprise BpsA polypeptide (e.g., SEQ ID NO: 1) and a GS polypeptide (e.g., SEQ ID NO: 2 or 13-14). In specific embodiments, one or more variant polypeptides may be utilised in the biosensor system.

In specific embodiments, the system may include a combination (i.e., co-utilisation) of BpsA, GS, and ArgZ polypeptides or a combination (i.e., co-utilisation) of BpsA and GS polypeptides. In certain aspects, the BpsA, GS, and ArgZ polypeptides (or BpsA and GC polypeptides) may be combined simultaneously, e.g., provided together for the testing format. In other aspects, the BpsA, GS, and ArgZ polypeptides (or BpsA and GC polypeptides) may be combined sequentially. For example, BpsA, GS, and ArgZ polypeptides may be provided one at a time or two at a time to the testing format. As an exemplification, the system components may be combined so as to provide an ArgZ polypeptide first, a GS polypeptide second, and a BpsA polypeptide third. As a further example, the ArgZ and GS polypeptides may be provided first, with the BpsA polypeptide provided after. As a further example, the ArgZ polypeptide may be provided first, with the GS and BpsA polypeptides provided after. As a further example, the GS and BpsA polypeptides may be combined, simultaneously or sequentially.

The biosensor system may be adapted for use with any suitable format. For example, fluid-based formats and solid formats may be utilised. For fluid format assays, various receptacles may be utilised, e.g., tubes, wells, multi-well plates, and bottles. For solid format assays, any number of substrates may be employed. Specific exemplifications include multi-well plates (e.g., microwell plates), point-of-contact components (e.g., beads, conjugates, disks, strips, etc), and point-of-contact means (e.g., dipsticks, chips, columns, slides, arrays, etc). Useful solid substrates include, amongst others, glass, silica, silicon, SU8, polydimethylsiloxane, mica, cellulose, nitrocellulose, paper (e.g., filter paper).

In certain embodiments, the biosensor system may be provided as one or more compositions. The compositions may include one or more of the ArgZ polypeptides, the GS polypeptides, and the BpsA polypeptides as described herein. Noted in particular are solid, semi-solid, and liquid compositions. For example, various powders, tablets, and solution formulations may be utilised. Optionally, the composition may further include one or more buffers, co-factors, cryoprotectors, lyoprotectors, reaction reagents, preservatives, salts, stabilisers, diluents, excipients, or any other components. As a specific exemplification, the biosensor system may be provided in tablet format. For example, any one of the enzymes may be provided as a separate tablet. Two or more enzymes may be combined in a tablet. The tablet may include a freeze dried composition of one or more of the enzymes. The tablet may include one or more binders, stabilisers, excipients, or other components. Useful excipients include, but are not limited to, mannitol, sucrose, microcrystalline cellulose, sodium acetate, isoleucine and poly(ethylene glycol)-4000. The tablet may be formulated to allow dissolving in a suitable fluid, e.g., reaction buffer. An specific exemplification of tabletting is provided in Example 7, herein. Combinations of any of the above biosensor system formats may also be utilised.

In various embodiments, a sample for testing may be obtained and contacted with a testing format that provides a combination of BpsA, GS, and ArgZ polypeptides, or a combination of BpsA and GS polypeptides. In other embodiments, a sample for testing may be obtained and contacted with a testing format that has at least one of the BpsA, GS, and ArgZ polypeptides present, and the remaining polypeptide(s) are provided subsequently. For example, polypeptides (e.g., the GS and BpsA polypeptides) may be pre-combined and provided together, e.g., in a tablet, on a substrate, in a fluid, or any other format. As an alternative to sample removal, the testing device (e.g., strip or dipstick) may be contacted directly with the substance to be tested, for example, insertion into large-scale mixtures, solutions, or materials.

It is envisioned that a sample may be obtained in various forms. For example, the sample may be obtained as a liquid, or may be obtained as a form consisting essentially of liquid, or the sample may be obtained as a semi-liquid, such as a gel, pulp, slurry, suspension, etc, or the sample may be obtained as a solid. When obtaining semi-liquid samples and particularly, solid samples, it may assist in introducing liquid or otherwise liquifying the samples, or extracting the samples, to assist with the testing process.

One or more components of the biosensor system (e.g., one or more of the BpsA, GS, and ArgZ polypeptides) may be dried to facilitate use in particular testing formats. For example, the polypeptides may be freeze dried, i.e., lyophilised. Freeze drying techniques are widely known and commonly used. The freeze drying cycle may be about 48 hours; or ranging from 40 to 56 hours; or 12 to 36 hours; or 36 to 60 hours; or about 40 hours, about 42 hours, about 44 hours, about 46 hours, about 48 hours, about 50 hours, about 52 hours, or about 54 hours. A longer freeze drying cycle, e.g., at least 48 hours (“gentle freeze drying”), may be used to retain maximal activity. In particular embodiments, the process may be carried out to such that water formation is avoided, and the moisture content is minimised during processing.

It may be desirable to use a particular lyophilisation process for obtaining the dried product. For example, a lyophilisation drying program may be used as part of an automated drying system. The lyophilisation process may include multiple drying steps, e.g., with step-wise increases and reductions in temperature. For example, a primary drying setting may be used for sublimation, followed by one or more secondary drying settings that are used to remove residual moisture. In particular embodiments, the top temperature of the lyophilisation process does not exceed 70° C. In other aspects, the temperature of the lyophilisation process ranges between −10° C. to 70° C. In one other aspect, up to 48 hours of lyophilisation is utilised.

Additives may be included with one or more of the polypeptides to assist with freeze-drying. In various aspects, one or more sugar compounds may be utilised as additives. Specific exemplifications include: mannitol, sucrose, trehalose, and any combination thereof. For example, mannitol may be utilised at a range of about 20 mg/mL to about 60 mg/mL, or about 25 mg/mL to about 55 mg/mL, or about 30 mg/mL to about 50 mg/mL, or about 40 mg/mL. Buffers and salts may also be included. For example, Tris buffer may be utilised, such as at a range of about 25 mM to about 75 mM, or about 30 mM to about 60 mM, or at about 45 mM to about 55 mM, or at about 50 mM. For Tris buffers, a neutral or near neutral pH may be utilised, such as a range of about 7 to about 8.5, or about 7.5 to about 8.5, or about 8. As further examples, salts may be utilised, for example, magnesium salts, such as at a range of about 0.5 mM MgCl₂ to about 2 mM MgCl₂, or about 1 mM MgCl₂.

The biosensor system and methods may be adapted for use with any composition to be tested. In various embodiments, the biosensor may be utilised with particular reaction conditions. For example, where fluidic (e.g., microfluidic) testing is utilised, the reaction may include one or more of the following parameters: (1) suitable pH levels, for example, about 6.5 to about 10, or about 7 to about 9, or about 8 to about 9, or about 8.5; (2) suitable buffer levels, for example, achieved with about 80 mM to 120 mM Tris, or about 90 mM to about 110 mM Tris, or about 100 mM Tris; (3) suitable ATP levels, for example, about 8 mM to about 25 mM ATP, or about 10 mM to about 20 mM ATP, or about 15 mM ATP; (4) suitable magnesium levels (Mg⁺²), for example, achieved with about 10 mM to about 30 mM MgCl₂, or about 15 mM to about 25 mM MgCl₂, or about 20 mM MgCl₂; (5) suitable potassium levels (K⁺), for example, achieved with about 5 mM to about 20 mM KCl, or about 8 mM to about 12 mM KCl, or about 10 mM KCl.

The reaction may also include one or more of the following parameters: (1) suitable BpsA polypeptide levels, for example, about 5 μM to about 20 μM, or about 7 μM to about 11 μM, or about 9 μM BpsA; (2) suitable GS polypeptide levels, for example, about 4 μM to about 8 μM, or about 5 μM to about 7 μM, or about 6 μM GS; (3) suitable ArgZ polypeptide levels, for example, about 1 μM to about 15 μM, or about 3 μM to 9 μM, or about 3 μM ArgZ=Other reaction conditions may be utilised to provide optimisation where desired.

The biosensor system may be utilised with various sample sizes. Where fluid based testing is carried out, the ratio of the test solution to the sample solution may be adjusted. In various embodiments, the ratio of the sample solution to the biosensor system solution may be about 5 to about 35 (e.g., about 5:35), or about 4 to about 35 (e.g., about 4:35), or about 3 to about 35 (e.g., about 3:35), or about 2 to about 35 (e.g., about 2:35), or about 1 to about 35 (e.g., about 1:35), or alternatively about 6 to about 35 (e.g., about 6:35), or about 7 to about 35 (e.g., about 7:35), or about 8 to about 35 (e.g., about 8:35), or about 9 to about 35 (e.g., about 9:35), or about 10 to about 35 (e.g., about 10:35), or about 15 to about 35 (e.g., about 15:35), or about 20 to about 35 (e.g., about 20:35), or alternatively, about 1 to about 2 (e.g., about 1:2), or about 1 to about 1 (e.g., about 1:1). Other reaction ratios can be utilised where appropriate.

It is also possible to compare the activities of different combinations of enzymes, and compare the activities of enzymes utilised individually. See, e.g., FIG. 2B. For example, as side by side tests, BpsA can be incubated with a sample on its own (i.e., a one-enzyme biosensor that detects glutamine); BpsA can be incubated with a sample in combination with GS (i.e., a two-enzyme biosensor that detects glutamine and either glutamate or ammonium, depending on which of these two is added exogenously in excess); and BpsA can be incubated with a sample in combination with GS and ArgZ (i.e., a three-enzyme biosensor that detects glutamine, arginine, and ammonium, wherein glutamate is added exogenously in excess).

Amongst other applications, the biosensor system of this disclosure may be utilised for assessments of various samples, including samples comprising: one or more fruits, one or more components of fruit (e.g., skins, flesh, juice, etc), beverage fermentation compositions (e.g., fermenting fruit, fruit components, and/or grains), and food fermentation compositions (e.g., fermenting dairy products, grain products, vegetable products, etc). For performing biosensor assessments, liquid samples may be utilised, or samples consisting essentially of liquid(s). As exemplifications, a sample may comprise fruit juice, a mixture of fruit juices, juice obtained from sampled fruit(s), juice obtained from sampled fruit component(s), or a fermentation liquid (e.g., fermented beverages, finished or in progress).

As noted herein, the biosensor system finds particular use in winemaking and monitoring of grape ripening and/or fermentation. Any grape composition, including grape/grape must fermentation compositions can be tested with the disclosed means and methods. For example, a composition comprising harvested grapes may be used for testing. As another example, grapes ripening on the vine may be used for testing (e.g., pre-harvest grapes). For performing biosensor assessments, liquid samples may be utilised, or samples consisting essentially of liquid(s). As exemplifications, a sample may comprise a grape juice, a mixture of grape juices, juice obtained from the sampled grapes, grape must, juice obtained from sampled grape must, or a fermentation liquid (e.g., wine, finished or in progress).

Exemplary compositions for testing include, for example, compositions comprising Albarino, Airdn, Chenin Blanc, Colombard, Friulano (e.g., Tocai), Gruner Veltliner, Sauvignon Blanc, Traminer, Ugni Blanc, Verdicchio, Verdejo, Vermentino, Viognier, or Zinfandel wine grapes, grape must, and/or grape juice. Other exemplifications include compositions comprising Chardonnay, Gewurztraminer, Gros Manseng, Koshu, Maccabeo, Muscat, Muscadet, Petit Arvine, Petit Manseng, Pinot Blanc, Pinot Gris, Riesling, Scheurebe, Semilion, Sylvaner, or Tokay wine grapes, grape must, and/or grape juice. Still other exemplifications include compositions comprising Cabernet Franc, Cabernet Sauvignon, Carignan, Carmenere, Gamay, Grenache, Malbec, Merlot, Petite Syrah, Petit Verdot, Pinot Noir, Sangiovese, Syrah (also known as Shiraz), or Tempranillo wine grapes, grape must, and/or grape juice. A composition comprising any combination of grapes, grape must, and/or grape juice may be utilised for testing.

For grape growers and winemaking, the biosensor system and methods provide various advantages, including: providing accurate, time effective measurements, generating data for best time to harvest, adding value to crop, avoiding major instrumentation investment and outsourcing costs, allowing centralisation of multiple growers for a single winery, and reducing pressure on time-sensitive decision making. Other advantages include: on the spot testing for harvest; low technical expertise requirement; no specialist instrument requirement; and near instant indication (e.g., via dipstick).

Previously, various types of instruments have been used to measure YAN, including spectrophotometers. Instruments such as the Foss WineScan and Vintessential Chemwell Discrete Analyser have been set up to perform multiple tests important for wine. These instruments are a significant capital investment and would be unobtainable for many small and medium wineries and growers. The Foss WineScan is considered the gold standard of wine testing instruments in New Zealand. Other instruments, such as spectrophotometers also require investment. The revenue model set up by these providers forces users to purchase specific reagents from the company providing the equipment and also uses a service model to continue to generate income from the instruments. The biosensor system and methods of this disclosure avoid these complications and associated costs.

However, it will be recognised that the biosensor system of this disclosure finds many uses beyond winemaking. The disclosed means can be used to measure nitrogen in any sample or source. Therefore, the present biosensor system and methods find significant utility in testing for various research fields and industries, including: beermaking, cidermaking, spirits production, as well as other beverage fermentation activities, and also cheesemaking, yoghurt production, as well as other food fermentation activities. Also encompassed are water and soil testing, e.g., wastewater, agricultural runoff water, freshwater testing, and also pastoral soil, horticultural soil, and forestry soil testing.

Further encompassed is medical testing, e.g., healthcare and diagnostics, for example, relating to multiple sclerosis, cancer, and other health conditions. Accordingly, the biosensor system may be utilised in testing biological samples. As exemplifications, these may include blood samples, urine samples, saliva samples, cerebrospinal fluid samples, lymph fluid samples, eukaryotic cell culture samples, bacterial cell culture samples, amongst others. For example, biological samples can be obtained from a subject and analysed for the levels of nitrogen-containing molecules associated with health conditions or disease states. Assessment of levels of nitrogen-containing molecules can be used to determine the absence, presence, or degree of a health condition or disease state. Levels can be assessed during or following medical treatment to determine whether such treatment has been successful. Biological samples can also include sections of tissues such as frozen sections taken for histological purposes, which can also be analysed for nitrogen-containing molecules. Other samples, including biological samples, can be those derived from cell culturing and amino acid production. If the sample is initially complex, solid, or viscous, it can optionally be treated, such as by extraction, or it can be dissolved or diluted in order to obtain a sample having the appropriate characteristics for use in the disclosed assays. Non-biological samples may also be utilised for testing, e.g., a chemically-synthesised composition.

Therefore, the biosensor system and methods are not limited to testing any particular composition or type of composition.

The examples provided herein are presented for the purpose of illustrating specific embodiments and aspects and are not intended to limit the present disclosure in any way. Persons of ordinary skill can utilise the findings and teachings herein to produce other embodiments, aspects, and variations without undue experimentation. All such embodiments, aspects, and variations are considered to be part of this disclosure.

### EXAMPLES

### Overview of Experiments

Yeast Assimilable Nitrogen (YAN) is an important component in winemaking. It primarily consists of ammonium and free amino acids, in particular glutamine and arginine. Not enough YAN during fermentation can result in stuck fermentation where the fermentation process stops even though there are sufficient sugars to continue. It can also result in the production of molecules with undesirable taste characteristics, such as hydrogen sulphide (AWRI, 2016). The current testing systems for YAN are too complicated and expensive for most winemakers to use on-site, instead requiring samples to be sent to commercial laboratories, which is costly and time-consuming.

Our goal has been to develop an enzyme-based biosensor that provides an easy to use, robust, and inexpensive on-site test for YAN. In previous work, the blue pigment synthetase A (BpsA) enzyme was developed and patented to quantify glutamine in human biological samples by converting it into a blue dye, indigoidine. We have found that BpsA can also quantify glutamine in grapes (e.g., grape juices, grape musts, wines, etc), giving a way to quantify one of the main constituents of YAN.

Now, as demonstrated herein, we have coupled BpsA with two other enzymes: arginine dihydrolase and glutamine synthetase, which sense and report on ammonium and arginine: two other major constituents of YAN. A variety of arginine dihydrolase and glutamine synthetase candidates were expressed, purified and characterised. One key consideration was the ability of an arginine dihydrolase, a glutamine synthetase and BpsA to function in a one-pot reaction with untreated grape juice samples. Optimisation yielded a three-enzyme cascade that reports on YAN at the level of accuracy required by winemakers. Additionally, we have shown that all three enzymes can be lyophilised for improved storage stability in a commercial setting.

### Aims of Experiments

The first aim of these studies has been to identify and characterise a suitable ammonium sensing enzyme, glutamine synthetase. We looked for an enzyme to be active, stable, and lack product inhibition. We looked also for activity under conditions used for BpsA.

The second aim of these studies has been to identify and characterise a suitable arginine sensing enzyme. For this study, arginine deiminase and arginine dihydrolase have been considered. We looked for an enzyme to be active and stable. We looked also for activity under conditions used for GS-BpsA.

The third aim of these studies has been to assemble and optimise the three-enzyme cascade to compare with the commercially available tests.

The fourth aim of these studies has been to assess performance parameters, including detection ranges, and the performance of different enzymes in the biosensor system.

### Example 1: Identification of Glutamine Synthetase Candidates

For preliminary experiments, glutamine synthetase from Escherichia coli (E. coli) was successfully expressed, purified and tested for activity. This assay took advantage of the biosynthetic reaction of GS (which produces ADP) coupled with lactate dehydrogenase and pyruvate kinase to oxidise NADH (Shapiro & Stadtman, 1970).

The reaction mixture included 30 mM imidazole, 100 mM Na-glutamate, 8.5 mM adenosine 5′-triphosphate (ATP), 1 mM phosphoenolpyruvate (PEP), 60 mM magnesium chloride (MgCl₂), 20 mM potassium chloride (KCl), 45 mM ammonium chloride (NH₄Cl), 0.25 mM 0-nicotinamide adenine dinucleotide (NADH), 28 units pyruvate kinase/40 units L-lactic dehydrogenase (PK/LDH). The reactions were performed at 37° C. and rates were measured by the decrease of absorbance at 340 nm.

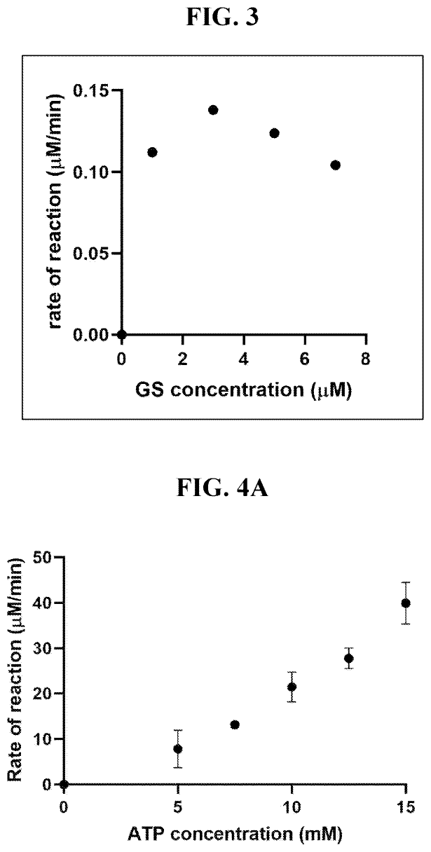

The GS from E. coli was only slightly active and did not show the expected linear increase in activity with increasing enzyme concentration (FIG. 3). These preliminary experiments were useful for establishing the GS activity assay. Yet, the results suggested that the E. coli enzyme was not a good choice for a YAN biosensor. In addition to its low activity in vitro, it is known that the E. coli GS is negatively regulated by adenylation (Stadtman, 2004). It is likely that in vivo adenylation results in a heterogeneous (and largely inactive) preparation of enzyme.

### Datamining and Preliminary Testing

More suitable GS candidates were sourced by mining the enzyme database, BRENDA (https://www.brenda-enzymes.org; see, e.g., Chang et al. (2021) Nucleic Acids Research, 49, D498-D508). We looked for GS enzymes with appropriate characteristics for the biosensor, e.g. structural, functional and regulatory properties.

Five alternative GS enzymes were identified as worthy of further study (Table 1).

TABLE 1
GS candidates and their traits

| Source of GS | Traits |
|---|---|
| Corynebacterium | Industrial producer of glutamine (Harper et |
| glutamicum | al., 2008) |
| Helicobacter pylori | Lacks adenylylation (Joo et al., 2018) |
| Leishmania donovani | Has been expressed and purified in E. coli |
| | (Kumar et al., 2017) |
| Pseudomonas putida | Has not been extensively studied, which added |
| | to the novelty of the biosensor |
| Trypanosoma cruzi | Has been expressed and purified in E. coli, |
| | optimum pH of 8 is the same as BpsA (Bender |
| | et al., 1977) |

The polynucleotide sequence of each of the GS candidates was codon-optimised for expressing the corresponding polypeptide in E. coli and the codon-optimised DNA sequence, inserted into pET28a, was ordered from Twist Bioscience. Each pET28a-GS plasmid was used to transform the E. coli E. cloni 10G cloning strain and the E. coli BL21(DE3) Gold expression strain. Each GS candidate was screened for soluble protein expression using a number of expression conditions. Optimised expression conditions are summarised in Table 2, below. Among the five candidates, three were expressed as soluble proteins: C. glutamicum GS, H. pylori GS, and T. cruzi GS. L. donovani GS, and P. putida GS could not be expressed in a soluble form.

TABLE 2
GS candidates and their expression conditions

| | | Approximate yield |
|---|---|---|
| | | (mg purified protein/ |
| Candidates | Expression condition | L culture) |
| C. glutamicum | Autoinduction media, | 40 |
| | 30° C., 16 hours | |
| H. pylori | Lysogeny broth, IPTG, | 71 |
| | 30° C., 4 hours | |
| L. donovani | — | — |
| P. putida | — | — |
| T. cruzi | Lysogeny broth supplemented | 14 |
| | with 100 mM ammonium and | |
| | glutamate, IPTG, 16° C., | |
| | 24 hours | |

The soluble GS enzymes were tested. C. glutamicum GS, H. pylori GS, and T. cruzi GS, were coupled with PK/LDH and BpsA to test their activity. The BpsA test was carried out by a two-step assay. GS assays were carried out first using the reaction conditions (as described in FIG. 3) for 20 minutes. Then, an aliquot of the first assay was added into the BpsA reaction mixture. C. glutamicum GS and H. pylori GS were both active and glutamine production was detected with BpsA, however, no activity was detected when the PK/LDH assay was used to detect ATP hydrolysis. On the other hand, the T. cruzi GS showed ATP hydrolysis activity, but no glutamine was detected via BpsA.

The design of the YAN biosensor asks for GS to produce glutamine under conditions that are compatible with BpsA. For this reason, T. cruzi GS was discarded from the pipeline. The two remaining candidates were tested in a one-pot reaction with BpsA, under optimal reaction conditions for the latter (50 mM Tris-HCl pH 8.5, 20 mM MgCl₂, 6 mM ATP).

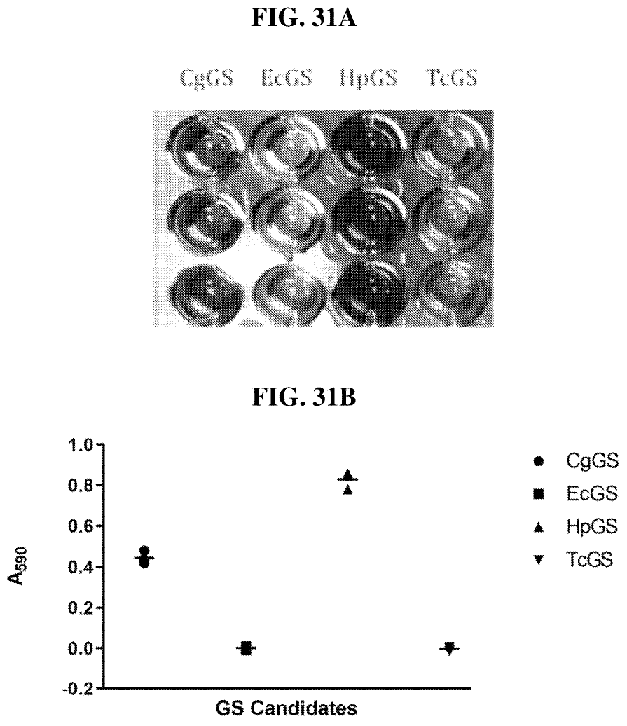

CgGS showed activity in the one-pot reaction. However, it was ranked as the second-best candidate for the biosensor. The reasoning for this is noted as follows. Overnight incubation (˜16 hours) was required to obtain high levels of recombinantly expressed CgGS. In contrast to this, 4 hours of expression was sufficient for HpGS, and higher levels of expression were achieved with HpGS (Table 2). CgGS was also less active than HpGS in optimal assay conditions where the activity read-out was via coupling with BpsA (FIG. 31). Finally, it was noted that CgGS could be post-translationally inactivated by adenylation (Beckers et. al., 2001), which could complicate recombinant production of the enzyme.

Therefore, the H. pylori GS (HpGS) was considered the top-ranked enzyme in these tests. The reasoning for this is noted as follows. It was not subjected to post-translationally modification by adenylation (Joo et al., 2018): It was overexpressed and purified with high yield (Table 2). It was also found to be the most active GS enzyme in the same assay conditions as BpsA. Nevertheless, our findings indicate that CgGS may also be utilised in biosensor systems in accordance with this disclosure.

### Example 2: Experimental Analysis of Selected Glutamine Synthetase

### Initial Optimisation

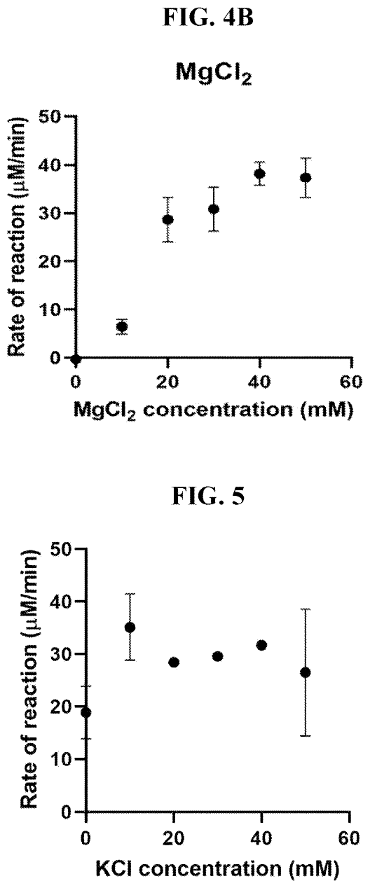

Both GS and BpsA require a high amount of ATP for optimal activity (Shapiro & Stadtman, 1970; Brown et al., 2017). ATP can affect the pH of buffers, therefore the pH buffering for the assay was set for investigation. To start, an experiment was carried out with increasing concentrations of ATP to determine the minimum concentration of ATP required for activity. An experiment was also carried out to determine the effect of Mg²⁺ on activity by testing the activity of GS-BspA in increasing concentrations of MgCl₂. FIG. 4 shows the effect of ATP concentration and MgCl₂ concentration on activity.

For the ATP concentration trial, the buffer used in the assay was 100 mM Tris pH 11.4. An excess of MgCl₂, KCl, glutamate, NH₄Cl, was in the reaction mix. For the MgCl₂ concentration trial, the reaction was in 100 mM Tris pH 11.4 with 15 mM ATP, and an excess amount of KCl, NH₄Cl, and glutamate. GS concentration was 3 μM while BpsA concentration was 6 μM in both assays. The assay was carried out at 37° C. The rate was measured by the increase in the absorbance at 590 nm for 9 minutes.

It was observed that, as the ATP concentration increases, the pH of the buffer solution decreases. Different combinations of ATP and buffers were tested. The best performing combination is shown in FIG. 4A. The pH of 100 mM Tris-base is 11.4. It was noted that ATP is acidic once hydrolysed (Åström et. al., 2004); thus it serves a dual purpose in the assay.

First, it lowers the pH to ˜9, which is in the buffering range for Tris. Second, it serves as a substrate for both HpGS and BpsA in the assay. A concentration of 15 mM was chosen for future experiments since HpGS and BpsA are most active at that concentration and the ATP is able to lower the pH so that Tris acts as a suitable buffer. Higher ATP concentrations were also tested; however, there appeared to be a problem with precipitation under these particular conditions.

Next, MgCl₂ concentration was varied under optimal ATP/pH conditions (FIG. 4B). Both HpGS and BpsA use Mg²⁺ as a co-factor so it was important to ensure this was not limiting the enzyme activity. A concentration of 20 mM MgCl₂ was chosen for future experiments since there were maximal rates of enzyme activity at this concentration.

Lastly, the amount of KCl added to the assay was optimised. KCl was added to assist with GS stability during the assay; however, in excess it could lead to destabilisation. FIG. 5 shows optimisation of KCl concentration in the assay. For the KCl concentration trial, the reaction consists of 100 mM Tris Base pH 11.4, 15 mM ATP, 20 mM MgCl₂, and an excess amount of NH₄Cl and Na-Glu. GS concentration was 3 μM while BpsA concentration was 6 μM in the assay. The assay was carried out at 37° C. The rate was measured by the increase in the absorbance at 590 nm for 9 minutes.

Optimised ATP/pH and Mg²⁺ conditions were utilised with varying concentrations of KCl (FIG. 5). A concentration of 10 mM KCl was chosen to be optimal based on this experiment. The final optimised reaction mixture for the GS-BpsA assay was determined to be 100 mM Tris Base pH 11.4, 15 mM ATP, 20 mM MgCl₂, and 10 mM KCl.

### Kinetic Analysis of Glutamate and Ammonium

Using optimised assay conditions for the GS-BpsA coupled assay, kinetics data for HpGS were collected for its substrates glutamate and ammonium. For these experiments, the concentrations of GS and BpsA were 3 μM and 6 μM respectively. Optimised conditions for the GS-BpsA assay were used. A range of 0-1000 μM glutamate concentration and 1000 μM NH₄Cl was used in the assay. Rates were measured through the increase in absorbance for 5 minutes at 590 nm and 37° C.

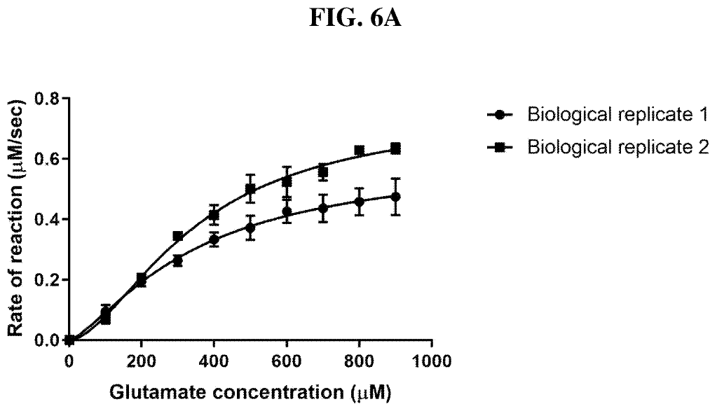

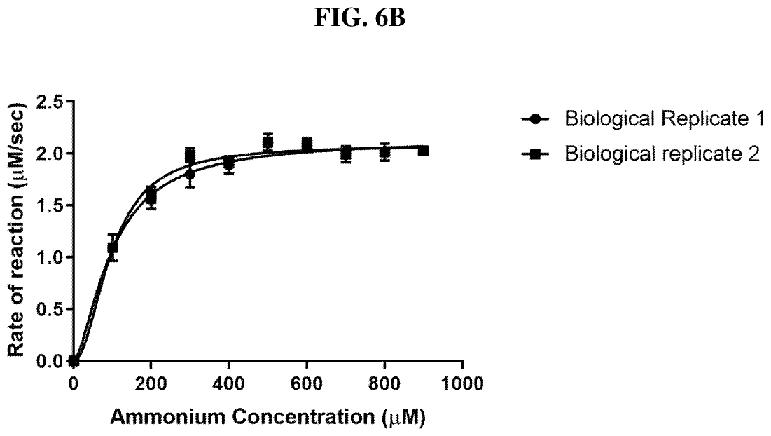

GS enzymes have been previously found to display non-Michaelis-Menten, sigmoidal kinetics (Murray et al., 2013). As expected, the data were fit better by the Hill equation for cooperative enzymes than by the Michaelis-Menten equation (FIGS. 6A-6B). The Hill coefficient indicates the estimate of the number of active sites that are acting cooperatively, which has a value of approximately 2 for both substrates. See Table 3, below.

TABLE 3
Summary of the kinetic parameters of
HpGS across two biological replicates

| | |
|---|---|
| Glutamate | Ammonium |

| | K_{half} | k_{cat} | K_{half} | k_{cat} |
|---|---|---|---|---|
| | (μM) | (s⁻¹) | (μM) | (s⁻¹) |
| Biological Replicate 1 | 350 ± | 0.28 ± | 96 ± | 0.78 ± |
| | 70 | 0.02 | 7 | 0.01 |
| Biological Replicate 2 | 350 ± | 0.45 ± | 97 ± | 0.78 ± |
| | 30 | 0.04 | 5 | 0.02 |

| | | |
|---|---|---|
| Hill coefficient | 1.5 | 2.0 |

The Khalf, which is the concentration of the substrate at the half-maximal velocity of HpGS was 350 μM for glutamate and approximately 100 μM for ammonium. Moreover, the Vₘax observed when glutamate was varied was lower than when ammonium was varied. The Vₘax values were used to estimate the k_{cat} parameters, which were calculated to be 0.3±0.01 s⁻¹ and 0.8±0.03 s⁻¹ for glutamate and ammonium, respectively.

### Compatibility of HpGS and BpsA

Assays were carried out for the detection of glutamate and ammonium in grape juice and wine. HpGS and BpsA were tested for their ability to detect glutamate, ammonium, and glutamine in grape juice samples. A 1:2 ratio of GS:BpsA was used in each assay (3 μM and 6 μM respectively) to ensure that BpsA did not limit the reaction.

The first trial used grape juice including pressed Pinot Gris (PP), white table grape juice muslin filtered (WGF), white table grape juice charcoaled (WGC), red table grape juice charcoaled (RGC). For each sample, a 100% concentrate or a 50% dilution were tested. GS was used to quantify either glutamate or ammonium, while BpsA was used to quantify glutamine in the sample.

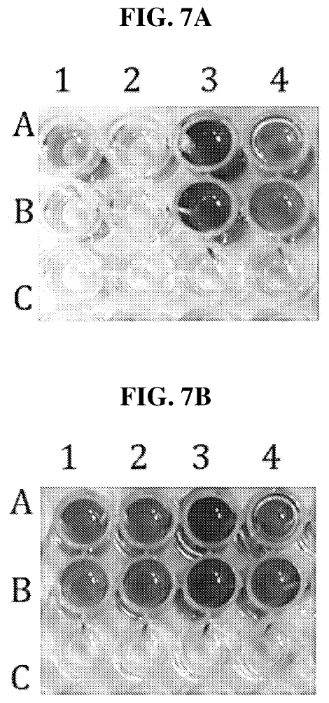

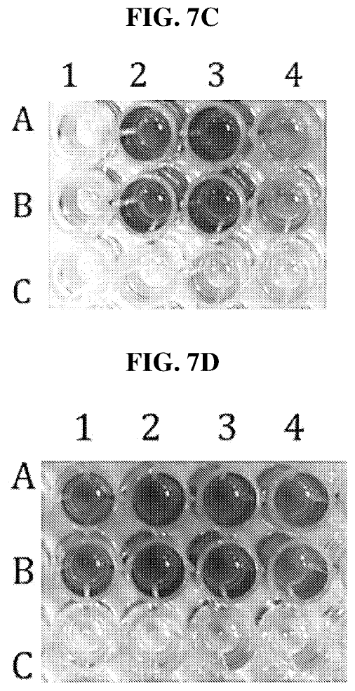

Ammonium is known to be abundant in grape samples (Nicolini et al., 2004) thus, 500 μM glutamate was also added to the assay to supplement the lack of glutamate for ammonium quantification. Analysis included GS-BpsA tests (detecting ammonium+glutamate+glutamine), and BpsA tests (detecting glutamine only). Four samples were assessed: 100% grape sample, 100% grape sample with 500 μM glutamate, 50% grape sample, and 50% grape sample with 500 μM glutamate. The results are set out in FIGS. 7A-7D.

These results show that GS-BpsA can detect ammonium, glutamate, and glutamine in grape juice. FIGS. 7A and 7C correspond to the test without the addition of glutamate. The indigodine produced in this test shows the amount of glutamate+glutamine in the sample. FIGS. 7B and 7D corresponds to the test with the addition of 500 μM glutamate. The amount of indigoidine produced in the test indicates the amount of ammonium+glutamine in the sample. Row C in every figure corresponds to the BpsA test detecting only glutamine. The difference in indigoidine produced in each test can indicate the amount of ammonium, glutamate, and glutamine in the sample. Moreover, the overall intensity of the indigoidine produced corresponds to the total amount of the three YAN constituents.

By using the intensity of absorbance of indigoidine at 590 nm, we can quantify either the total or the separate amount of ammonium, glutamate, and glutamine in the sample. Moreover, without using a spectrophotometer it can be inferred that in grape juice there is less glutamate and glutamine while there is a high level of ammonium. Since there is an excess of ammonium, the addition of glutamate is used to quantify the total amount of ammonium in the sample.

For the second trial, only wine grape samples (Pinot Noir and Pinot Gris grape juices) from different pre-treatment stages were tested. Testing the wine grape samples from unprocessed and different pre-treatment stages is important to determine the ease of use of the test for winemakers.

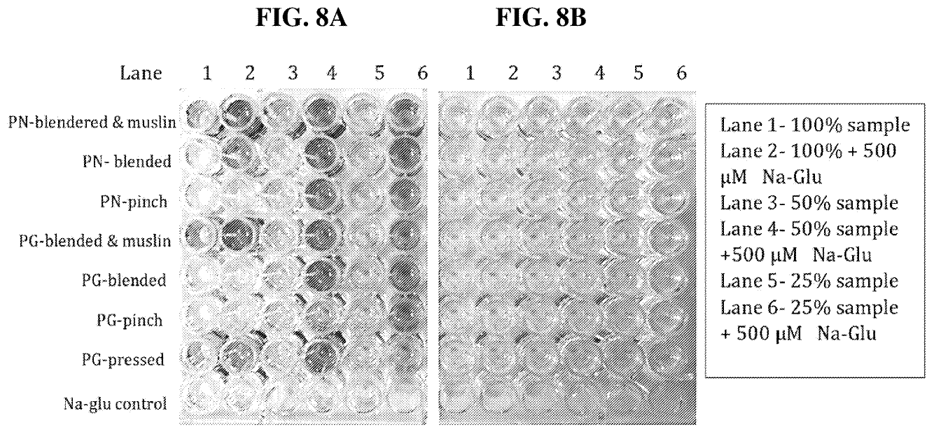

For these experiments, GS-BpsA was utilised with wine grape samples. Different concentrations of wine grape samples were used. Ammonium and glutamate concentration can be inferred from the difference of indigoidine produced with the sample only and the sample with excess glutamate added. A glutamate control was included to ensure that the added glutamate in the samples is pure and no glutamine contaminant was being introduced. BpsA with grape wine samples was used to show the amount of glutamine present in the grape wine samples. FIG. 8 presents the results of this trial.

The results demonstrate that the GS-BpsA test works even in the unprocessed grape wine samples. The GS-BpsA assay reports the amount of ammonium and Na-Glu even with dilution, which lowers the acidity and possible interference of other molecules present in the sample. The difference in the indigoidine produced between the sample with and without the addition of glutamate indicates the amount of ammonium and glutamate respectively once the glutamine is subtracted (FIG. 8A). The BpsA test was used to quantify glutamine (FIG. 8B). No indigoidine was produced in the BpsA test (FIG. 8B). This indicates that the indigoidine produced in the GS-BpsA test was from the GS converting glutamate and ammonium into glutamine.

### Example 3: Further Optimisation of GS-BpsA Test for Wine Grape Samples pH, ATP, and Volume Optima

Further optimisation was proposed for the GS-BpsA test. The pH of wine grape samples was measured to be around pH 3.5, which can alter the overall pH of the reaction. With this, the pH of the test reaction mix and the ratio of sample volume and test reaction volume were varied and assessed. As indicated above, ATP is responsible for lowering the pH of 100 mM Tris pH 11.4 indicating that it can act as an effective buffer. For this reason, ATP concentration was further optimised.

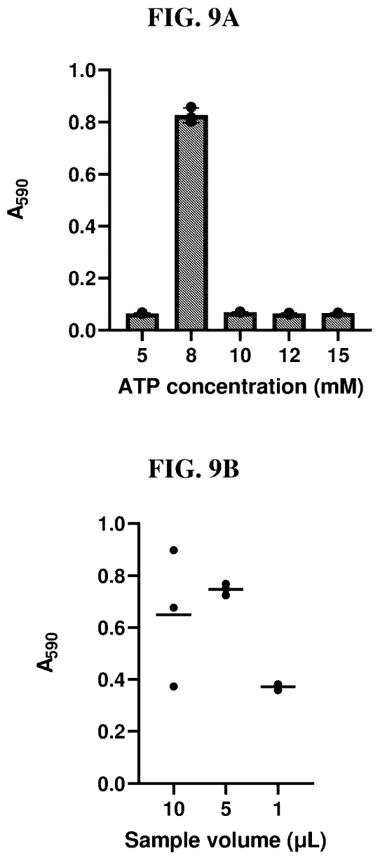

For ATP optimisation, a varying concentration of ATP from 5 mM to 15 mM in the reaction was tested. For sample volume optimisation, a sample volume of 1 μL, 5 μL, and 10 μL was used in the assay. The reaction mixture was added to make the total volume up to 40 μL with the same final concentration of each of the components. The sample used was Pinot Gris that had been blended and muslin filtered. Ten millimolar glutamate was added to ensure the complete conversion of ammonium to glutamine. The reaction was incubated for 1 hour before the resolubilisation of indigoidine with DMSO. FIG. 9 sets out the optimisation results.

As described, a varying concentration of ATP was added in the test reaction with wine grape sample until the desired pH was achieved. An optimised ATP concentration was determined to be 8 mM. (FIG. 9A).

Using the newly optimised reaction condition, the ratio between sample and test reaction mix was then tested. A BpsA test for L-glutamine in biological samples has been previously suggested (Brown et al., 2017). The standard reaction described in that work uses 10 μL of sample and 30 μL of reaction mix totaling 40 μL and is then incubated for 1 hour while shaking at 200 rpm. Resolubilisation of indigoidine was made by adding DMSO to a total concentration of 83% v/v then incubated for 20 minutes while shaking at 2000 rpm. Following this as the standard protocol, a sample: test reaction ratio of 10:30, 5:35, and 1:39 was tested. The ratio of 5:35 was determined in these particular assays as useful for reducing interference (FIG. 9B).

### Incubation Time and GS-BpsA Optima

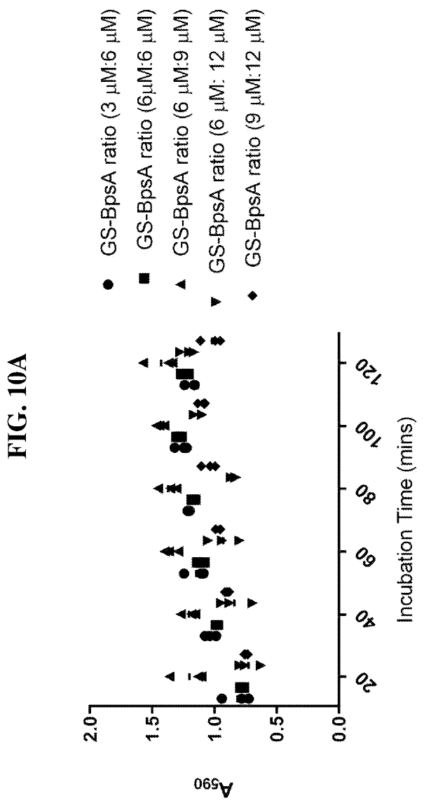

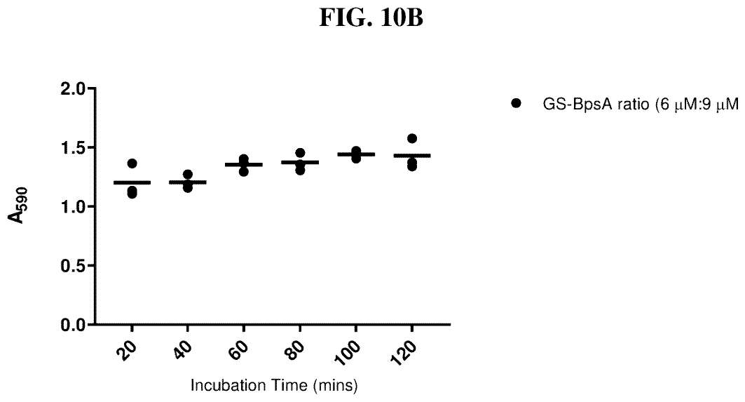

As noted previously, a GS concentration of 3 μM and a BpsA concentration of 6 μM were used for the GS kinetics and the compatibility test. These concentrations were enough for a complete conversion of pure ammonium (from NH₄Cl) and pure glutamate (as Na-Glu) into glutamine, then indigoidine, in an hour. However, there are a variety of biomolecules in grape and other biological samples that might interfere or affect the activity of both GS and BpsA. Thus, it was important to verify that the assay had gone to completion. For this, different GS-BpsA ratios in the assay and incubation times were tested (FIG. 10).

For incubation time optimisation and GS-BpsA concentration optimisation, a reaction mix with different GS-BpsA ratios was prepared and used for the test. The sample used for this test was Pinot Gris that was blended and muslin filtered. Ten millimolar Na-Glu was added for the complete conversion of ammonium. The reaction was stopped in 20 minutes increments and indigoidine was resolubilised using DMSO, before taking absorbance readings at 590 nm.

Among various combinations of GS-BpsA concentration, a notable increase in signal was seen from the GS-BpsA concentration ratio of 6 μM and 9 μM respectively (FIG. 10A). Then, the signal produced by GS-BpsA (6 μM: 9 μM) was compared through time. As shown in FIG. 10B, only a slight increase in the signal was observed from 60 minutes to 120 minutes incubation. This indicated that a 60 minute incubation was sufficient.

Overall, an optimised reaction mixture for GS-BpsA testing included 0.1 M Tris pH 11.4, 8 mM ATP, 10 mM KCl, 20 mM MgCl₂, 6 μM HpGS, 9 μM BpsA. An optimised sample-reaction ratio was 5:35 and the selected incubation time was 60 minutes.

### Ammonium Concentration in Wine Grape Samples Throughout Fermentation

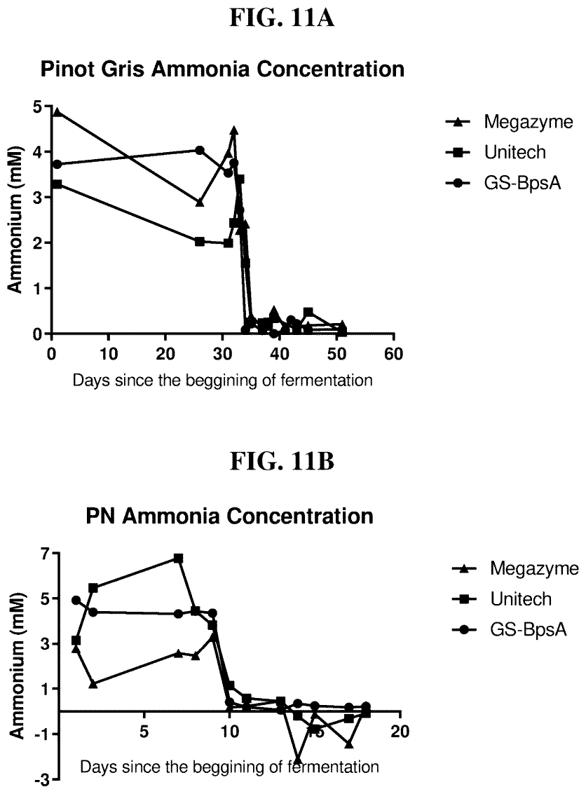

An optimised GS-BpsA test was used to quantify ammonium in wine grape samples throughout fermentation (FIG. 11). Commercial test kits for ammonium (Unitech and Megazyme) were used for data validation. The samples used were from Mt. Difficulty Wines, and no pre-treatment was made before testing. Ammonium quantification was carried out for Pinot Gris and Pinot Noir. The testing was done side by side using GS-BpsA, Unitech, and Megazyme, following each protocol.

The GS-BpsA test results, if not exact, were comparable with the two other commercial test kits (FIGS. 11A-11B). Surprisingly, the results gathered from Unitech and Megazyme were different during the early days of fermentation.

It was concluded that the H. pylori glutamine synthetase is the suitable candidate for the ammonium-sensing enzyme to be paired with BpsA. In comparison to the other GS candidates, it lacks the capacity to be modified post-translationally (adenylylation). This resulted in an active, well-expressed, and purified enzyme. Additionally, HpGS passed the compatibility trial with BpsA and its reaction conditions. More importantly, the GS-BpsA coupled enzyme test can be optimised and successfully measure ammonium and glutamine in wine grape samples. This was verified by comparing GS-BpsA coupled test with two of the current ammonium test kits available in the market.

### Example 4: Identification of Candidate Arginine Metabolising Enzymes

Initial experiments focused on arginine deiminase enzymes (ADIs) and are summarised briefly here to emphasise that the initial choice was not the correct one. Instead, the most appropriate enzymes were determined to be arginine dihydrolases (ArgZs).

A survey of the literature identified three initial ADI candidates, from Pseudomonas putida, Mycoplasma hominis, and Pseudomonas aeruginosa. The P. putida and M. hominis enzymes were chosen because of their history of being used as biosensors (Verma et al., 2015; Zhybak et al., 2017). On the other hand, the P. aeruginosa enzyme was selected because it has been recombinantly expressed and purified in Escherichia coli as a soluble and active enzyme (Galkin et al., 2004).

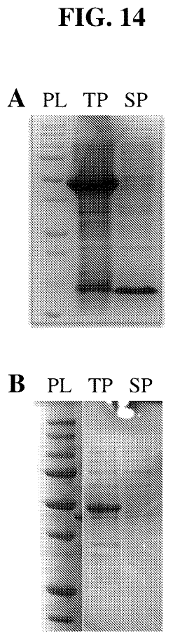

We attempted to express and purify the three ADI enzymes in E. coli. After numerous attempts, we were unable to express the M. hominis or P. putida enzymes in a soluble form. Each protein was expressed solely in inclusion bodies, under all conditions tested (FIG. 14A). Purification from inclusion bodies and refolding were also attempted, without success. In contrast, we were able to express and purify the P. aeruginosa ADI enzyme (average yield=40 mg per litre of bacterial culture). However, when the purified enzyme was assayed, it did not have any activity.

At this time, we learned of the newly-discovered enzyme, ArgZ. In the study conducted by Zhuang et al. (2020), the ArgZ protein from Synechocystis sp. PCC6803 was shown to have three domains: an N-terminal domain for the guanidino-group modifying enzyme (comprising amino acid residues 1-269), a middle domain from residues 286-356 which is analogous to the N-terminal conserved domain of the lysine-oxoglutarate reductase/saccharopine dehydrogenase bifunctional enzyme, and an uncharacterised C-terminal domain (residues 364-705). The N-terminal domain of ArgZ (ArgZ-N) has the arginine dihydrolase activity, and is sufficient to convert arginine to ornithine and ammonium (Zhang et al., 2018).

For this reason, we ordered the gene for this isolated domain from a commercial gene synthesis company (Twist Bioscience). In parallel, we ordered the genes for three other ArgZ enzymes, from Methylosinus trichosporium, Streptomyces coelicolor, and Rhodospirillum centenum. The Rhodospirillum centenum ArgZ sequence used included deletion of amino acid residues 2 to 18 from the UniProt sequence B6IRA8. These residues were deleted because they were predicted to encode a disordered region in the polypeptide. Accordingly, it will be understood that the RcArgZ polypeptide and polynucleotide sequences (e.g., SEQ ID NO: 3 and 6) as disclosed herein are non-naturally occurring sequences.

The ArgZ enzymes from these organisms were previously shown to have the desired arginine dihydrolase activity in “yes/no” tests (Zhuang et al., 2020), however, no in-depth studies were conducted. Details on the enzymes are provided in Table 4. In our hands, the well-characterised Synechocystis ArgZ-N had the poorest yields when expressed and purified from E. coli.

TABLE 4
Arginine dihydrolase candidates and their
yields upon expression in E. coli

| | GenBank | | |
|---|---|---|---|
| | accession | Approximate yield (mg purified | |
| Enzyme | number | protein per L of culture) | |
| Synechocystis sp. | BAA18641.1 | 5 | mg |
| ArgZ-N (truncation) | | | |
| M. trichosporium ArgZ | ATQ6954.1 | 7 | mg |
| S. coelicolor ArgZ | NP625511.1 | 80 | mg |
| R. centenum ArgZ | ACI97994.1 | 100 | mg |
| (truncation) | | | |

The activities of all four enzymes were tested in two different assays, and all four were active in both tests. Optimised GS/BpsA reaction conditions were used for the testing (see above). Each ArgZ was present at 10 μM. The only source of nitrogen for indigoidine production was arginine (0.6 mM). Glutamate was present at 2 mM, so that the ammonium liberated from arginine could be incorporated into glutamine, and then indigoidine, by GS and BpsA respectively.

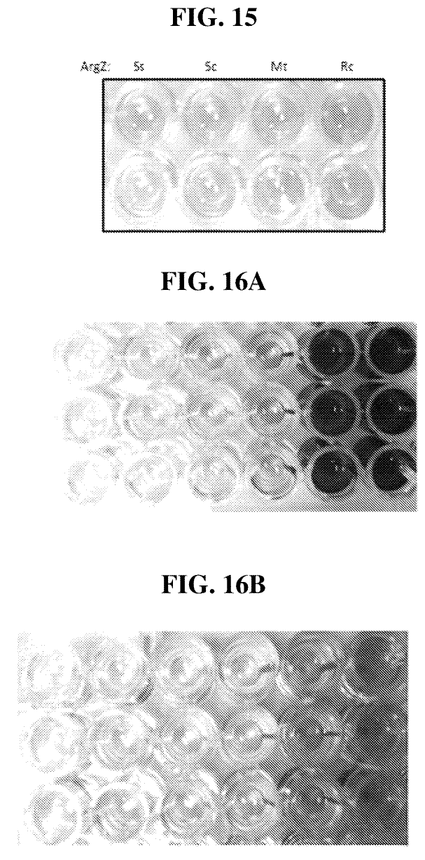

Of most relevance for our biosensor, the ArgZ enzymes were each mixed with GS and BpsA in a single tube, under the conditions (buffer, pH, cofactors, etc) that had previously been optimised for GS and BpsA alone. As shown in FIG. 15, all four ArgZ enzymes could liberate ammonium from arginine, which in turn could be detected via its conversion to indigoidine by GS and BpsA. This result showed that all four ArgZ enzymes could find utility in the disclosed biosensor.

With all four candidates showing activity in vitro, we considered other factors that might affect their application in accordance with the disclosed biosensor systems. M. trichosporium ArgZ and Synechocystis ArgZ-N were recombinantly produced with comparatively low yields (Table 4). The other two enzymes were re-tested in the GS/BpsA coupled assay across a range of arginine concentrations (FIGS. 16A-16B).

To test range and activity, R. centenum ArgZ was coupled with GS and BpsA, and S. coelicolor ArgZ was coupled with GS and BpsA. An optimised GS/BpsA reaction condition was used for the assay (see above) with increasing concentrations of arginine. An excess of glutamate was added to ensure the complete conversion of the ammonium produced from arginine, into indigoidine.

The results showed that R. centenum ArgZ (RcArgZ) was the most active in the GS/BpsA coupled assay as seen with the intense indigoidine production (FIG. 16A). Under identical conditions, the S. coelicolor ArgZ (ScArgZ) enzyme also produced indigoidine, although we observed that these levels were lower than those produced by RcArgZ (FIG. 16B). Therefore, RcArgZ was found to be the top-ranked enzyme in these tests. Yet, it is clear from our results that ScArgZ may also be utilised in biosensor systems in accordance with this disclosure.

### Example 5: Experimental Analysis of Selected Arginine Dihydrolase Enzyme

RcArgZ activity was quantified in vitro by coupling it with glutamate dehydrogenase (GDH) in a spectrophotometric assay. GDH converts α-ketoglutarate and ammonium to glutamate via oxidation of NADH to NAD⁺ (Plaitakis, Kalef-Ezra, Kotzamani, Zaganas, & Spanaki, 2017).

NADH absorbs light at 340 nm while NAD⁺ does not. Therefore, a decrease in absorbance at 340 nm is directly relative to glutamate production, and because GDH is in excess in the reaction, it is directly relative to RcArgZ activity to produce ammonium from arginine.

### pH Optima

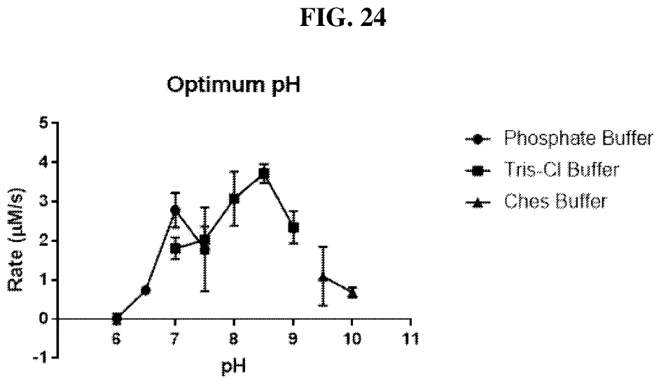

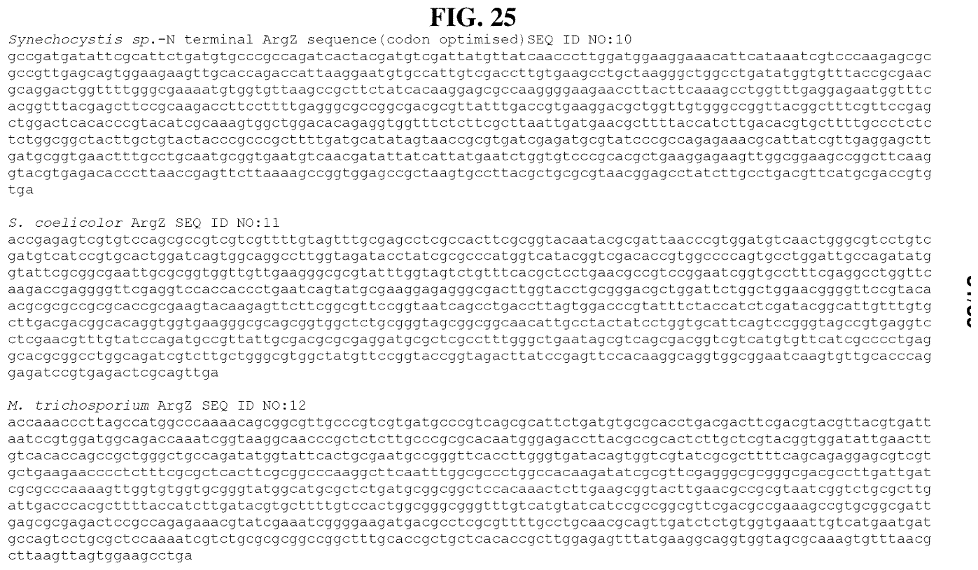

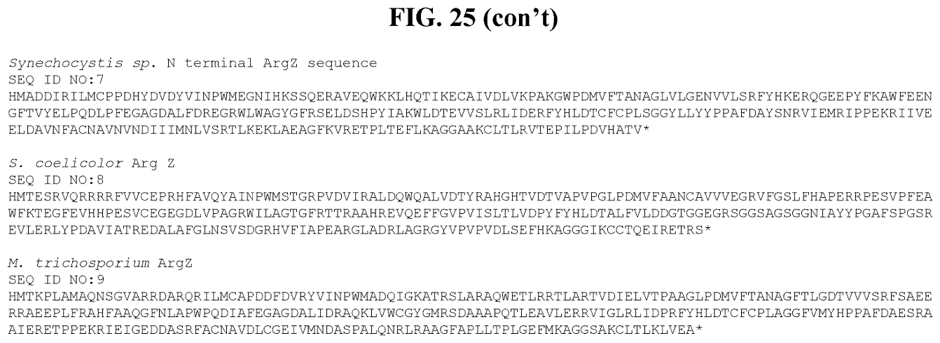

The activity of RcArgZ over a range of pH values from 6.0 to 10.0 was measured. Activity was observed from pH 6.5 to pH 10, with an optimum pH of 8.5 (FIG. 24). Conveniently, this matched optimum pH levels for the GS/BpsA coupled assay.

For these experiments, 100 millimolar phosphate, Tris-Cl, and CHES buffers were used to buffer assays in the assay at different pH levels. Each reaction consisted of the buffer, 5 mM NADH, 10 mM arginine, 10 mM α-ketoglutarate, and 70 U of glutamate dehydrogenase. The assay was started upon the addition of 1 μM RcArgZ at 25° C. The rate was measured by the decrease in absorbance at 340 nm for 5 minutes. Data are the mean values of three replicates and error bars indicate the standard error of the mean (FIG. 24).

### Kinetic Testing

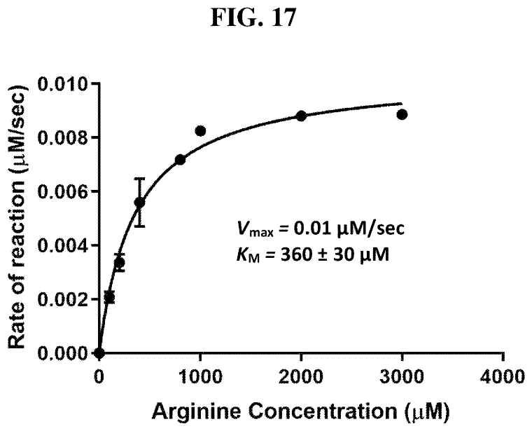

Activity data for RcArgZ were collected across a range of arginine concentrations. Measurements at each substrate concentration were collected in technical triplicate and the Michaelis-Menten equation was fitted to the data (FIG. 17). For this experiment, a range of 0-3000 μM arginine concentration was used in the assay. Rates were measured through the decrease in absorbance at 340 nm at 37° C. The results showed that RcArgZ has a Michaelis constant (KM) of 360±30 μM and a k_{cat} of 10.4 s⁻¹.

### Example 6: Further Testing for ArgZ-GS-BpsA Biosensor System

### Freeze Drying Procedures

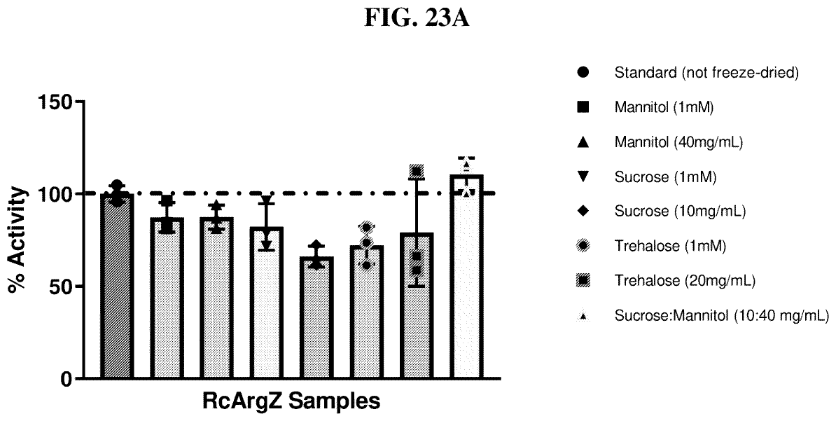

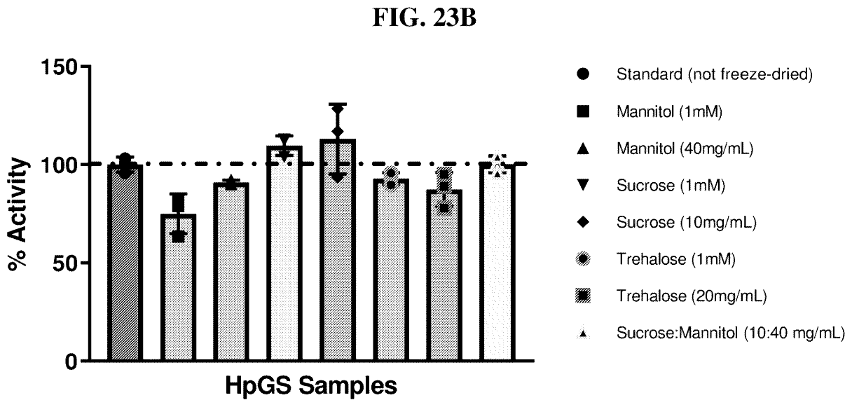

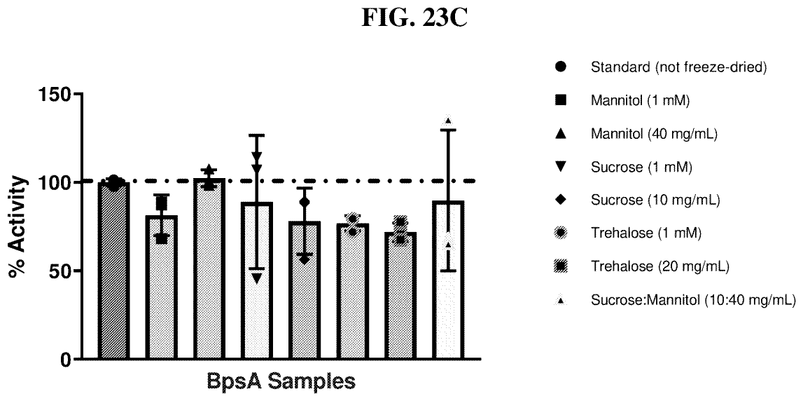

Freeze-drying can help with the long-term stability of the enzymes, however, not all enzymes are amenable to this treatment (Wang, 2000). To assess this, the three enzymes—H. pylori glutamine synthetase (HpGS), R. centenum arginine dihydrolase (RcArgZ), and BpsA—were freeze-dried with different stabilisers. The results showed that all of the enzymes were able to retain activity when freeze-dried (FIGS. 23A-23C).

Mannitol at a concentration of 40 mg/mL was chosen as the best stabiliser to use for collectively freeze-drying the three enzymes. This decision was made in consideration of BpsA activity and stability. The final freeze-drying condition was selected as 50 mM Tris-Cl pH 8.0, 1 mM MgCl₂, and 40 mg/mL mannitol. By mass, 10% of the freeze-dried material was the enzyme and 90% was the lyoprotectants. Given these favourable results, it was concluded that freeze-drying methods are readily applied to the three-enzyme biosensor system.

Activity Trial with Wine Grape Samples

The ability of RcArgZ to detect arginine in wine grape samples (Pinot Noir and Pinot Gris) was verified by coupling with GS and BpsA. Optimised reaction conditions for the two-enzyme GS/BpsA test (see Example 3) were also used in the three-enzyme assay, with untreated wine grape samples.

For these experiments, RcArgZ was coupled with HpGS and S/BpsA for detecting arginine, ammonium and glutamine. The HpGS-BpsA test was used for detecting ammonium and glutamine. The wine grape samples used were Pinot Noir (PN) and Pinot Gris (PG). The RcArgZ-HpGs-BpsA reaction consisted of 3 μM RcArgZ, 6 μM HpGS, 9 μM BpsA, 20 mM MgCl₂, 10 mM KCl, 10 mM glutamate, and 100 mM unbuffered Tris base, pH 11.4. The HpGS-BpsA reaction consisted of 6 μM HpGS, 9 μM BpsA, 20 mM MgCl₂, 10 mM KCl, 10 mM glutamate, and 100 mM Tris pH 11.4.

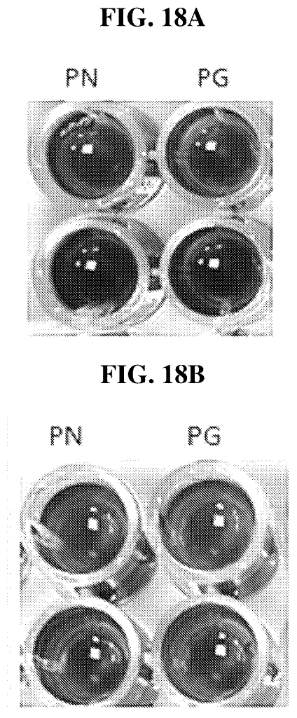

These results demonstrated that RcArgZ was active in detecting arginine in the three-enzyme system. Moreover, the intensity of colour development was significantly greater when RcArgZ was included with HpGS and BpsA (see FIGS. 18A and 18B).

FIGS. 18A-18B show RcArgZ activity in grape wine samples. For FIG. 18A, RcArgZ (detecting arginine) was coupled with HpGS-S/BpsA (detecting ammonium and glutamine) in the assay. For FIG. 18B, the HpGS-S/BpsA test is demonstrated. The wine grape samples used were Pinot Noir (PN) and Pinot Gris (PG). The test used the GS-BpsA optimised condition for ammonium sensing.

The differences in absorbance were collected using a plate-reading spectrophotometer. Table 5 shows the absorbance values between the two tests.

TABLE 5
Absorbance levels from two-enzyme and three-enzyme tests

| | |
|---|---|
| GS-BpsA | ArgZ-GS-BpsA |

| PN (A₅₉₀) | PG (A₅₉₀) | PN (A₅₉₀) | PG (A₅₉₀) |
|---|---|---|---|
| 1.24 | 1.11 | 2.22 | 1.54 |
| 1.25 | 1.25 | 2.05 | 1.46 |

The absorbance values of the duplicate tests were averaged before calculating the concentrations of the YAN constituents. The combined yeast assimilable nitrogen contribution from ammonium plus glutamine, estimated using the HpGS-S/BpsA test, was 150 mg/L nitrogen for PN and 160 mg/L nitrogen for PG. When ammonium, glutamine and arginine were quantified using the RcArgZ-HpGS-S/BpsA biosensor, the yeast assimilable nitrogen from these three sources was found to be 310 mg/L nitrogen for PN and 210 mg/L nitrogen for PG. Therefore, this test demonstrated that the arginine in the PN and PG samples contributed 160 mg/L nitrogen and 50 mg/L nitrogen, respectively.

### Comparison to Commercial Kits

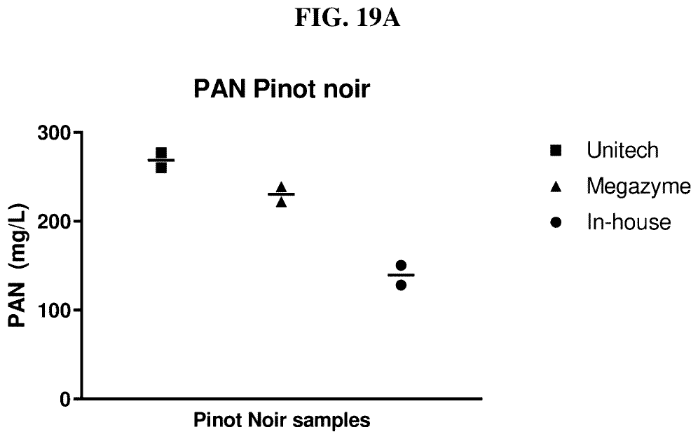

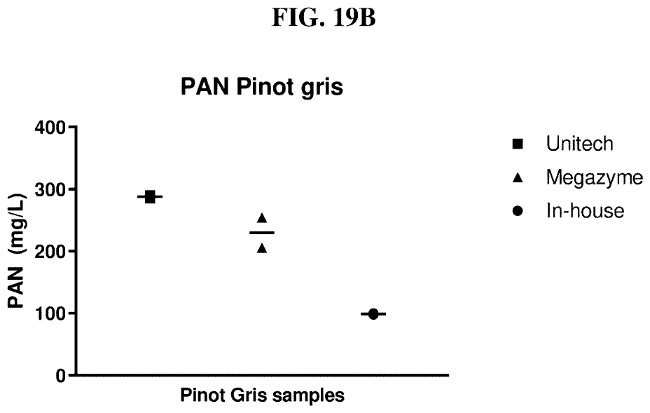

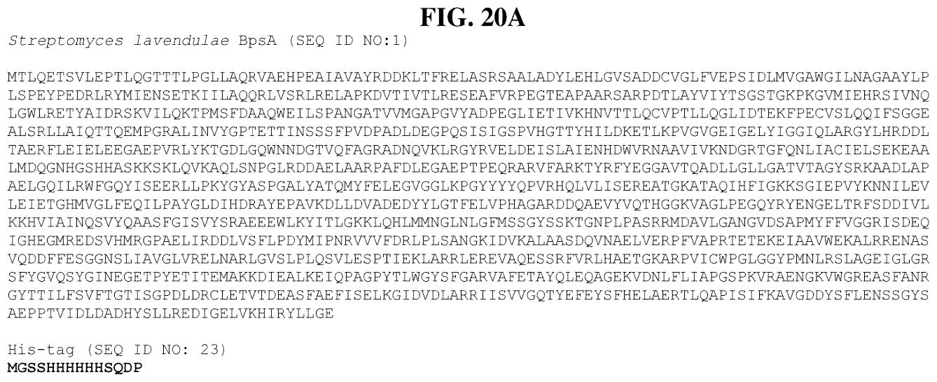

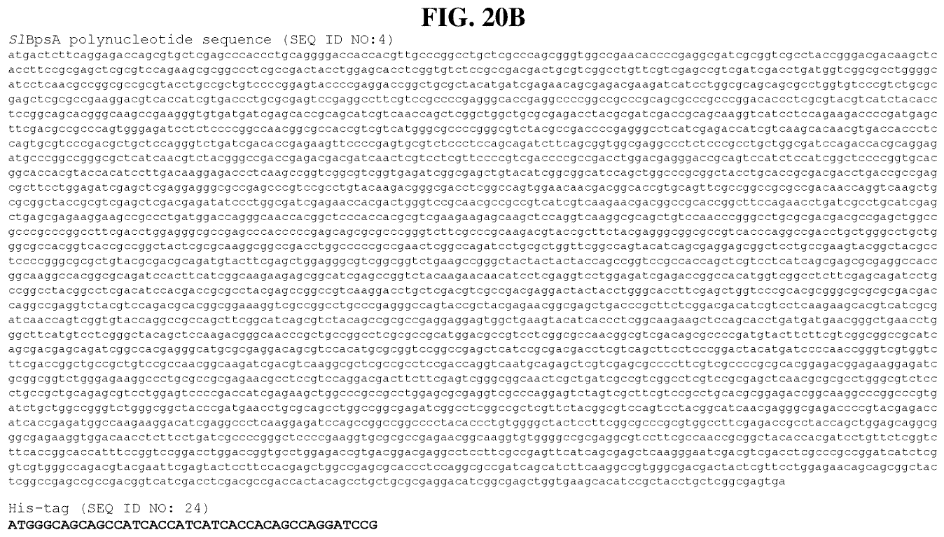

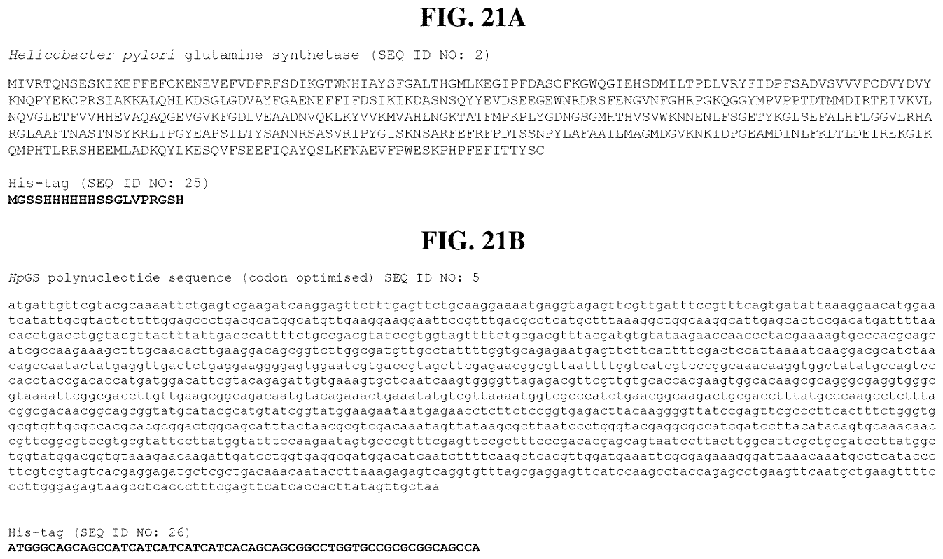

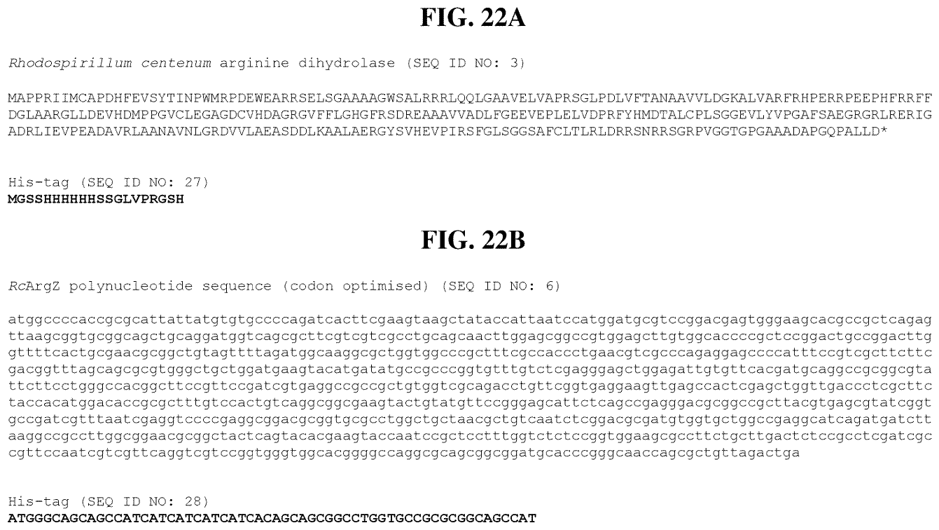

The three-enzyme RcArgZ/HpGS/S/BpsA biosensor system was used to quantify the amino acids arginine, glutamine, and glutamate in Pinot Noir and Pinot Gris grape samples (FIGS. 19A-19B). The same samples were also analysed with two commercial Primary Amino kits (from Unitech and Megazyme). For these experiments, a Pinot Noir grape sample was analysed with the two commercial kits and the multi-enzyme biosensor system, respectively. The experimental procedure is outlined as follows.

Four different reaction mixtures were needed to quantify arginine, glutamine, and glutamate using the disclosed biosensor system. The test using the first reaction mix was used to quantify glutamine. To quantify glutamate, the absorbance at a wavelength of 590 nm (A₅₉₀) of the first reaction mix (quantifying glutamine) was measured and then subtracted from the A₅₉₀ of the second reaction mix (quantifying glutamine and glutamate). Arginine was quantified by subtracting the A₅₉₀ of the third reaction mix (quantifying glutamine and ammonium) from the A₅₉₀ of the fourth reaction mix (quantifying arginine, glutamine, and ammonium).

The first reaction mixture, which was used to quantify glutamine, included 9 μM BpsA, 8 mM ATP, 20 mM MgCl₂, 10 mM KCl, and 0.1 M Tris pH 11.4. The second reaction mixture, which quantified glutamate and glutamine, included 6 μM HpGS, 9 μM BpsA, 8 mM ATP, 20 mM MgCl₂, 10 mM KCl, and 0.1 M Tris pH 11.4. The third reaction mixture quantified glutamine and ammonium. It included 6 μM HpGS, 9 μM BpsA, 8 mM ATP, 20 mM MgCl₂, 10 mM KCl, 10 mM glutamate, and 0.1 M Tris pH 11.4. Lastly, the fourth reaction mixture included 3 μM RcArgZ, 6 μM HpGS, 9 μM BpsA, 8 mM ATP, 20 mM MgCl₂, 10 mM KCl, 10 mM glutamate, and 0.1 M Tris pH 11.4.

In this way, the series of four reactions was used to quantify glutamine, glutamate, ammonium, and arginine. The test was set-up in 96-well microplate format. Each test utilised 35 μL of the respective reaction mixture and 5 μL of the sample. This was incubated for 1 hour at room temperature. After 1 hour, the reaction was stopped and the indigoidine produced was solubilised. This was done by adding 200 μL DMSO, and incubating at room temperature for 20 minutes with shaking at 200 rpm. The absorbance at 590 nm (A₅₉₀) of the solubilised indigoidine was measured using a plate-reading spectrophotometer. The procedure used for the test kits was carried out according to the manufacturers' protocols. The results are shown in FIGS. 19A-19B.

The commercial kits detect all 19 primary amino acids. Therefore, as expected, our three-enzyme sensor reported a lower PAN concentration than the commercial kits. However, it was also apparent that the three amino acids detected by the disclosed biosensor (i.e., arginine, glutamine and glutamate) contributed 40-50% of the total primary amino nitrogen in the Pinot Noir and Pinot Gris juice samples that were tested. These results showed that the disclosed system, which tests for arginine, glutamine and glutamate, is highly effective as a biosensor for the most abundant components of yeast assimilable nitrogen.

In sum, the R. centenum arginine dihydrolase has considerable advantages for use as an arginine biosensor, when coupled with GS and BpsA. Compared with other arginine dihydrolases and arginine deiminases, RcArgZ can be expressed and purified in a highly active form, at high yields. Its characterisation indicated that it is active over a broad pH range with the same pH optimum as GS and BpsA, ensuring that all three enzymes can be used in a single, one pot reaction.

Most importantly, RcArgZ was able to detect and quantify arginine in untreated grape juice samples, without the decolorising or large dilution steps required by commercial kits. When combined with GS and BpsA, the result is an easy-to-use, colorimetric biosensor that reports on the major constituents of YAN. There is substantial utility in employing this biosensor as a semi-quantitative “Yes/No” test as to determine whether YAN levels are acceptable.

### Example 7: Performance Parameters for Biosensor System

A new series of experiments were conducted to determine the performance parameters for the biosensor system. In the first set of studies, experiments was carried out to assess the detection limits for nitrogen using the biosensor system.

At the outset, RcArgZ, HpGS and BpsA enzymes, plus cofactors and reaction buffer, were formulated into biosensor tablets. Fully functional tablets were formulated to range in mass from 25 mg to 120 mg. Details of one particular tablet formulation are provided in Table 6. In Table 6, the masses for the three enzymes refer to the freeze-dried material (see Example 6, above). Each component was mixed using geometric dilution followed by tableting with a TDP 5 Desktop tablet press.

TABLE 6
Exemplification of tablet formulation

| Component | Weight per tablet (mg) |
|---|---|
| ATP | 1.8 |
| KCl | 0.3 |
| MgSO₄ | 1.9 |
| Glutamate | 0.7 |
| Tris base | 4.8 |
| Freeze-dried RcArgZ | 0.6 |
| Freeze-dried HpGS | 2.7 |
| Freeze-dried BpsA | 4.5 |
| Mannitol | 38 |
| Sucrose | 4.7 |

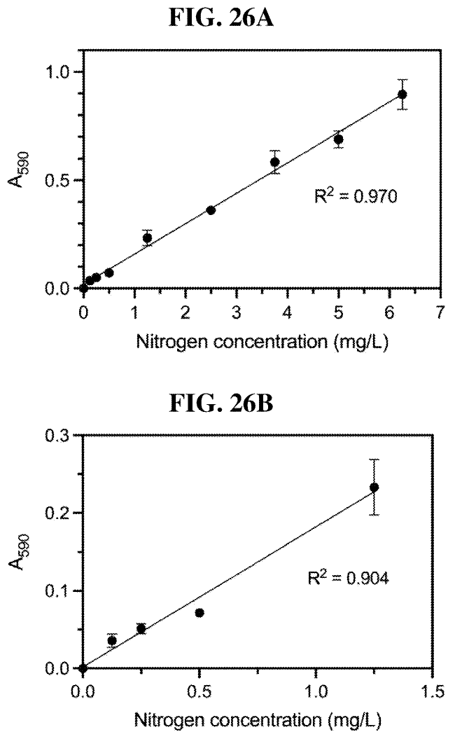

To prepare for the assays, each tablet was dissolved by the addition of water to a final volume of 400 μl. Assays were then carried out. Each assay utilised 39 μl of the reconstituted reaction mixture with 1 μl of a standard solution of ammonium chloride (NH₄Cl), which had been diluted to different concentrations of 19 mg/L, 38 mg/L, 95 mg/L, 191 mg/L, 382 mg/L, 573 mg/L, 764 mg/L, 955 mg/L. The assay mixtures were incubated for 1 hour at 25° C. while shaking at 200 rpm. The indigoidine was resolubilised with 200 μL of DMSO for 20 minutes. Spectrophotometric analysis was then used to determine the limit of detection for nitrogen, this being in the form of NH₄Cl, using the biosensor system. The blue dye, indigoidine, was detected visually and quantified spectrophotometrically by measuring the absorbance at 590 nm (A₅₉₀), as shown in FIGS. 26A-26B.

The amount of indigoidine produced was found to be linear with respect to the concentration of nitrogen (in the form of NH₄Cl) in the assay. The lower limit for detection was found to correspond to a nitrogen concentration of 0.125 mg/L. This corresponds to a molar concentration of 8.9 μM nitrogen. It was noted that the assay had no upper limit for detection, given that a sample may be diluted prior to testing, where desired.

The second set of studies were carried out to assess the effective levels, ratios, and concentrations for the enzymes and other components in the system. At the outset, different concentrations were prepared for HpGS and BpsA for a two-enzyme biosensor system. See Table 7, below.

TABLE 7
Concentrations and ratios of HpGS
and BpsA for two-enzyme biosensor

| Test | HpGS concentration | BpsA concentration | HpGS:BpsA ratio |
|---|---|---|---|
| 1 | 3 μM | 6 μM | 0.50 |
| 2 | 6 μM | 6 μM | 1.00 |
| 3 | 6 μM | 9 μM | 0.67 |
| 4 | 6 μM | 12 μM | 0.50 |

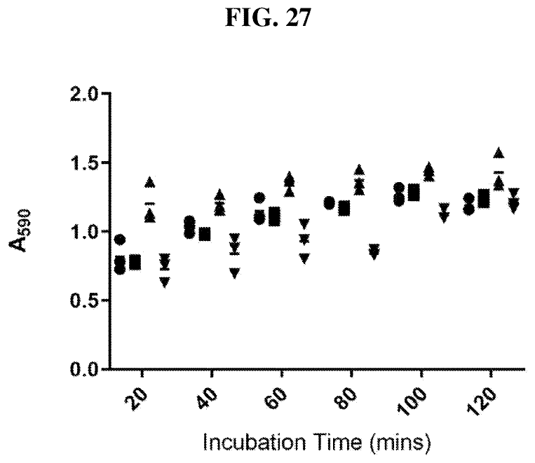

The enzymes at the noted concentrations and ratios (Tests 1-4, Table 7) were used in reaction mixtures containing 100 mM Tris base pH 11.4, 8 mM ATP, 10 mM KCl, 20 mM MgCl₂, and 10 mM glutamate, plus an aliquot (5 μL) of Pinot Gris grape juice as the source of ammonium. Incubations were carried out at 25° C., with samples taken at 20, 40, 60, 80, 100, and 120 minutes. Spectrophotometric assessments were performed for these samples, with the results shown in FIG. 27.

The results indicated that HpGS and BpsA concentrations and ratios can be widely varied, while still providing sensing and reporting on the amount of ammonium in a grape juice sample. In particular, after 120 minutes of incubation, all four tests showed similar final read-outs (i.e., similar values for A₅₉₀). The conditions in Test 3 (6 μM HpGS and 9 μM BpsA) appeared to be most advantageous given that the final A₅₉₀ reading was reached within 20 minutes. However, all tests gave robust read-outs.

To build on these findings, additional testing was carried out to assess more extreme ratios for a three-enzyme biosensor system. Different enzyme concentrations were prepared as noted in Table 8, below.

TABLE 8
Concentrations and ratios for three-enzyme biosensor

| | RcArgZ | | HpGS | | BpsA | | RcArgZ:HpGS:BpsA |
|---|---|---|---|---|---|---|---|
| Test | concentration | | concentration | | concentration | | ratio |
| 5 | 1 | μM | 10 | μM | 100 | μM | 1:10:100 |
| 6 | 100 | μM | 1 | μM | 10 | μM | 100:1:10 |
| 7 | 10 | μM | 100 | μM | 1 | μM | 10:100:1 |

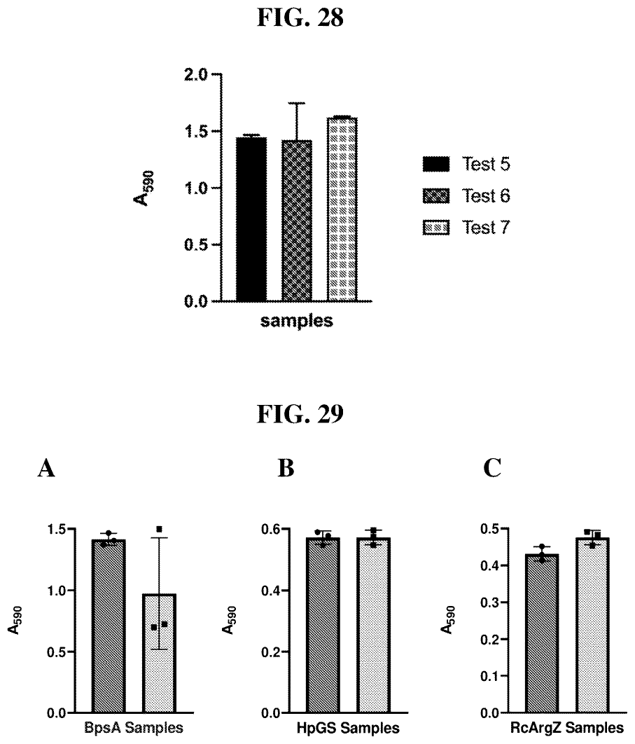

The enzymes at the noted concentrations and ratios (Tests 5-7, Table 8) were used in reaction mixtures containing 100 mM Tris pH 11.4, 20 mM MgCl₂, 10 mM KCl, 15 mM ATP and 1 mM glutamate. Each assay was started by adding 1 mM arginine and incubated for 1 hour at 25° C. DMSO (80% v/v) was added to stop the reaction and resolubilise the indigodine. The results are shown in FIG. 28.

As before, the results indicated that enzyme concentrations and ratios can be widely varied, while still providing sensing and reporting functionality. No significant differences were noted in the amount of indigoidine produced in this assay. This demonstrated that the concentrations and ratios of the enzymes can be altered substantially without detrimental effects.

The third set of studies were carried out to assess the incubation period employed for the biosensor system. Enzymes were either freshly prepared or freeze-dried and then reconstituted. For this testing: (1) BpsA activity was measured in an assay comprising 1 μM enzyme, 15 mM ATP, 20 mM MgCl₂, 10 mM KCl, and 1 mM glutamine. This reaction mixture was incubated for 5 minutes at 37° C. before DMSO was added to 80% (v/v) and the absorbance at 590 nm (A₅₉₀) was measured. (2) HpGS and RcArgZ were assayed under identical conditions, except as follows. For testing HpGS, this enzyme was present at 1 μM, with 5 μM BpsA as a coupled enzyme for colour development. The substrates (NH₄Cl and glutamate) were both present at 1 mM. (3) For testing RcArgZ, this enzyme was present at 1 μM, with the coupled enzymes HpGS and BpsA each present at 5 μM. The substrate arginine and the coupling substrate glutamate were both present at 1 mM. The results are shown in FIGS. 29A-29C.

The results indicated that it was possible to detect an indigoidine signal in as little as 5 minutes using the biosensor system. It was also shown that fresh and freeze dried enzymes performed with similar efficacy. In all cases, absorbance (A₅₉₀) readings greater than 0.4 were obtained with an incubation time of 5 minutes. This A₅₉₀ reading corresponds to an intensity of blue colour that is easily detectable with the naked eye. By way of example, column 1 (“CgGS”) in FIG. 31A shows tests with A₅₉₀ readings of approximately 0.4.

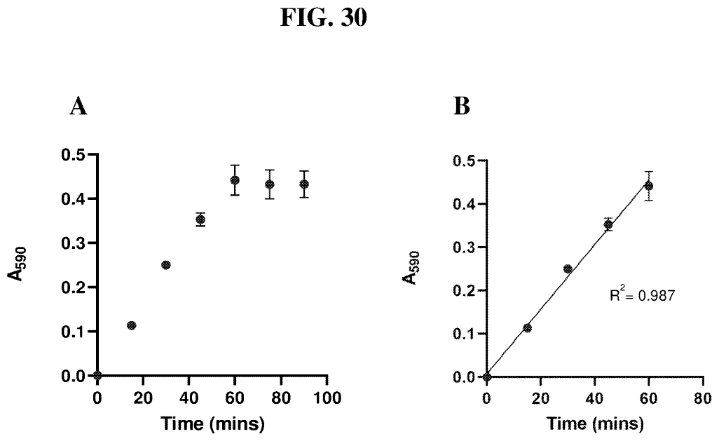

To confirm these findings, a series of tests were carried out using the three-enzyme biosensor, and different time points were assessed. The RcArgZ, HpGS and S/BpsA enzymes, plus cofactors and reaction buffer, were formulated into tablets, as previously. See Example 7, above. To prepare for the assays, each tablet was dissolved by the addition of water to a final volume of 400 μl. Assays were then carried out. Each assay utilised 39 μl of the reconstituted reaction mixture. This reconstituted reaction mixture was mixed with 1 μl of a standard solution of arginine as the substrate for detection. The final concentration of arginine in the assay was equivalent to 150 mg/L nitrogen. Reaction mixtures were incubated at different timepoints (15-90 minutes) at 22° C. before DMSO was added to 80% (v/v). The results are shown in FIGS. 30A-30B.

These results demonstrated that the three-enzyme biosensor system produces indigoidine at a constant rate for 60 minutes. Incubations as short at 5 minutes (FIGS. 29A-29C) or 15 minutes (FIGS. 30A-30B) can be used for testing samples. It is noted that the same incubation time will also be used for standards of known arginine and/or ammonium and/or glutamine concentration. Incubation times greater than 60 minutes lead to no further colour development (FIG. 30A). Therefore, incubation periods that are longer than one hour will still yield an accurate indication of nitrogen levels in a sample.

### Example 8: Performance of Different Enzymes in the Biosensor System

In a first set of studies, different glutamine synthetase enzymes were tested and compared. As described earlier, four GS enzymes were originally assessed for their activity in a biosensor system together with BpsA. See, e.g., Example 1 and Table 1. In this prior assessment, HpGS was identified as the top-ranked enzyme. See, e.g., Example 1. To supplement these findings, additional testing of these four enzymes were performed. Tests were carried out for the four GS enzymes (CgGS, EcGS, HpGS and TcGS) coupled with BpsA in a two-enzyme biosensor. Each reaction mixture included 0.1 M Tris pH 8.5, 20 mM MgCl₂, 1 mM glutamate, and 1 mM NH₄Cl. The GS concentration was 1 μM, and the BpsA concentration was 3 μM. The reactions were incubated for 30 minutes at 25° C. before indigoidine was resolubilised with DMSO. The results are shown in FIGS. 31A-31B. These results demonstrated that both CgGS and HpGS were suitable for utilisation with BpsA in the biosensor system.

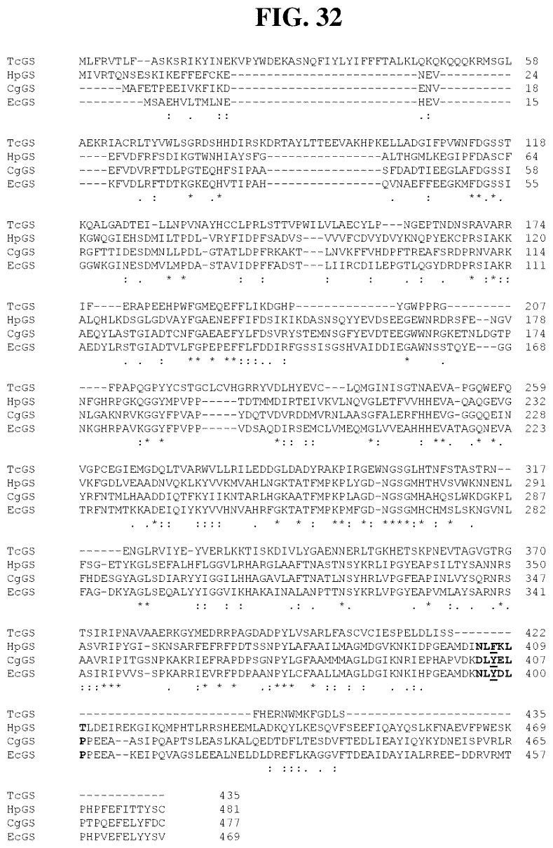

In view of these findings, the amino acid sequences for the four GS candidates, being TcGS, HpGS, CgGS and EcGS were aligned using the Clustal Omega multiple sequence alignment program, version 1.2.4, which is available at https://www.ebi.ac.uk/Tools/msa/clustalo/. The default settings were used and the percentage identity calculations are shown in Table 9. See also FIG. 32.

| | | HpGS | CgGS | EcGS | TcGS |
|---|---|---|---|---|---|
| SEQ ID NO: 2 | HpGS | 100% | 43.1% | 47.9% | 21.2% |
| SEQ ID NO: 13 | CgGS | — | 100% | 51.0% | 20.4% |
| SEQ ID NO: 14 | EcGS | — | — | 100% | 22.8% |
| SEQ ID NO: 15 | TcGS | — | — | — | 100% |

The results for the multiple sequence alignment indicated that HpGS and CgGS exhibit amino acid sequence identity of 43.1% over an aligned length of 477 positions. On the other hand, TcGS was found to be distantly-related to the other three GS enzymes (less than 23% identity shared). It was noted that HpGS, CgGS and EcGS enzymes all contain a conserved sequence motif named the adenylation loop. The sequences of this motif are shown in Table 10, below.

TABLE 10
Conserved sequence motif for GS enzymes

| Enzyme | Motif sequence | SEQ ID NO: |
|---|---|---|
| HpGS | NLFKLT | 16 |
| CgGS | DLYELP | 17 |
| EcGS | NLYDLP | 18 |

The conserved tyrosine, shown in bold and underlined for CgGS and EcGS, is the site of adenylation in these enzymes. HpGS has been deemed unusual because it has a phenylalanine and not a tyrosine at this key position. See, e.g., Joo et al., 2018. This difference at the adenylation site means HpGS is not regulated by adenylation.

Without wishing to be bound by theory, it is hypothesised that this difference is what makes HpGS the top-performing enzyme in our tests. By extension, it should be possible to introduce some or all of the HpGS adenylation motif sequence into other GS enzymes to improve their performance in the biosensor system. For example, the CgGS and EcGS polypeptides as described herein (e.g., SEQ ID NO: 13, 14) could be mutated to substitute the key tyrosine residue (Y) with a phenylalanine residue (F), and thereby reduce or abolish adenylation. See Table 10, above. Accordingly, a useful adenylation site motif may be defined as N/D-L-F-D/E/K-L-P (SEQ ID NO: 19).

In a second set of studies, different arginine dihydrolase enzymes were compared. As described earlier, four ArgZ enzymes were originally assessed for their activity in a biosensor system, and all four enzymes were shown to be active. See, e.g., Example 4 and FIG. 15.

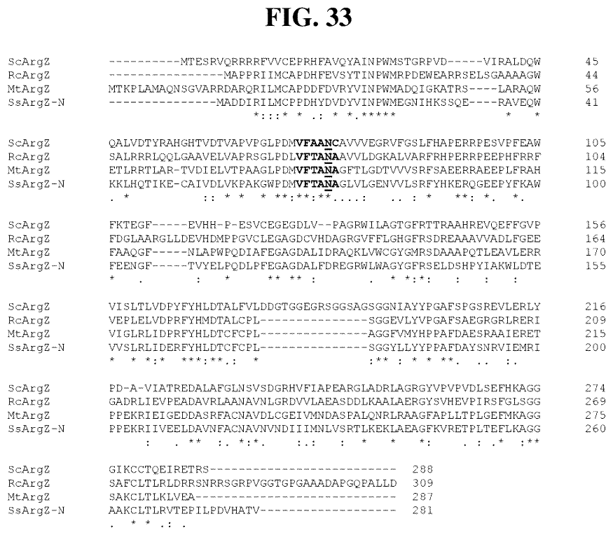

These enzymes (RcArgZ, MtArgZ, ScArgZ, SsArgZ-N) were selected for further assessments. The amino acid sequences were aligned using the Clustal Omega multiple sequence alignment program, version 1.2.4, which is available at https://www.ebi.ac.uk/Tools/msa/clustalo/. The default settings were used and the percentage identity calculations are shown in Table 11. See also FIG. 33.

TABLE 11
Pairwise amino acid sequence identity
calculations for ArgZ enzymes

| | | RcArgZ | MtArgZ | ScArgZ | SsArgZ-N |
|---|---|---|---|---|---|
| SEQ ID NO: 3 | RcArgZ | 100% | 43.5% | 39.3% | 40.4% |
| SEQ ID NO: 9 | MtArgZ | — | 100% | 36.6% | 53.3% |
| SEQ ID NO: 8 | ScArgZ | — | — | 100% | 38.9% |
| SEQ ID NO: 7 | SsArgZ-N | — | — | — | 100% |

The results for the multiple sequence alignment indicated that the RcArgZ enzyme shares similar levels of identity to the MtArgZ, ScArgZ and SsArgZ-N enzymes (e.g., at least 39% amino acid identity). The ScArgZ enzyme appears to be the least closely related to the other three. See Table 11.

It was noted that, for all four enzymes, important active site motifs are conserved. For example, there is a key asparagine residue that defines arginine dihydrolase activity instead of arginine deaminase activity. This residue (underlined and bold in Table 12, below) is present in a conserved motif sequence. Accordingly, a useful arginine dihydrolase motif may be defined as V-F-T/A-A-N-A/C (SEQ ID NO: 20).

TABLE 12
Conserved sequence motif for ArgZ enzymes

| Enzyme | Motif sequence | SEQ ID NO: |
|---|---|---|
| RcArgZ | VFTANA | 23 |
| MtArgZ | VFTANA | 24 |
| SsArgZ-N | VFTANA | 25 |
| ScArgZ | VFAANC | 26 |

Therefore, although RcArgZ has been the predominant enzyme utilised in the present experiments, all four arginine dihydrolase enzymes noted herein (e.g., SEQ ID NO: 3 and 7-9) do have the desired activity, and can be employed in the disclosed biosensor systems.

In sum, the biosensor systems of this disclosure demonstrate excellent adaptability. The biosensor systems are highly effective for detection of a wide range of nitrogen levels, with effectively no upper limit of detection. The systems can be utilised over relatively short (5 minute or less) or long (1 hour or more) testing periods with similar levels of sensitivity and accuracy. Moreover, the biosensor systems can be utilised with various GS and ArgZ enzymes which have considerable divergence in their amino acid sequences. Accordingly, although various optimised conditions have been identified herein, the disclosed biosensor system may be utilised under significantly different conditions while still maintaining suitable levels of efficacy.

### REFERENCES

100% New Zealand. (2019). New Zealand wine: An introduction. Retrieved from https://media.newzealand.com/en/story-ideas/new-zealand-wine/
New Zealand Winegrowers Inc. (2019). Annual Report 2019. Retrieved from https://www.nzwine.com/en/media/statistics/annual-report/
Åström, H., Limén, E., & Strömberg, R. (2004). Acidity of secondary hydroxyls in ATP and adenosine analogues and the question of a 2′,3′-hydrogen bond in ribonucleosides. Journal of the American Chemical Society, 126(45), 14710-14711.
Bell, S.-J. & Henschke, P. A. (2005). Implications of nitrogen nutrition for grapes, fermentation and wine. Australian Journal of Grape and Wine Research, 11, 242-295.
Bender, R. A., Janssen, K. A., Resnick, A. D., Blumenberg, M., Foor, F., & Magasanik, B. (1977). Biochemical parameters of glutamine synthetase from Klebsiella aerogenes. Journal of Bacteriology, 129(2), 1001-1009.
Berges. J., & Mulholland. M. (2008). Enzymes and nitrogen cycling. Nitrogen in the marine environment (pp. 1385-1444).
Boudreau, T. F., 4th, Peck, G. M., O'Keefe, S. F., & Stewart, A. C. (2017). Free amino nitrogen concentration correlates to total yeast assimilable nitrogen concentration in apple juice. Food science & nutrition, 6(1), 119-123.
Brown, A. S., Robins, K. J. & Ackerley, D. F. (2017). A sensitive single-enzyme assay system using the non-ribosomal peptide synthetase BpsA for measurement of L-glutamine in biological samples. Sci Rep, 7, 41745.
Brown. J. R., Masuchi. Y., Robb. F. T., & Doolittle. W. F. (1993). Evolutionary relationships of bacterial and archaeal glutamine synthetase gene. J. Mol. Evol. 38, 566-576.
Crépin, L., Nidelet, T., Sanchez, I., Dequin, S., & Camarasa, C. (2012). Sequential use of nitrogen compounds by Saccharomyces cerevisiae during wine fermentation: a model based on kinetic and regulation characteristics of nitrogen permeases. Applied and Environmental Microbiology, 78(22), 8102-8111.
Crispim, M., Damasceno, F. S., Hernández, A., Barisón, M. J., Pretto Sauter, I., Souza Pavani, R., Santos Moura, A., Pral, E., Cortez, M., Elias, M. C., & Silber, A. M. (2018). The glutamine synthetase of Trypanosoma cruzi is required for its resistance to ammonium accumulation and evasion of the parasitophorous vacuole during host-cell infection. PLoS neglected tropical diseases, 12(1), e0006170.
Eisenberg, D., Gill, H. S., Pfluegl, G. M., & Rotstein, S. H. (2000). Structure-function relationships of glutamine synthetases. Biochim Biophys Acta, 1477(1-2), 122-145.
Fleet, G. H. (2003). Yeast interactions and wine flavour. International Journal of Food Microbiology, 86(1-2), 11-22.
Hannam, K. D., Neilsen, G. H., Forge, T., & Neilsen, D. (2013). The concentration of yeast assimilable nitrogen in Merlot grape juice is increased by N fertilization and reduced irrigation. Canadian Journal of Plant Science, 93(1), 37-45.
Eisenberg, D., Gill, H. S., Pfluegl, G. M., & Rotstein, S. H. (2000). Structure-function relationships of glutamine synthetases. Biochim Biophys Acta, 1477(1-2), 122-145.
Joo, H. K., Park, Y. W., & Jang, Y. Y. et al. (2018). Structural Analysis of Glutamine Synthetase from Helicobacter pylori. Sci Rep 8, 11657
Stadtman, E. R. (2004). Regulation of Glutamine Synthetase Activity. EcoSal Plus, 1(1).
Berges. J., & Mulholland. M. (2008). Enzymes and nitrogen cycling. Nitrogen in the marine environment (pp. 1385-1444).
Beckers, G., Burkovski, A., & Nolden, L. (2001). Glutamate synthase of Corynebacterium glutamicum is not essential for glutamate synthesis and is regulated by the nitrogen status. Microbiology, 147(11), 2961-2970.
Robertson, D., Smith, G., & Alberte, R. (1999). Characterization of a cDNA encoding glutaminesynthetase from the marine diatom Skeletonema costatum (Bacillariophyceae). J. Phycol. 35, 786-797.
Mathis, R., Gamas, P., Meyer, Y., & Cullimore, J. V. (2000). The presence of GSI-like genes inhigher plants: Support for the paralogous evolution of GSI and GSII genes. J. Mol. Evol. 50, 116-122.
Brown. J. R., Masuchi. Y., Robb. F. T., & Doolittle. W. F. (1993). Evolutionary relationships of bacterial and archaeal glutamine synthetase gene. J. Mol. Evol. 38, 566-576.
Robertson, D. L., Smith, G. J., & Alberte, R. S. (2001). Glutamine synthetase in marine algae: New surprises from an old enzyme. J. Phycol. 37, 793-795.
Herrero, A., Muro-Pastor, A. M., & Flores, E. (2001). Nitrogen control in cyanobacteria. J. Bacteriol. 183, 411-425.
Magasanik, B., & Bueno, R. (1985). The role of uridylyltransferase and PII in the regulation of the synthesis of glutamine synthetase in Escherichia coli. Current Topics in Cellular Regulation Modulation by Covalent Modification, 215-220.
Magasanik, B. (1982). Genetic Conrol of nitrogen assimilation in bacteria. Annu Rev Genet. 16: 135-168.
Weiss, V., & Magasanik, B. (1988). Phosphorylation of nitrogen regulator I NR₁ of Escherichia coli. Proc Natl Acad Sci USA. 83:5909-5913.
Keener, J., & Kustu, S. (1988). Protein kinase and phosphoprotein phosphatase activities of nitrogen regulatory proteins NTRB and NTRC of enteric bacteria: roles of the conserved N-terminal domain of NTRC. Proc Natl Acad Sci USA. 85:4976-4980.
Shapiro, B. M., & Stadtman, E. (1970). Glutamine synthetase (Escherichia coli). Methods in Enzymology Metabolism of Amino Acids and Amines Part A, 910-922.
Joo, H. K., Park, Y. W., Jang, Y. Y. et al. (2018). Structural Analysis of Glutamine Synthetase from Helicobacter pylori. Sci Rep 8, 11657.
Deuel, T. F., & Prusiner, S. (1974). Regulation of Glutamine Synthetase from Bacillus subtilis by Divalent Cations, Feedback Inhibitors, and 1-Glutamine. Journal of Biological Chemistry, 249(1), 257-264.
Galkin, A., Kulakova, L., Sarikaya, E., Lim, K., Howard, A., & Herzberg, O. (2004). Structural insight into arginine degradation by arginine deiminase, an antibacterial and parasite drug target. J Biol Chem, 279(14), 14001-14008. doi:10.1074/jbc.M313410200
Kuhn, R., Starr, M. P., Kuhn, D. A., Bauer, H., and Knackmuss, H. J. (1965) Indigoidine and other bacterial pigments related to 3,3′-bipyridyl. Archives of Microbiology 51(1): 71-84.
Lu, X., Galkin, A., Herzberg, O., & Dunaway-Mariano, D. (2004). Arginine deiminase uses an active-site cysteine in nucleophilic catalysis of L-arginine hydrolysis. J Am Chem Soc, 126(17), 5374-5375. doi:10.1021/ja049543p
Mortimer, P. S., Gladys, C., and Hans-Joachim, K. (1966) Appl. Microbiol., 14: 870-872.
Plaitakis, A., Kalef-Ezra, E., Kotzamani, D., Zaganas, I., & Spanaki, C. (2017). The Glutamate Dehydrogenase Pathway and Its Roles in Cell and Tissue Biology in Health and Disease. Biology, 6(1), 11. doi:10.3390/biology6010011
Stasyuk, N. Y., Gayda, G. Z., Fayura, L. R., Boretskyy, Y. R., Gonchar, M. V., & Sibirny, A. A. (2016). Novel arginine deiminase-based method to assay L-arginine in beverages. Food Chem, 201, 320-326. doi:10.1016/j.foodchem.2016.01.093
Takahashi, H., T. Kumagai, et al. (2007) Cloning and characterization of a Streptomyces single module type non-ribosomal peptide synthetase catalyzing a blue pigment synthesis. J. Biol. Chem., 282(12): 9073-9081.
Verma, N., Singh, A. K., & Kaur, P. (2015). Biosensor based on ion selective electrode for detection of L-arginine in fruit juices. Journal of Analytical Chemistry, 70(9), 1111-1115. doi:10.1134/s1061934815090129
Wang, W. (2000). Lyophilization and development of solid protein pharmaceuticals. International Journal of Pharmaceutics, 203(1), 1-60. doi:https://doi.org/10.1016/SO378-5173(00)00423-3
Weber, T. (2010) Making E. coli an erythromycin production plant. Chemistry & Biology, 17(11), 1168-1169.
Zhang, H., Liu, Y., Nie, X., Liu, L., Hua, Q., Zhao, G.-P., & Yang, C. (2018). The cyanobacterial ornithine-ammonia cycle involves an arginine dihydrolase. Nature Chemical Biology, 14(6), 575-581. doi:10.1038/s41589-018-0038-z
Zhuang, N., Zhang, H., Li, L., Wu, X., Yang, C., & Zhang, Y. (2020). Crystal structures and biochemical analyses of the bacterial arginine dihydrolase ArgZ suggests a “bond rotation” catalytic mechanism. J Biol Chem, 295(7), 2113-2124. doi:10.1074/jbc.RA119.011752
Zhybak, M. T., Fayura, L. Y., Boretsky, Y. R., Gonchar, M. V., Sibirny, A. A., Dempsey, E., Korpan, Y. I. (2017). Amperometric L-arginine biosensor based on a novel recombinant arginine deiminase. Microchimica Acta, 184(8), 2679-2686. doi:10.1007/s00604-017-2290-4
SEQ ID NO: 1-28 are set out herein. The database sequence information (including sequences and accession numbers) provided with this description corresponds to the information accessed online as of 9 November, 2021.

Any references cited in this specification are hereby incorporated by reference. All amino acid and nucleotide sequences in the references cited in this specification are hereby incorporated into this disclosure. No admission is made that any reference constitutes prior art. Nor does discussion of any reference constitute an admission that such reference forms part of the common general knowledge in the art, in Australia or in any other country.

Persons of ordinary skill can utilise the disclosures and teachings herein to produce other embodiments and variations without undue experimentation. All such embodiments and variations are considered to be part of this disclosure.

Accordingly, one of ordinary skill in the art will readily appreciate from the disclosure that later modifications, substitutions, and/or variations performing substantially the same function or achieving substantially the same result as embodiments described herein may be utilised according to such related embodiments. Thus, this disclosure is intended to encompass, within its scope, the modifications, substitutions, and variations to processes, manufactures, compositions of matter, compounds, means, methods, and/or steps set out herein.

The description herein may contain subject matter that falls outside of the scope of the claimed invention. This subject matter is included to aid understanding of the invention.

1-125. (canceled)

126. A kit for detecting levels of nitrogen-containing molecules in a sample, the nitrogen-containing molecules being selected from the group consisting of arginine, ammonium, glutamate, glutamine, and combinations thereof, the kit comprising:
(i) an arginine dihydrolase (ArgZ) enzyme, a glutamine synthetase (GS) enzyme, and a blue pigment synthetase A (BpsA) enzyme, or
(ii) a glutamine synthetase (GS) enzyme and a blue pigment synthetase A (BpsA) enzyme.

127. The kit of claim 126, which provides for detecting the levels of the nitrogen-containing molecules
(i) for yeast assimilable nitrogen testing,
(ii) for water testing,
(iii) for soil testing, or
(iv) for biological sample testing.

128. The kit of claim 126, wherein the BpsA enzyme consists of or comprises an amino acid sequence of SEQ ID NO: 1, or an amino acid sequence sharing at least 80%, at least 90%, at least 95%, or at least 99% sequence identity to an amino acid sequence of SEQ ID NO: 1.

129. The kit of claim 128, wherein the BpsA enzyme includes an oxidation domain motif: (i) K-Y/F-G/R/T/Q/A-Y-A/P/G-S-A/P-G-G/A/S-L/I/T/S-Y-P/A/G-V/T-Q (SEQ ID NO: 21); and (ii) G-Y/F/H/A/T/S-Y/H-Y-Y/V-H/Q/D-P-V/F/L/K/A-E/R/H/Q-H/N-R/Q/T/E-L (SEQ ID NO: 22).

130. The kit of claim 126, wherein the GS enzyme consists of or comprises an amino acid sequence of any one of SEQ ID NO: 2 and SEQ ID NO: 13-14, or an amino acid sequence sharing at least 80%, at least 90%, at least 95%, or at least 99% sequence identity to an amino acid sequence of any one of SEQ ID NO: 2 and SEQ ID NO: 13-14.

131. The kit of claim 130, wherein the GS enzyme includes an adenylation site sequence motif N/D-L-F-D/E/K-L-P (SEQ ID NO: 19).

132. The kit of claim 126, wherein the ArgZ enzyme consists of or comprises an amino acid sequence of any one of SEQ ID NO: 3 and SEQ ID NO: 7-9, or an amino acid sequence sharing at least 80%, at least 90%, at least 95%, or at least 99% sequence identity to an amino acid sequence of any one of SEQ ID NO: 3 and SEQ ID NO: 7-9.

133. The kit of claim 132, wherein the ArgZ enzyme includes an arginine dihydrolase motif V-F-T/A-A-N-A/C (SEQ ID NO: 20).

134. The kit of claim 126, which provides a concentration ratio of about 1:1 to about 1:100 for any of the BpsA enzyme, the GS enzyme, or the ArgZ enzyme.

135. The kit of claim 126, wherein the sample comprises grapes, grape must, and/or grape juice.

136. The kit of claim 126, wherein the sample is a fermentation sample, a water sample, a soil sample, or a biological sample.

137. A method of detecting levels of nitrogen-containing molecules in a sample, the nitrogen-containing molecules being selected from the group consisting of arginine, ammonium, glutamate, glutamine, and combinations thereof, the method comprising incubating the sample with:
(i) a glutamine synthetase (GS) enzyme and a blue pigment synthetase A (BpsA) enzyme, the incubating being under conditions whereby the GS enzyme is capable of capable of converting glutamate and ammonium into glutamine, and the BpsA enzyme is capable of converting glutamine to indigoidine pigment, or
(ii) an arginine dihydrolase (ArgZ) enzyme, a glutamine synthetase (GS) enzyme, and a blue pigment synthetase A (BpsA) enzyme,
the incubating being under conditions whereby the ArgZ enzyme is capable of converting arginine to ornithine and ammonium, the GS enzyme is capable of converting glutamate and ammonium into glutamine, and the BpsA enzyme is capable of converting glutamine to indigoidine pigment, and
analyzing the indigoidine pigment produced by quantitative or qualitative means to determine the amount of indigoidine pigment produced, thereby assessing:
for (i), levels of ammonium and glutamine in the sample, or levels of glutamate and glutamine in the sample,
for (ii), levels of arginine, ammonium, and glutamine in the sample, or levels of arginine, glutamate, and glutamine in the sample, and
thereby detecting the levels of the nitrogen-containing molecules in the sample.

138. The method of claim 137, which is utilised to detect the levels of the nitrogen-containing molecules (i) for yeast assimilable nitrogen testing, (ii) for water testing, (iii) for soil testing, or (iv) for biological sample testing.

139. The method of claim 137, wherein the BpsA enzyme consists of or comprises an amino acid sequence of SEQ ID NO: 1, or an amino acid sequence sharing at least 80%, at least 90%, at least 95%, or at least 99% sequence identity to an amino acid sequence of SEQ ID NO: 1.

140. The method of claim 139, wherein the BpsA enzyme includes an oxidation domain motif: (i) K-Y/F-G/R/T/Q/A-Y-A/P/G-S-A/P-G-G/A/S-L/I/T/S-Y-P/A/G-V/T-Q (SEQ ID NO: 21); and (ii) G-Y/F/H/A/T/S-Y/H-Y-Y/V-H/Q/D-P-V/F/L/K/A-E/R/H/Q-H/N-R/Q/T/E-L (SEQ ID NO: 22).

141. The method of claim 137, wherein the GS enzyme consists of or comprises an amino acid sequence of any one of SEQ ID NO: 2 and SEQ ID NO: 13-14, or an amino acid sequence sharing at least 80%, at least 90%, at least 95%, or at least 99% sequence identity to an amino acid sequence of any one of SEQ ID NO: 2 and SEQ ID NO: 13-14.

142. The method of claim 141, wherein the GS enzyme of a preceding aspect, which includes an adenylation site sequence motif N/D-L-F-D/E/K-L-P (SEQ ID NO: 19).

143. The method of claim 137, wherein the ArgZ enzyme consists of or comprises an amino acid sequence of any one of SEQ ID NO: 3 and SEQ ID NO: 7-9, or an amino acid sequence sharing at least 80%, at least 90%, at least 95%, or at least 99% sequence identity to an amino acid sequence of any one of SEQ ID NO: 3 and SEQ ID NO: 7-9.

144. The method of claim 143, wherein the ArgZ enzyme includes an arginine dihydrolase motif V-F-T/A-A-N-A/C (SEQ ID NO: 20).

145. The method of claim 137, which further includes one or more of:
(a) employing a concentration ratio of about 1:1 to about 1:100 for any of the BpsA enzyme, the GS enzyme, or the ArgZ enzyme;
(b) the sample comprises grapes, grape must, and/or grape juice; or
(c) each enzyme is provided simultaneously to the reaction mixture; or each enzyme is provided sequentially to the reaction mixture; or each enzyme is provided in any combination and in any order to the reaction mixture.
